(19) United States
(12) Patent Application Publication
(10) Pub. No.: US 2025/0270336 A1
(43) Pub. Date: Aug. 28, 2025

(54) ANTI-TL1A ANTIBODIES FOR THE TREATMENT OF ULCERATIVE COLITIS AND CROHN'S DISEASE

(71) Applicant: Cephalon LLC, West Chester, PA (US)

(72) Inventors: Alexandra Kropotova, West Chester, PA (US); Stanislav Stoyanov, Malvern, PA (US); Shyam Bhaskerbhai Mehta, Malvern, PA (US); Andrijana Radivojevic, Miami, FL (US)

(73) Assignee: Cephalon LLC, West Chester, PA (US)

(21) Appl. No.: 19/035,531

(22) Filed: Jan. 23, 2025

Related U.S. Application Data
(63) Continuation of application No. PCT/US23/71103, filed on Jul. 27, 2023.
(60) Provisional application No. 63/387,786, filed on Dec. 16, 2022.
(60) Provisional application No. 63/369,591, filed on Jul. 27, 2022.

(51) Int. Cl. C07K 16/28; A61K 39/00

(52) U.S. Cl. CPC C07K 16/2875; A61K 2039/505; C07K 2317/94

(57) ABSTRACT

Methods are provided for the treatment of moderate to severe ulcerative colitis or moderate to severe Crohn's disease with an antibody or antigen-binding fragment thereof that specifically binds to TNF-like ligand 1A (TL1A). Pharmaceutical formulations comprising an antibody or antigen-binding fragment thereof that specifically binds to TL1A are also provided.

### CROSS-REFERENCE TO RELATED APPLICATIONS

This application is a continuation of International Application No. PCT/US2023/071103, filed Jul. 27, 2023, which claims the benefit of priority to U.S. Provisional Application No. 63/369,591, filed Jul. 27, 2022, and U.S. Provisional Application No. 63/387,786, filed Dec. 16, 2022, the entire contents of each, which are incorporated herein by reference for all purposes.

### REFERENCE TO SEQUENCE LISTING SUBMITTED ELECTRONICALLY

This patent application is filed with a sequence listing in electronic format. The Sequence Listing is provided as a file entitled “2025 Jan. 23 01183-0323-OOUS_SequenceListing_St26.xml,” which was created on Jul. 20, 2023, and which is 67,104 bytes in size. The information in the electronic format of the sequence listing is incorporated herein by reference in its entirety.

### BACKGROUND

Inflammatory bowel disease (IBD) is a chronic inflammatory condition of the gastrointestinal (GI) tract that affects 5 million people worldwide. IBD presents as one of the two major forms, ulcerative colitis (UC) or Crohn's disease (CD). In UC, the inflammation is confined to the mucosa of the colon, while in CD, the transmural inflammatory process can affect the entire GI tract from the mouth to the anus, even though it is preferentially located in the terminal ileum and/or right colon (in nearly 70% of forms).

The overall goal of treatment for patients with active UC is to induce and maintain remission and mucosal healing. Treatment of UC consists of anti-inflammatory and immunosuppressant therapies that are chosen to maximize efficacy while avoiding toxicity. While agents used to treat mild to moderate UC are generally well tolerated, as the severity of UC disease increases, so do the potential toxicities of the medications required to manage the disease. Medical therapy for patients with CD is largely influenced by the type, site, and extent of lesions, as well as the presence of local and extraintestinal manifestations and activity of the disease.

In spite of the advances made in recent years in IBD therapeutics, many patients still do not respond, lose response, or are intolerant to currently available treatments for UC and CD. Furthermore, some of the available therapeutic agents for IBD have been associated with significant safety issues. Accordingly, there remains a need to develop and implement highly effective drugs with favorable side effect profiles for patients.

### BRIEF SUMMARY

Provided herein is a method of treating moderate to severe ulcerative colitis or moderate to severe Crohn's disease in a subject in need thereof, comprising administering to the subject an antibody or antigen-binding fragment thereof that specifically binds to TNF-like ligand 1A (TL1A).

Also provided herein is a method of treating ulcerative colitis or Crohn's disease in a subject in need thereof, the method comprising: (a) determining whether or not a subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, and a sphingosine-1-phosphate (S1P) receptor modulator; and (b) if the subject previously had an inadequate response to, loss of response to, or intolerance to the at least one agent, administering to the subject a composition comprising an antibody or antigen-binding fragment thereof that specifically binds to TNF-like ligand 1A (TL1A). In some aspects, the ulcerative colitis is moderate to severe ulcerative colitis and/or the Crohn's disease is moderate to severe Crohn's disease.

Also provided herein is a method of treating ulcerative colitis or Crohn's disease in a subject in need thereof, the method comprising: (a) determining whether or not a subject has moderate to severe ulcerative colitis or moderate to severe Crohn's disease; and (b) if the subject is has moderate to severe ulcerative colitis or moderate to severe Crohn's disease, administering to the subject a composition comprising an antibody or antigen-binding fragment thereof that specifically binds to TNF-like ligand 1A (TL1A).

In some aspects, the antibody or antigen-binding fragment that specifically binds to TL1A comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10. In some aspects, the antibody or antigen-binding fragment of (i) or (ii) comprises an IgG1 constant region.

In some aspects, the subject was treated previously with one or more agents selected from the group consisting of a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA). In some aspects, the subject had an inadequate response to, loss of response to, or intolerance of one or more of the agents. In some aspects, the subject had an inadequate response to, loss of response to, or intolerance of no more than two classes of biologics. In some aspects, the subject is treated concurrently with an oral corticosteroid, an oral 5-ASA, sulfasalazine, azathioprine (AZA), 6 mercaptopurine (6-MP) and/or methotrexate.

In some aspects, the antibody or antigen-binding fragment thereof is administered at a dose of about 300 mg. In some aspects, the antibody or antigen-binding fragment thereof is administered at dose of about 400 mg. In some aspects, the antibody or antigen-binding fragment thereof is administered at dose of about 450 mg. In some aspects, the antibody or antigen-binding fragment thereof is administered at a dose of about 600 mg. In some aspects, the antibody or antigen-binding fragment thereof is administered at a dose of about 750 mg. In some aspects, the antibody or antigen-binding fragment thereof is administered at a dose of about 900 mg. In some aspects, the antibody or antigen-binding fragment thereof is administered at a dose of about 1500 mg. In some aspects, the antibody or antigen-binding fragment thereof is administered at a dose of about 1750 mg. In some aspects, the antibody or antigen-binding fragment thereof is administered at a dose of about 1800 mg. In some aspects, the antibody or antigen-binding fragment thereof is administered at a dose of about 2000 mg. In some aspects, the antibody or antigen-binding fragment thereof is administered at a dose of about 2250 mg. In some aspects, the antibody or antigen-binding fragment thereof is administered at a dose of about 2500 mg.

In some aspects, the antibody or antigen-binding fragment thereof is administered about once every two weeks. In some aspects, the antibody or antigen-binding fragment thereof is administered about once every four weeks. In some aspects, the antibody or antigen-binding fragment thereof is administered about once a month.

In some aspects, the administration comprises administration of a loading dose of the antibody or antigen-binding fragment thereof, followed by administration of one or more induction doses of the antibody or antigen-binding fragment thereof. In some aspects, the administration only comprises administration of one or more induction doses of the antibody or antigen-binding fragment thereof (i.e., the antibody or antigen-binding fragment thereof is administered without a loading dose).

In some aspects, the loading dose comprises about 1500 mg of the antibody or antigen-binding fragment thereof. In some aspects, the loading dose comprises about 1750 mg of the antibody or antigen-binding fragment thereof. In some aspects, the loading dose comprises about 2000 mg of the antibody or antigen-binding fragment thereof. In some aspects, the loading dose comprises about 2250 mg of the antibody or antigen-binding fragment thereof. In some aspects, the loading dose comprises about 2500 mg of the antibody or antigen-binding fragment thereof.

In some aspects, the one or more induction doses (which can follow a loading dose or can be administered without a loading dose) comprises about 300 mg of the antibody or antigen-binding fragment thereof. In some aspects, the one or more induction doses comprises about 400 mg of the antibody or antigen-binding fragment thereof. In some aspects, the one or more induction doses comprises about 450 mg of the antibody or antigen-binding fragment thereof. In some aspects, the one or more induction doses comprises about 600 mg of the antibody or antigen-binding fragment thereof. In some aspects, the one or more induction doses comprises about 750 mg of the antibody or antigen-binding fragment thereof. In some aspects, the one or more induction doses comprises about 900 mg of the antibody or antigen-binding fragment thereof. In some aspects, the one or more induction doses comprises about 1800 mg of the antibody or antigen-binding fragment thereof.

In some aspects, about six induction doses are administered.

In some aspects, the administration occurs over about 16 weeks. In some aspects, the administration of the one or more induction doses occurs over about 14 weeks. In some aspects, the loading dose is administered about 2 weeks before the one or more induction doses are administered.

In some aspects, the administration comprises administering one or more maintenance_doses of the antibody or antigen-binding fragment thereof, and wherein no loading dose and/or induction dose is administered, optionally wherein the maintenance doses are administered about every 2 weeks.

In some aspects, the administration further comprises administering one or more maintenance_doses of the antibody or antigen-binding fragment thereof, optionally wherein the maintenance doses are administered about every 2 weeks.

In some aspects, the administration comprises administering one or more maintenance_doses of the antibody or antigen-binding fragment thereof, and wherein no loading dose and/or induction dose is administered, optionally wherein the maintenance doses are administered about every 4 weeks.

In some aspects, the administration further comprises administering one or more maintenance_doses of the antibody or antigen-binding fragment thereof, optionally wherein the maintenance doses are administered about every 4 weeks.

In some aspects, the one or more maintenance doses comprises about 300 mg or about 900 mg of the antibody or antigen-binding fragment thereof.

In some aspects, the one or more maintenance doses comprises about 300 mg of the antibody or antigen-binding fragment thereof.

In some aspects, the one or more maintenance doses comprises about 400 mg or about 900 mg of the antibody or antigen-binding fragment thereof.

In some aspects, the one or more maintenance doses comprises about 400 mg of the antibody or antigen-binding fragment thereof.

In some aspects, the one or more maintenance doses comprises about 450 mg or about 900 mg of the antibody or antigen-binding fragment thereof.

In some aspects, the one or more maintenance doses comprises about 450 mg of the antibody or antigen-binding fragment thereof.

In some aspects, the one or more maintenance doses comprises about 900 mg of the antibody or antigen-binding fragment thereof.

In some aspects, about twelve maintenance doses are administered.

In some aspects, the administration comprises administering (i) a loading dose of about 2250 mg, (ii) one or more induction doses of about 450 mg, and (iii) one or more maintenance doses of about 300 mg of the antibody or antigen-binding fragment thereof.

In some aspects, the administration comprises administering (i) a loading dose of about 2250 mg, (ii) one or more induction doses of about 900 mg, and (iii) one or more maintenance doses of about 300 mg of the antibody or antigen-binding fragment thereof.

In some aspects, the administration comprises administering (i) a loading dose of about 2250 mg, (ii) one or more induction doses of about 400 mg, and (iii) one or more maintenance doses of about 400 mg of the antibody or antigen-binding fragment thereof.

In some aspects, the administration comprises administering (i) a loading dose of about 2250 mg, (ii) one or more induction doses of about 450 mg, and (iii) one or more maintenance doses of about 450 mg of the antibody or antigen-binding fragment thereof.

In some aspects, the administration comprises administering (i) a loading dose of about 2250 mg, (ii) one or more induction doses of about 900 mg, and (iii) one or more maintenance doses of about 450 mg of the antibody or antigen-binding fragment thereof.

In some aspects, the administration comprises administering (i) a loading dose of about 2250 mg, (ii) one or more induction doses of about 1800 mg, and (iii) one or more maintenance doses of about 300 mg of the antibody or antigen-binding fragment thereof.

In some aspects, the administration comprises administering (i) a loading dose of about 2250 mg, (ii) one or more induction doses of about 450 mg, and (iii) one or more maintenance doses of about 900 mg of the antibody or antigen-binding fragment thereof.

In some aspects, the administration comprises administering (i) a loading dose of about 2250 mg, (ii) one or more induction doses of about 900 mg, and (iii) one or more maintenance doses of about 900 mg of the antibody or antigen-binding fragment thereof.

In some aspects, the administration comprises administering (i) a loading dose of about 2250 mg, (ii) one or more induction doses of about 1800 mg, and (iii) one or more maintenance doses of about 900 mg of the antibody or antigen-binding fragment thereof.

In some aspects, the administration of the one or more maintenance doses occurs over about 24 weeks. In some aspects, the administration of the one or more maintenance doses occurs over about 40 weeks.

In some aspects, the antibody or antigen-binding fragment thereof is formulated at a concentration of about 150 mg/mL. In some aspects, the antibody or antigen-binding fragment thereof is formulated at a concentration of about 200 mg/mL. In some aspects, the antibody or antigen-binding fragment thereof is formulated at a concentration of about 225 mg/mL. In some aspects, the antibody or antigen-binding fragment thereof is formulated at a concentration of about 250 mg/mL.

In some aspects, the antibody or antigen-binding fragment thereof is formulated in a volume of 3 mL. In some aspects, the antibody or antigen-binding fragment thereof is formulated in a volume of 3 mL or less. In some aspects, the antibody or antigen-binding fragment thereof is formulated in a volume of 2 mL or less.

In some aspects, the antibody or antigen-binding fragment thereof is administered to the subject subcutaneously or intravenously. In some aspects, the antibody or antigen-binding fragment thereof is administered subcutaneously.

In some aspects, the antibody or antigen-binding fragment thereof is administered to the subject via a syringe. In some aspects, the syringe is a pre-filled syringe. In some aspects, the pre-filled syringe is in a volume of 3 mL or less. In some aspects, the pre-filled syringe is in a volume of 2 mL or less.

In some aspects, the subject has moderate to severe ulcerative colitis. In some aspects, the administration results in clinical remission. In some aspects, the clinical remission occurs within 14 weeks of the start of the administration. In some aspects, the clinical remission occurs within 38 weeks of the start of the administration.

In some aspects, the subject has moderate to severe Crohn's disease. In some aspects, the administration results in an endoscopic response. In some aspects, the endoscopic response occurs within 14 weeks of the start of the administration. In some aspects, the endoscopic response occurs within 38 weeks of the start of the administration.

Also provided herein is pharmaceutical formulation comprising: (a) about 100 mg/mL to about 250 mg/mL of an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; (b) about 5 mM to about 15 mM Histidine; (c) about 50 mM to about 150 mM Arginine-Hydrochloride (Arg-HCl); (d) about 2.5% (w/v) to about 7.5% (w/v) Sucrose; and (e) about 0.01% (w/v) to about 0.03% (w/v) Polysorbate-80. In some aspects, the antibody or antigen-binding fragment thereof comprises a heavy chain variable region comprising the amino acid sequence of SEQ ID NO: 7 and a light chain variable region comprising the amino acid sequence of SEQ ID NO: 8. In some aspects, the antibody or antigen-binding fragment comprises an IgG1 constant region. In some aspects, the antibody or antigen-binding fragment thereof comprises a heavy chain comprising the amino acid sequence of SEQ ID NO: 9 and a light chain comprising the amino acid sequence of SEQ ID NO: 10.

In some aspects, the pharmaceutical formulation disclosed herein comprises about 100, about 150, about 200, about 225, or about 250 mg/mL of the antibody or antigen-binding fragment thereof. In some aspects, the pharmaceutical formulation disclosed herein comprises about 5 mM, about 10 mM, or about 15 mM Histidine. In some aspects, the pharmaceutical formulation disclosed herein comprises about 50 mM, about 100 mM, or about 150 mM Arginine-Hydrochloride (Arg-HCl). In some aspects, the pharmaceutical formulation disclosed herein comprises about 2.5%, about 5%, or about 7.5% (w/v) Sucrose. In some aspects, the pharmaceutical formulation disclosed herein comprises about 0.01%, about 0.02%, or about 0.03% (w/v) Polysorbate-80.

In some aspects, the pharmaceutical formulation disclosed herein comprises about 250 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation disclosed herein comprises about 200 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation disclosed herein comprises about 150 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In some aspects, the pharmaceutical formulation is lyophilized. In some aspects, the pharmaceutical formulation is liquid. In some aspects, the pharmaceutical formulation has a pH of 6.0±0.5 after storage at room temperature for 24 hours or at 2-8° C. for 24 hours, 72 hours, or 10 days.

In some aspects, the pharmaceutical formulation has an osmolality of from 200 mOsm/kg to 500 mOsm/kg after storage at room temperature for 24 hours or at 2-8° C. for 24 hours, 72 hours, or 10 days. In some aspects, the pharmaceutical formulation has at least 99% antibody monomer content after storage at 2-8° C. for 24 hours, 72 hours, or 10 days. In some aspects, the pharmaceutical formulation has no significant change in charge heterogeneity profile after storage at 2-8° C. for 24 hours, 72 hours, or 10 days. In some aspects, the pharmaceutical formulation has no significant change in purity after storage at room temperature for 24 hours or at 2-8° C. for 24 hours, 72 hours, or 10 days. In some aspects, the pharmaceutical formulation has at least 90% purity after storage at room temperature for 24 hours or at 2-8° C. for 24 hours, 72 hours, or 10 days. In some aspects, the pharmaceutical formulation has no significant change in particle concentration after storage at room temperature for 24 hours or at 2-8° C. for 24 hours, 72 hours, or 10 days. In some aspects, the pharmaceutical formulation has no significant difference in visual appearance after storage at 2-8° C. for up to 36 months. In some aspects, the pharmaceutical formulation disclosed herein has no significant difference in protein concentration, osmolality or viscosity after storage at 2-8° C., 25° C. or 40° C. for up to 36 months. In some aspects, the pharmaceutical formulation disclosed herein has ≥ (more than or equal to) 95% monomer content, ≤ (less than or equal to) 5.0% dimer content, or no significant difference in low molecular weight species content after storage at 2-8° C. for up to 36 months. In some aspects, the pharmaceutical formulation disclosed herein has ≥ (more than or equal to) 90% purity after storage at 2-8° C. for up to 36 months. In some aspects, the pharmaceutical formulation disclosed herein has from 50% to 90% main species content, from 10% to 40% acidic species content, and/or from 0% to 20% basic species content after storage at 2-8° C. for up to 36 months. In some aspects, the pharmaceutical formulation disclosed herein has no significant difference in sub-visible particle content after storage at 2-8° C., 25° C., or 40° C. for up to 36 months. In some aspects, the pharmaceutical formulation disclosed herein has no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulation disclosed herein has from 70% to 135% relative potency measured by enzyme-linked immunosorbent assay (ELISA) after storage at 2-8° C. for up to 36 months. In some aspects, the pharmaceutical formulation disclosed herein has no significant difference in thermal stability after storage at 2-8° C., 25° C., or 40° C. for up to 6 months. In some aspects, the pharmaceutical formulation disclosed herein has no significant difference in thermal stability after storage at 2-8° C. for up to 36 months. In some aspects, the pharmaceutical formulation disclosed herein has no significant difference in secondary and/or tertiary protein structure after storage at 2-8° C., 25° C., or 40° C. for up to 3 months. In some aspects, the pharmaceutical formulation disclosed herein has no significant difference in secondary protein structure after storage at 2-8° C. for up to 36 months. In some aspects, the pharmaceutical formulation disclosed herein has no significant difference in concentration of Polysorbate-80 after storage at 2-8° C. for up to 24 months.

Also provided herein is a container comprising the pharmaceutical formulation disclosed herein. In some aspects, the container is a glass vial. In some aspects, the container is a glass vial having a fill volume of 3 mL. In some aspects, the container is a syringe, e.g., pre-filled syringe. In some aspects, the pre-filled syringe is in a volume of 2 mL or less.

In some aspects, the antibody or antigen-binding fragment thereof is present in the pharmaceutical formulation or container disclosed herein.

In some aspects, provided herein is a method of treating a disease in a subject in need thereof, the method comprising administering to the subject any of the pharmaceutical formulations provided herein, including a pharmaceutical formulation in any of the containers provided herein. In some aspects, the disease is a gastrointestinal disease.

In some aspects, the gastrointestinal disease is inflammatory bowel disease, Crohn's disease, or ulcerative colitis. In some aspects, the gastrointestinal disease is Crohn's disease or ulcerative colitis.

In some aspects, the pharmaceutical formulation is administered intraveneously. In some aspects, the pharmaceutical formulation is administered subcutaneously.

Also provided herein is a composition for use in accordance with the methods disclosed herein.

Also provided herein is pharmaceutical formulation, comprising: (a) 150 mg/mL of an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) 10 mM Histidine; (c) 100 mM Arginine-Hydrochloride (Arg-HCl); (d) 5% (w/v) Sucrose; and (e) 0.02% (w/v) Polysorbate-80. In some aspects, the antibody or antigen-binding fragment of (i) or (ii) comprises an IgG1 constant region.

In some aspects, the pharmaceutical formulation is lyophilized. In some aspects, the pharmaceutical formulation is liquid. In some aspects, the pharmaceutical formulation has a pH of 6.0±0.5 after storage at room temperature for 24 hours or at 2-8° C. for 24 hours, 72 hours, or 10 days.

In some aspects, the pharmaceutical formulation has an osmolality of from 200 mOsm/kg to 500 mOsm/kg after storage at room temperature for 24 hours or at 2-8° C. for 24 hours, 72 hours, or 10 days. In some aspects, the pharmaceutical formulation has at least 99% antibody monomer content after storage at 2-8° C. for 24 hours, 72 hours, or 10 days. In some aspects, the pharmaceutical formulation has no significant change in charge heterogeneity profile after storage at 2-8° C. for 24 hours, 72 hours, or 10 days. In some aspects, the pharmaceutical formulation has no significant change in purity after storage at room temperature for 24 hours or at 2-8° C. for 24 hours, 72 hours, or 10 days. In some aspects, the pharmaceutical formulation has at least 90% purity after storage at room temperature for 24 hours or at 2-8° C. for 24 hours, 72 hours, or 10 days. In some aspects, the pharmaceutical formulation has no significant change in particle concentration after storage at room temperature for 24 hours or at 2-8° C. for 24 hours, 72 hours, or 10 days. In some aspects, the pharmaceutical formulation has no significant difference in visual appearance after storage at 2-8° C. for up to 36 months. In some aspects, the pharmaceutical formulation disclosed herein has no significant difference in protein concentration, osmolality or viscosity after storage at 2-8° C., 25° C. or 40° C. for up to 36 months. In some aspects, the pharmaceutical formulation disclosed herein has ≥ (more than or equal to) 95% monomer content, ≤ (less than or equal to) 5.0% dimer content, or no significant difference in low molecular weight species content after storage at 2-8° C. for up to 36 months. In some aspects, the pharmaceutical formulation disclosed herein has ≥ (more than or equal to) 90% purity after storage at 2-8° C. for up to 36 months. In some aspects, the pharmaceutical formulation disclosed herein has from 50% to 90% main species content, from 10% to 40% acidic species content, and/or from 0% to 20% basic species content after storage at 2-8° C. for up to 36 months. In some aspects, the pharmaceutical formulation disclosed herein has no significant difference in sub-visible particle content after storage at 2-8° C., 25° C., or 40° C. for up to 36 months. In some aspects, the pharmaceutical formulation disclosed herein has no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulation disclosed herein has from 70% to 135% relative potency measured by enzyme-linked immunosorbent assay (ELISA) after storage at 2-8° C. for up to 36 months. In some aspects, the pharmaceutical formulation disclosed herein has no significant difference in thermal stability after storage at 2-8° C., 25° C., or 40° C. for up to 6 months. In some aspects, the pharmaceutical formulation disclosed herein has no significant difference in thermal stability after storage at 2-8° C. for up to 36 months. In some aspects, the pharmaceutical formulation disclosed herein has no significant difference in secondary and/or tertiary protein structure after storage at 2-8° C., 25° C., or 40° C. for up to 3 months. In some aspects, the pharmaceutical formulation disclosed herein has no significant difference in secondary protein structure after storage at 2-8° C. for up to 36 months. In some aspects, the pharmaceutical formulation disclosed herein has no significant difference in concentration of Polysorbate-80 after storage at 2-8° C. for up to 24 months.

Also provided herein is a container comprising the pharmaceutical formulation disclosed herein. In some aspects, the container is a glass vial. In some aspects, the container is a glass vial having a fill volume of 3 mL.

In some aspects, the antibody or antigen-binding fragment thereof is present in the pharmaceutical formulation or container disclosed herein.

Also provided herein is a composition for use in accordance with the methods disclosed herein.

### BRIEF DESCRIPTION OF THE FIGURES

FIG. 1 shows a schematic diagram of the screening, treatment, and follow-up periods of the study described in Example 1. CD=Crohn's disease; FU=follow-up; I/E-inclusion/exclusion; ID=induction dose; LD=loading dose; PBO=placebo; SES-CD-Simple Endoscopic Score for Crohn's Disease; UC=ulcerative colitis.

FIGS. 2A-2C show the percent (%) monomer composition of three TEV-48574 formulations (F1-F3) after storage at 2-8° C. (FIG. 2A), 25° C. (FIG. 2B), and 40° C. (FIG. 2C).

FIGS. 3A-3C show the percent (%) dimer composition of three TEV-48574 formulations (F1-F3) after storage at 2-8° C. (FIG. 3A), 25° C. (FIG. 3B), and 40° C. (FIG. 3C).

FIGS. 4A-4C shows the percent (%) low molecular weight (LWM) protein fragment composition of three TEV-48574 formulations (F1-F3) after storage at 2-8° C. (FIG. 4A), 25° C. (FIG. 4B), and 40° C. (FIG. 4C).

FIGS. 5A-5C show the percent (%) purity of three TEV-48574 formulations (F1-F3) after storage at 2-8° C. (FIG. 5A), 25° C. (FIG. 5B), and 40° C. (FIG. 5C), determined using CGE-Non-reducing conditions (% IgG+125 kDa peak).

FIGS. 6A-6C show the percent (%) purity of three TEV-48574 formulations (F1-F3) after storage at 2-8° C. (FIG. 6A), 25° C. (FIG. 6B), and 40° C. (FIG. 6C), determined using CGE-Reducing conditions (% Heavy chain+light chain).

FIGS. 7A-7C show the charge heterogeneity profile for Formulations 1-3 (F1-F3) after storage at 2-8° C. (FIG. 7A), 25° C. (FIG. 7B), and 40° C. (FIG. 7C), as percent (%) main species (main peak).

FIGS. 8A-8C show the charge heterogeneity profile for Formulations 1-3 (F1-F3) after storage at 2-8° C. (FIG. 8A), 25° C. (FIG. 8B), and 40° C. (FIG. 8C), represented as percent (%) acidic species (acidic peak).

FIGS. 9A-9C show the charge heterogeneity profile for Formulations 1-3 (F1-F3) after storage at 2-8° C. (FIG. 9A), 25° C. (FIG. 9B) and 40° C. (FIG. 9C), represented as percent (%) basic species (basic peak).

FIG. 10A shows the thermal stability of Formulations 1-3 (F1-F3) at time zero (T0) and after storage at 2-8° C., 25° C. and 40° C. for 3 months.

FIG. 10B shows the thermal stability of Formulations 1-3 (F1-F3) at time zero (T0) and after storage at 2-8° C., 25° C. and 40° C. for 6 months.

FIG. 10C shows the thermal stability of Formulations 1-3 (F1-F3) at time zero (T0) and after storage at 2-8° C. for 36 months.

FIG. 11A shows the secondary structure of Formulations 1-3 at time zero (T0) and after 3 months of storage at 2-8° C., 25° C., and 40° C. using far ultraviolet (UV) circular dichroism (CD).

FIG. 11B (shows the secondary structure of Formulations 1-3 at time zero (T0) and after 24 months of storage at 2-8° C. using far UV CD.

FIG. 11C shows the secondary structure of Formulations 1-3 at time zero (T0) and after 36 months of storage at 2-8° C. using far UV CD.

FIG. 11D shows the tertiary structure of Formulations 1-3 at time zero (T0) and after 6 months of storage at 2-8° C., 25° C., and 40° C. using near UV CD.

FIG. 11E shows the tertiary structure of Formulations 1-3 at time zero (T0) and after 24 months of storage at 2-8° C. using near UV CD.

FIG. 11F shows the tertiary structure of Formulations 1-3 at time zero (T0) and after 36 months of storage at 2-8° C. using near UV CD.

FIG. 12 shows a schematic diagram of the screening, treatment, and follow-up periods of the study described in Example 4. CD=Crohn's disease; FU=Follow-up; I/E-Inclusion/Exclusion criteria; MD=Maintenance Dose; SES CD=Simple Endoscopic Score for Crohn's Disease; UC=ulcerative colitis; LTE=long-term extension.

FIG. 13A shows the secondary structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using far UV CD.

FIG. 13B shows the tertiaty structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using near UV CD.

FIG. 14A shows the percent (%) monomer composition of a lyophilized TEV-48574 formulation at 100 mg/mL (F4) and a liquid TEV-48574 formulation at 200 mg/mL (F5) after storage at 2-8° C. for up to 24 months.

FIG. 14B shows the percent (%) dimer composition of a lyophilized TEV-48574 formulation at 100 mg/mL (F4) and a liquid TEV-48574 formulation at 200 mg/mL (F5) after storage at 2-8° C. for up to 24 months.

FIG. 14C shows the percent (%) low molecular weight species of a lyophilized TEV-48574 formulation at 100 mg/mL (F4) and a liquid TEV-48574 formulation at 200 mg/mL (F5) after storage at 2-8° C. for up to 24 months.

FIG. 15A shows the percent (%) purity of two TEV-48574 formulations (F4 and F5) after storage at 2-8° C. for up to 24 months, determined using CGE-Non-reducing conditions (% IgG+125 kDa peak).

FIG. 15B shows the percent (%) purity of two TEV-48574 formulations (F4 and F5) after storage at 2-8° C. for up to 24 months, determined using CGE-Reducing conditions (% Heavy chain (HC)+light chain (LC)).

FIG. 16A shows the charge heterogeneity profile for Formulations 4 and 5 (F4 and F5) after storage at 2-8° C. for up to 24 months as percent (%) main species (main peak).

FIG. 16B shows the charge heterogeneity profile for Formulations 4 and 5 (F4 and F5) after storage at 2-8° C. for up to 24 months, represented as percent (%) acidic species (acidic peak).

FIG. 16C shows the charge heterogeneity profile for Formulations 4 and 5 (F4 and F5) after storage at 2-8° C. for up to 24 months, represented as percent (%) basic species (basic peak).

FIG. 17A shows the secondary structure of Formulation 4 at time zero (T0) and after 3 months (3M), 6 months (6M) and 12 months (12M) of storage at 25° C., and of Formulation 5 at T0, and after 3M and 12M of storage at 25° C. using far UV CD.

FIG. 17B shows the tertiary structure of Formulation 4 at time zero (T0) and after 3 months (3M) and 12 months (12M) of storage at 25° C., and of Formulation 5 at T0, 3M, and after 6 months (6M) and 12M of storage at 25° C. using near near UV CD.

FIG. 18 shows the viscocity at 20° C. for liquid formulations with increasing concentrations of TEV-48574 (0 to 150 mg/mL), with either 100 mM arginine-HCl added as an excipient (1A) or without arginine-HCl (1B) added.

FIG. 19A shows the percent (%) monomer composition of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 19B shows the percent (%) dimer composition of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 19C shows the percent (%) fragments of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks.

FIG. 20A shows the percent (%) purity of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, determined using CGE-Non-reducing conditions (% IgG+125 kDa peak).

FIG. 20B shows the percent (%) purity of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, determined using CGE-Reducing conditions (% Heavy chain (HC)+light chain (LC)).

FIG. 21A shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks as percent (%) main species (main peak).

FIG. 21B shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, represented as percent (%) acidic species (acidic peak).

FIG. 21C shows the charge heterogeneity profile of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl after storage at 40° C. for up to 8 weeks, represented as percent (%) basic species (basic peak).

FIG. 22 shows the thermal stability of two liquid TEV-48574 formulations at 150 mg/mL (1A) and 100 mg/mL (2A) with 100 mM arginine-HCl added, and two liquid TEV-48574 formulations at 150 mg/mL (1B) and 100 mg/mL (2B) without arginine-HCl at time zero (T0).

FIG. 23A shows the total TL1A measured in 3 cynomolgus monkeys treated with a single subcutaneous dose of TEV-48574 at a dose level of 5 mg/kg. Blood sampling was performed at Day 0 (pre-dose), Day 0.25, 1, 2, 3, 4, 5, 7, 9, 11, 14, 21, 28 and 35. Bars represent the mean value +/− standard error of mean (SEM). BLQ (below the limit of quantification) was set to 2.5 pg/mL (0.5*LLOQ (lower limit of quantification)).

FIG. 23B shows the free TL1A measured in 4 cynomolgus monkeys treated with a single subcutaneous dose of TEV-48574 at a dose level of 5 mg/kg. Blood sampling was performed at Day 0 (pre-dose), Day 0.25, 1, 2, 3, 4, 5, 7, 9, 11, 14, 21, 28 and 35. Bars represent the mean value +/− standard error of mean (SEM). BLQ (below the limit of quantification) was set to 2.5 pg/mL (0.5*LLOQ (lower limit of quantification)).

FIG. 24A shows simulated TEV-48574 concentrations for a 2250 mg loading dose and either a Q2W or Q4W maintenance regimen of 900 mg in comparison to RVT-3101 anticipated exposures following administration of 50 mg, 150 mg, or 450 mg on a Q4W regimen.

FIG. 24B shows simulated TEV-48574 concentrations for a 2250 mg loading dose and either a Q2W or Q4W maintenance regimen of 450 mg in comparison to RVT-3101 anticipated exposures following administration of 50 mg, 150 mg, or 450 mg on a Q4W regimen.

FIG. 25 shows a schematic diagram of the screening, treatment, and follow-up periods of the Dose-Ranging Study described in Example 9. CD-Crohn's disease; FU-follow-up; I/E-inclusion/exclusion; ID=induction dose; LD-loading dose; PBO=placebo; SES-CD-Simple Endoscopic Score for Crohn's Disease; UC-ulcerative colitis.

FIG. 26 shows a schematic diagram of the screening, treatment, and follow-up periods of the Long-Term Extension Study described in Example 9. CD=Crohn's disease; FU-Follow-up; I/E-Inclusion/Exclusion criteria; MD=Maintenance Dose; SES CD=Simple Endoscopic Score for Crohn's Disease; UC=ulcerative colitis; LTE=long-term extension.

### DETAILED DESCRIPTION

In order that the present disclosure can be more readily understood, certain terms are first defined. As used in this application, except as otherwise expressly provided herein, each of the following terms shall have the meaning set forth below. Additional definitions are set forth throughout the application.

### Definitions

Various terms relating to aspects of the disclosure are used throughout the specification and claims. Such terms are to be given their ordinary meaning in the art, unless otherwise indicated. Other specifically defined terms are to be construed in a manner consistent with the definition provided herein.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as radioisotopes.

Where not expressly stated, and unless the context indicates otherwise, the term “antibody” includes monospecific, bispecific, or multi-specific antibodies. In some aspects, the antibody is a bispecific antibody. The term “bispecific antibodies” refers to antibodies that bind to two different epitopes.

The term “antibody fragment” refers to a portion of an intact antibody. An “antigen-binding fragment,” “antigen-binding domain,” or “antigen-binding region,” refers to a portion of an intact antibody that binds to an antigen. In the context of a bispecific antibody, an “antigen-binding fragment” binds two antigens. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have the meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In some aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects of the present disclosure, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects of the present disclosure, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91 (3): 969-73 (1994), and Roguska et al., Protein Eng. 9 (10): 895-904 (1996). In some aspects of the present disclosure, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of k_{off}/kₒₙ. Kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects of the present disclosure, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50 (Pt 4): 339-350; McPherson A (1990) Eur J Biochem 189:1-23; Chayen N E (1997) Structure 5:1269-1274; McPherson A (1976) J Biol Chem 251:6300-6303). Crystal structures comprising antigen complexed with an antibody or antigen binding fragment can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49 (Pt 1): 37-60; Bricogne G (1997) Meth Enzymol 276A: 361-423, ed Carter C W; Roversi P et al., (2000) Acta Crystallogr D Biol Crystallogr 56 (Pt 10): 1316-1323). Mutagenesis mapping studies can be accomplished using any method known to one of skill in the art. See, e.g., Champe M et al., (1995) J Biol Chem 270:1388-1394 and Cunningham B C & Wells J A (1989) Science 244:1081-1085 for a description of mutagenesis techniques, including alanine scanning mutagenesis techniques.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects of the present disclosure, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects of the present disclosure, the polypeptides can occur as single chains or associated chains.

As used herein, the term “pharmaceutical formulation” refers to a preparation which is in such form as to permit the biological activity of the active ingredient to be effective, and which contains no additional components which are unacceptably toxic to a subject to which the formulation would be administered. The formulation can be sterile. In some aspects, the formulation is suitable for therapeutic use in a human subject.

The terms “administer,” “administering,” “administration,” and the like, as used herein, refer to methods that can be used to enable delivery of a drug, e.g., an anti-TL1A antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa. and Matucci, A. et al., Respiratory Research, 19 (1): 154 (2018).

As used herein, the terms “combination” or “administered in combination” means that an antibody or antigen binding fragment thereof described herein can be administered with one or more additional therapeutic agents. In some aspects of a combination provided herein, an antibody or antigen binding fragment thereof can be administered with one or more additional therapeutic agents either simultaneously or sequentially. In some aspects, an antibody or antigen binding fragment thereof described herein can be administered with one or more additional therapeutic agent in the same composition or in different compositions.

The terms “subject” and “patient” are used interchangeably and include any animal. In some aspects, the subject is a mammal, including companion (e.g., cat, dog) and farm mammals (e.g., pig, horse, cow), as well as rodents, including mice, rabbits, and rats, guinea pigs, and other rodents. In some aspects, the subject is a non-human primates, such as cynomolgus monkeys. In some aspects, the subject is a human being.

The term “therapeutically effective amount” refers to an amount of a drug, e.g., an anti-TL1A antibody (e.g., TEV-48574) or antigen-binding fragment thereof, effective to treat a disease or disorder in a subject. Terms such as “treating,” “treatment,” “to treat,” “alleviating,” and “to alleviate” refer to utilizing an approach for obtaining beneficial or desired clinical results, including but not limited to an approach that achieves such beneficial or desired clinical results, wherein clinical results can include therapeutic measures that improve, cure, slow down, lessen symptoms of, and/or halt progression of a pathologic condition or disorder. Those in need of treatment can include those already diagnosed with or suspected of having the disorder.

“Specificity” in the context of antibody-antigen interactions is not necessarily an absolute designation but can constitute a relative term signifying the degree of selectivity of an antibody for an antigen-positive cell compared to an antigen-negative cell. Specificity of an antibody for an antigen-positive cell is mediated by the variable regions of the antibody, and usually by the complementarity determining regions (CDRs) of the antibody. A construct can have from about 100 to about 1000-fold specificity for antigen-positive cells compared to antigen-negative cells.

As used herein, the term “recombinant” includes the expression from genes made by genetic engineering or otherwise by laboratory manipulation.

As used herein, the term “TEV 48574” or “TEV-48574” refers to a highly potent, fully human immunoglobulin G (IgG) subclass 1 (IgG1) (lambda) monoclonal antibody (mAb) that binds with high affinity to human, cynomolgus monkey, and rat TL1A. TEV-48574 comprises the light chain of SEQ ID NO: 9 and the heavy chain of SEQ ID NO: 10. TEV-48574 is a blocking antibody that acts by competitively inhibiting the interaction of TL1A to its cognate signaling receptor, DR3. By competitively inhibiting TL1A binding to DR3, the antibody prevents activation of the DR3 signaling pathway. TEV-48574 also inhibits the binding of TL1A to DcR3 although TEV-48574 inhibits the TL1A-DR3 interaction over the TL1A-DcR3 interaction. TEV-48574 has shown anti-inflammatory and anti-fibrotic effects in colitis animal model. TEV-48574 was safe and well tolerated in the Phase 1 study TV48574-SAD-10126. TEV-48574 is disclosed as “320-587” in U.S. Pat. No. 10,138,296, which is herein incorporated by reference in its entirety.

As used herein, the term “TL1A” refers to the Tumor necrosis factor (TNF)-like ligand 1A, also known as TNF superfamily member 15 (TNFSF15) and vascular endothelial growth inhibitor (VEGI), is a member of the TNF superfamily, which is expressed by antigen presenting cells (including dendritic cells, B cells, and macrophages), CD4+ and CD8+ T cells, and endothelial cells. TL1A can be expressed on the cell surface or secreted as a soluble cytokine. The cognate signaling receptor for TL1A, Death Receptor 3 (DR3), is expressed by a variety of cells, including CD4+ and CD8+ T cells, NK cells, NKT cells, and FOXP3+ regulatory T (Treg) cells and type-2 and type-3 innate lymphoid cells (ILC2 and ILC3). TL1A can also bind a decoy receptor (DcR3), which is a competitive inhibitor of DR3. DcR3 also acts as a decoy receptor for Fas-ligand (Fas-L) and lymphotoxin-like inducible protein that competes with glycoprotein D for binding herpesvirus entry mediator on T cells (LIGHT). Accordingly, DcR3 is an important regulator of several signal transduction pathways.

As used herein, “moderate to severe ulcerative colitis,” “moderate to severe ulcerative colitis (UC)” or “moderate to severe UC” refers to the UC with a 3-component modified Mayo score of 5 to 9, inclusive, with an endoscopic subscore of ≥ (more than or equal to) 2 (from central reading).

The 3-component modified Mayo score is based on the following 3 parameters: stool frequency, rectal bleeding, and endoscopic evaluation. Each parameter of the score ranges from 0 (normal or inactive disease) to 3 (severe activity), and the total score ranges from 0 to 9. (Ulcerative Colitis: Clinical Trial Endpoints Guidance for Industry. US Department of Health and Human Services, Food and Drug Administration, Center for Drug Evaluation and Research (CDER). August 2016; Naegeli et al., Am J Gastroenterol; 113: S384 (2018)). Determination of disease severity by the modified Mayo score is recommended by both the Food and Drug Administration (FDA) and the European Medicines Agency (Reinisch et al., Comparison of the EMA and FDA Guidelines on Ulcerative Colitis Drug Development. Clin Gastroenterol Hepatol; 17 (9): 1673-1679.e1 (2019)).

As used herein, “moderate to severe Crohn's disease,” “moderate to severe Crohn's disease (CD)” or “moderate to severe CD” refers to the CD with a Crohn's Disease Activity Index (CDAI) score of ≥ (more than or equal to) 220 and ≤ (less than or equal to) 450. (Yoshida E M. Can J Gastroenterol; 13 (1): 65-73 (1999))

As used herein, the term “inadequate response to, loss of response to, or intolerance of one or more agents” refers to an inadequate response, loss of response, or intolerance to conventional therapy (e.g, small molecules) and/or a biologic agent. The following are provided by way of example.
An inadequate response to, loss of response to, or intolerance to corticosteroid treatment is defined as 1 or more of the following:
   a. Steroid refractory: persistent symptoms of active disease despite treatment with at least one 4-week induction regimen that included a dose of ≥ (more than or equal to) 30 mg prednisone (oral) daily for at least 2 weeks or intravenous (iv) for at least 1 week within the previous 5 years;
   b. Steroid dependent: 2 failed attempts to taper steroids below a dose equivalent to 10 mg prednisone (oral) daily within the previous year; and
   c. Steroid intolerant: history of intolerance to corticosteroids (including, but not limited to, Cushing's syndrome, osteopenia/osteoporosis, hyperglycemia, insomnia, and infection) within the previous 5 years.
An inadequate response to, loss of response to, or intolerance to prior immunosuppressant treatment is defined by 1 or more of the following:
   a. Persistent signs and symptoms of active disease despite a history of at least one 12-week regimen of oral AZA (≥ (more than or equal to) 2 to 2.5 mg/kg/day) or 6-MP (≥ (more than or equal to) 1 to 1.5 mg/kg/day) and/or methotrexate (≥ (more than or equal to) 25 mg/week); and
   b. History of intolerance to AZA, 6-MP, or methotrexate (including, but not limited to, nausea/vomiting, abdominal pain, pancreatitis, liver function testing abnormalities, lymphopenia, thiopurine methyltransferase genetic mutation, and infection).
An inadequate response to, loss of response to, or intolerance to prior biologics or small molecules defined as defined as 1 or more of the following:
   a. Loss of response: Persistent signs and symptoms of active disease despite at least one induction and one maintenance regimen of the locally approved regimen of anti-TNF inhibitors, anti-integrins, anti-IL-12/23 monoclonal antibodies, JAK inhibitors, or S1Preceptor modulators.
   b. Inadequate response (primary non-response): Persistent signs and symptoms of active disease despite at least one induction regimen of the locally approved highest dosing regimen of anti-TNF inhibitors, anti-integrins, anti-IL-12/23 mAbs, JAK inhibitors, or SIP receptor modulators.
   c. Intolerance: Discontinuation of anti-TNF inhibitors, anti-integrins, anti-IL-12/23 monoclonal antibodies, JAK inhibitors, or SIP receptor modulators due to an adverse drug reaction as determined by treating physician. Such adverse drug reactions include, but are not limited to, nausea/vomiting, abdominal pain, pancreatitis, liver function testing abnormalities, lymphopenia, and infections.

As used herein, the term “clinical remission” of ulcerative colitis refers to the clinical remission determined as follows:
Clinical remission is a modified (9-point rectal bleeding, stool frequency, and endoscopy) Mayo score of ≤ (less than or equal to) 2 points, which is defined by: stool frequency subscore of 0 or 1, rectal bleeding subscore of 0, and endoscopic subscore of 0 or 1, where a score of 1 does not include “friability”

As used herein, the term “endoscopic response” in Crohn's disease refers to an endoscopic response determined as follows:
Endoscopic response defined as a reduction from baseline in Simple Endoscopic Score for Crohn's Disease (SES-CD) of at least 50%.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of ±10% of the value or range remain within the intended meaning of the recited value or range. As is understood by one skilled in the art, reference to “about” a value or range herein includes (and describes) instances that are directed to that value or range per se. For example, description referring to “about X” includes description of “X.”

Any compositions or methods provided herein can be combined with one or more of any of the other compositions and methods provided herein.

Units, prefixes, and symbols are denoted in their Système International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

### Methods

The disclosure provides methods for treating moderate to severe ulcerative colitis or moderate to severe Crohn's disease in a subject in need thereof, comprising administering to the subject an antibody or antigen-binding fragment thereof that specifically binds to TNF-like ligand 1A (TL1A). In some aspects, the disclosure provides a method of treating ulcerative colitis or Crohn's disease in a subject in need thereof, comprising: (a) determining whether or not a subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, and a sphingosine-1-phosphate (S1P) receptor modulator; and (b) if the subject previously had an inadequate response to, loss of response to, or intolerance to at least one agent described in part (a), administering to the subject a composition comprising an antibody or antigen-binding fragment thereof that specifically binds to TNF-like ligand 1A (TL1A). In some aspects, the method comprises not administering a composition comprising an antibody or antigen-binding fragment thereof that specifically binds to TL1A if the subject did not previously have an inadequate response to, loss of response to, or intolerance to at least one agent selected from the following group: a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, and a sphingosine-1-phosphate (S1P) receptor modulator.

In some aspects, the disclosure provides a method of treating ulcerative colitis or Crohn's disease in a subject in need thereof, the method comprising: (a) determining whether or not a subject has moderate to severe ulcerative colitis or moderate to severe Crohn's disease; and (b) if the subject has moderate to severe ulcerative colitis or moderate to severe Crohn's disease, administering to the subject a composition comprising an antibody or antigen-binding fragment thereof that specifically binds to TNF-like ligand 1A (TL1A). In some aspects, the method comprises not administering a composition comprising an antibody or antigen-binding fragment thereof that specifically binds to TL1A if the subject does not have have moderate to severe ulcerative colitis or moderate to severe Crohn's disease.

In some aspects, the antibody or antigen-binding fragment thereof inhibits the capability of TL1A to interact with DR3 and, in some aspects, also with DcR3 and, further inhibits the signalling induced by the interaction of TL1A with DR3. In some aspects, the antibody or antigen-binding fragment thereof has enhanced potency relative to antibody 320-179. In some aspects, the antibody or antigen-binding fragment thereof has enhanced affinity for TL1A relative to antibody 320-179.

In some aspects, the antibody or antigen-binding fragment thereof as disclosed herein, comprises a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6. In some aspects, the antibody or antigen-binding fragment thereof is capable of inhibiting the interaction of TL1A with DR3.

In some aspects, the antibody or antigen-binding fragment thereof as disclosed herein, comprises a heavy chain variable region comprising the amino acid sequence of SEQ ID NO: 7. In some aspects, the antibody or antigen-binding fragment thereof comprises a light chain variable region comprising the amino acid sequence of SEQ ID NO: 8. In some aspects, the antibody or antigen-binding fragment thereof comprises a heavy chain variable region comprising the amino acid sequence of SEQ ID NO: 7 and a light chain variable region comprising the amino acid sequence of SEQ ID NO: 8.

In some aspects, the heavy chain variable region is joined to a heavy chain constant region. In some aspects, the heavy chain constant region is an IgG1, IgG2, or IgG4 heavy chain constant region. In some aspects, the heavy chain variable region of SEQ ID NO: 7 is joined to a human IgG1(ΔK) heavy chain constant region (e.g., SEQ ID NO: 14) such that the heavy chain comprises SEQ ID NO: 9. In some aspects, a heavy chain constant region provided herein does not comprise a C-terminal lysine. In some aspects, the light chain variable region of SEQ ID NO: 8 is joined to a lambda human light chain constant region (e.g., SEQ ID NO: 29) such that the light chain comprises SEQ ID NO: 10.

In some aspects, the antibody or antigen-binding fragment thereof as disclosed herein, is a monoclonal antibody or antigen-binding fragment thereof.

In some aspects, the antibody or antigen-binding fragment thereof as disclosed herein, is a recombinant antibody or antigen-binding fragment thereof. In some aspects, the recombinant antibody is a full length antibody. In some aspects, the recombinant antibody is a monoclonal antibody.

In some aspects, the antibody or antigen-binding fragment thereof as disclosed herein, binds to TL1A with enhanced affinity relative to a 320-179 anti-TL1A antibody, which comprises a heavy chain variable region comprising the amino acid sequence of SEQ ID NO: 11 and a light chain variable region comprising the amino acid sequence of SEQ ID NO: 12. In some aspects, the antibody or antigen-binding fragment thereof as disclosed herein, has enhanced potency relative to the 320-179 anti-TL1A antibody. The enhanced potency can be at least about 10-fold, at least about 12-fold, at least about 13-fold, at least about 15-fold, at least about 20-fold, at least about 25-fold, at least about 27-fold, at least about 40-fold greater potency, about 10-fold to about 40-fold, about 12-fold to about 40-fold, about 13-fold to about 40-fold, about 15-fold to about 40-fold, about 20-fold to about 40-fold, about 25-fold to about 40-fold, or about 27-fold to about 40-fold relative to the 320-179 anti-TL1A antibody. Fold-enhancement of potency can be determined according to a TL1A-induced caspase potency assay in TF-1 cells. See, e.g., U.S. Pat. No. 10,138,296. The 320-179 antibody had favourable biophysical properties, was a potent inhibitor of TL1A, and had a low predicted immunogenicity profile. U.S. Pat. No. 10,138,296 and U.S. Publ. No. 2014/0255302 are incorporated by reference herein in their entirety.

In some aspects, the antibody as disclosed herein, is TEV-48574, which is also referred to as the 320-587 antibody in U.S. Pat. No. 10,138,296 (VH is SEQ ID NO: 3 and VL is SEQ ID NO: 4 in that publication).

In some aspects, the antibody or antigen-binding fragment thereof as disclosed herein, comprises a heavy chain variable region comprising the amino acid sequence of SEQ ID NO: 7 and a light chain variable region comprising the amino acid sequence of SEQ ID NO: 8 and binds to TL1A with enhanced affinity relative to anti-TL1A 320-179 antibody, which comprises a heavy chain variable region comprising the amino acid sequence of SEQ ID NO: 11 and a light chain variable region comprising the amino acid sequence of SEQ ID NO: 12. The anti-TL1A 320-179 antibody has been previously described in U.S. Pat. No. 10,138,296 (VH is SEQ ID NO: 1 and VL is SEQ ID NO: 2 in that publication) and in U.S. Publ. No. 2014/0255302 (VH is SEQ ID NO: 186 and VL is SEQ ID NO: 199 in that publication). The 320-179 antibody had favourable biophysical properties, was a potent inhibitor of TL1A, and had a low predicted immunogenicity profile. U.S. Pat. No. 10,138,296 and U.S. Publ. No. 2014/0255302 are incorporated by reference herein in their entirety.

In some aspects, the antibody or antigen-binding fragment thereof as disclosed herein comprises a heavy chain variable region comprising the amino acid sequence of SEQ ID NO: 7 and a light chain variable region comprising the amino acid sequence of SEQ ID NO: 8 and has enhanced potency relative to the 320-179 anti-TL1A antibody. The enhanced potency can be at least about 10-fold, at least about 12-fold, at least about 13-fold, at least about 15-fold, at least about 20-fold, at least about 25-fold, at least about 27-fold, or at least about 40-fold greater potency relative to the 320-179 anti-TL1A antibody. Fold-enhancement of potency can be determined according to a TL1A-induced caspase potency assay in TF-1 human erythroleukemic cells (ATCC: CRL-2003). See, e.g., U.S. Pat. No. 10,138,296.

In some aspects, the antibody or antigen-binding fragment thereof as disclosed herein, is a full length antibody. In some aspects, the antibody as disclosed herein, is a monoclonal antibody.

In some aspects, the antibody or antigen-binding fragment thereof disclosed herein comprises a human IgG1 heavy chain constant region, a human IgG2 heavy chain constant region, or a human IgG4 heavy chain constant region, or any allotypes thereof. The human IgG1 heavy chain constant region can be selected from among human IgG1 (SEQ ID NO: 13), human IgG1 (ΔK) (SEQ ID NO: 14), human IgG1 252Y/254T/256E (SEQ ID NO: 15), human IgG1 252Y/254T/256E (ΔK) (SEQ ID NO: 16), human IgG1 L234A/L235A/G237A (SEQ ID NO: 17), human IgG1 L234A/L235A/G237A (ΔK) (SEQ ID NO: 18), human IgG1 L235A/G237A (SEQ ID NO: 19), and human IgG1 L235A/G237A (ΔK) (SEQ ID NO: 20). The human IgG2 heavy chain constant region can be selected from among human IgG2 with or without ΔK (SEQ ID NO: 21 and SEQ ID NO: 22) and human IgG2 A330S/P331S with or without (ΔK) (SEQ ID NO: 23 and SEQ ID NO: 24). The human IgG4 heavy chain constant region can be selected from among human IgG4 S228P (SEQ ID NO: 25), human IgG4 S228P (ΔK) (SEQ ID NO: 26), human IgG4 228P/252Y/254T/256E (SEQ ID NO: 27), and human IgG4 228P/252Y/254T/256E (ΔK) (SEQ ID NO: 28). It will be understood that an IgG4 heavy chain could be used without the stabilizing substitution S228P (e.g., IgG4 with YTE alone, IgG4 with YTE and ΔK, or IgG4 with ΔK alone).

In some aspects, the antibody or antigen-binding fragment thereof as disclosed herein, comprises a human lambda light chain constant region or an allotype thereof. The human light chain lambda constant region can comprise SEQ ID NO: 29.

In some aspects, the antibody or antigen-binding fragment thereof as disclosed herein, binds to human TL1A, and can bind to one or more of cynomolgus monkey TL1A, mouse TL1A, rat TL1A, guinea pig TL1A, cat TL1A, dog TL1A, pig TL1A, or rabbit TL1A. In some aspects, the antibody or antigen-binding fragment thereof can bind to TL1A of multiple different species, for example, if the epitope is shared. In some aspects, human TL1A comprises the amino acid sequence of SEQ ID NO: 34, SEQ ID NO: 35, or SEQ ID NO: 36. In some aspects, cynomolgus monkey TL1A comprises the amino acid sequence of SEQ ID NO: 37. In some aspects, mouse TL1A comprises the amino acid sequence of SEQ ID NO: 38. In some aspects, rat TL1A comprises the amino acid sequence of SEQ ID NO: 39. In some aspects, guinea pig TL1A comprises the amino acid sequence of SEQ ID NO: 40. In some aspects, cat TL1A comprises the amino acid sequence of SEQ ID NO: 41. In some aspects, pig TL1A comprises the amino acid sequence of SEQ ID NO: 42. In some aspects, rabbit TL1A comprises the amino acid sequence of SEQ ID NO: 43. In some aspects, dog TL1A comprises the amino acid sequence of SEQ ID NO: 44.

The antibody or antigen-binding fragment thereof as disclosed herein, has a binding affinity for an epitope on TL1A that includes an equilibrium dissociation constant (K_{D}), which can be measured according to a kinetic exclusion assay, such as a KINEXA® assay (Sapidyne Instruments Inc., Boise, ID). The K_{D} for TL1A binding determined from a kinetic exclusion assay is less than about 1000 pM. In some aspects, the K_{D} for TL1A binding determined from a kinetic exclusion assay is less than about 500 pM, or less than about 400 pM, or less than about 300 pM, or less than about 200 pM. In some aspects, the K_{D} for TL1A binding determined from a kinetic exclusion assay is less than about 100 pM.

The K_{D} for TL1A binding determined from a kinetic exclusion assay can be from about 10 pM to about 100 pM. The K_{D} for TL1A binding determined from a kinetic exclusion assay can be from about 25 pM to about 75 pM. The K_{D} for TL1A binding determined from a kinetic exclusion assay can be from about 30 pM to about 60 pM. The K_{D} for TL1A binding determined from a kinetic exclusion assay can be from about 30 pM to about 50 pM. The K_{D} for TL1A binding determined from a kinetic exclusion assay can be from about 35 pM to about 50 pM. The K_{D} for TL1A binding determined from a kinetic exclusion assay can be from about 36 pM to about 46 pM. The K_{D} for TL1A binding determined from a kinetic exclusion assay can be from about 38 pM to about 44 pM. The K_{D} for TL1A binding determined from a kinetic exclusion assay can be from about 39 pM to about 43 pM. The K_{D} for TL1A binding determined from a kinetic exclusion assay can be from about 40 pM to about 45 pM. The K_{D} for TL1A binding determined from a kinetic exclusion assay can be from about 35 pM to about 42 pM. The K_{D} for TL1A binding determined from a kinetic exclusion assay can be about 40 pM. The K_{D} for TL1A binding determined from a kinetic exclusion assay can be about 41 pM. The K_{D} for TL1A binding determined from a kinetic exclusion assay can be about 42 pM. The kinetic exclusion assay can use the antibody molecule or TL1A molecule as the constant binding partner, and the other molecule as the titrant.

The antibody or antigen-binding fragment thereof as disclosed herein, is capable of binding to TL1A-positive cells. The antibody or antigen-binding fragment thereof as disclosed herein, can bind to a TL1A-positive cell with an EC₅₀ value of less than about 100 nM, less than about 75 nM, less than about 50 nM, less than about 30 nM, less than about 25 nM, less than about 20 nM, less than about 18 nM, less than about 15 nM, less than about 13 nM, or less than about 10 nM.

The antibody or antigen-binding fragment thereof as disclosed herein, can be monoclonal. In some aspects, the antibody or antigen-binding fragment thereof as disclosed herein, is a full length antibody comprising two heavy chains and two light chains. In some aspects, the antibody or antigen-binding fragment thereof as disclosed herein, comprises a derivative or fragment or portion of an antibody that retains the antigen-binding specificity, and also retains most or all of the affinity, of the full length antibody. For example, derivatives can comprise at least one variable region (either a heavy chain or light chain variable region). Derivatives can comprise at least two variable regions, e.g., at least one heavy chain variable region and at least one light chain variable region. Other examples of suitable antibody derivatives and fragments include, without limitation, antibodies with polyepitopic specificity, bispecific antibodies, multi-specific antibodies, diabodies, single-chain molecules, as well as FAb, F(Ab′)2, Fd, Fabc, and Fv molecules, single chain (Sc) antibodies, single chain Fv antibodies (scFv), individual antibody light chains, individual antibody heavy chains, fusions between antibody chains and other molecules, heavy chain monomers or dimers, light chain monomers or dimers, dimers consisting of one heavy and one light chain, and other multimers. Single chain Fv antibodies can be multi-valent. All antibody isotypes can be used to produce antibody derivatives, fragments, and portions. Antibody derivatives, fragments, and/or portions can be recombinantly produced and expressed by any cell type, prokaryotic or eukaryotic.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Typically, the antigen binding properties of an antibody are less likely to be disturbed by changes to FR sequences than by changes to the CDR sequences. Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some aspects, the antibody or antigen-binding fragment thereof as disclosed herein, is fully human. Fully human antibodies are those where the whole molecule is human or otherwise of human origin, or includes an amino acid sequence identical to a human form of the antibody. Fully human antibodies include those obtained from a human V gene library, for example, where human genes encoding variable regions of antibodies are recombinantly expressed. Fully human antibodies can be expressed in other organisms (e.g., mice and xenomouse technology) or cells from other organisms transformed with genes encoding human antibodies. Fully human antibodies can nevertheless include amino acid residues not encoded by human sequences, e.g., mutations introduced by random or site directed mutations.

In some aspects, the antibody or antigen-binding fragment thereof as disclosed herein, comprises non-immunoglobulin derived protein frameworks. For example, reference can be made to Ku & Schutz, Proc. Natl. Acad. Sci. USA, 92:6552-6 (1995), which describes a four-helix bundle protein cytochrome b562 having two loops randomized to create CDRs, which have been selected for antigen binding.

In some aspects, the antibody or antigen-binding fragment thereof as disclosed herein, comprises post-translational modifications or moieties, which can impact antibody activity or stability. These modifications or moieties include, but are not limited to, methylated, acetylated, glycosylated, sulfated, phosphorylated, carboxylated, and amidated moieties and other moieties that are well known in the art. Moieties include any chemical group or combinations of groups commonly found on immunoglobulin molecules in nature or otherwise added to antibodies by recombinant expression systems, including prokaryotic and eukaryotic expression systems.

Examples of side chain modifications contemplated by the disclosure include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues can be modified by the formation of heterocyclic condensation products with reagents such as 2,3-butanedione, phenylglyoxal and glyoxal. The carboxyl group can be modified by carbodiimide activation via O-acylisourea formation followed by subsequent derivation, for example, to a corresponding amide. Sulfydryl groups can be modified by methods such as carboxymethylation with iodoacetic acid or iodoacetamide; performic acid oxidation to cysteic acid; formation of mixed disulfides with other thiol compounds; reaction with maleimide, maleic anhydride or other substituted maleimide; formation of mercurial derivatives using 4-chloromercuribenzoate, 4-chloromercuriphenylsulfonic acid, phenylmercury chloride, 2-chloromercuri-4-nitrophenol and other mercurials; carbamoylation with cyanate at alkaline pH. Tryptophan residues can be modified by, for example, oxidation with N-bromosuccinimide or alkylation of the indole ring with 2-hydroxy-5-nitrobenzyl bromide or sulfenyl halides. Tyrosine residues on the other hand, can be altered by nitration with tetranitromethane to form a 3-nitrotyrosine derivative. Modification of the imidazole ring of a histidine residue can be accomplished by alkylation with iodoacetic acid derivatives or N-carbethoxylation with diethylpyrocarbonate.

In some aspects, the antibody or antigen-binding fragment thereof as disclosed herein, includes one or more modifications that modulate serum half-life and biodistribution, including without limitation, modifications that modulate the antibody's interaction with the neonatal Fc receptor (FcRn), a receptor with a key role in protecting IgG from catabolism, and maintaining high serum antibody concentration. Serum half-life modulating modifications can occur in the Fc region of IgG1, IgG2, or IgG4, including the triple substitution of M252Y/S254T/T256E (the “YTE” substitutions, with numbering according to the EU numbering system (Edelman, G. M. et al., Proc. Natl. Acad. USA, 63:78-85 (1969)), as described in U.S. Pat. No. 7,083,784. Other substitutions can occur at positions 250 and 428, see e.g., U.S. Pat. No. 7,217,797, as well as at positions 307, 380 and 434, see, e.g., PCT Publ. No. WO 00/042072. Examples of constant domain amino acid substitutions which modulate binding to Fc receptors and subsequent function mediated by these receptors, including FcRn binding and serum half-life, are described in U.S. Publ. Nos. 2009/0142340, 2009/0068175, and 2009/0092599. Antibodies of any class can have the heavy chain C-terminal lysine omitted or removed to reduce heterogeneity (ΔK). The substitution of S228P (EU numbering) in the human IgG4 can stabilize antibody Fab-arm exchange in vivo (Labrin et al., Nature Biotechnology 27 (8): 767-73 (2009)), and this substitution can be present at the same time as the YTE and/or ΔK modifications.

The antibody or antigen-binding fragment thereof as disclosed herein, can be labelled, bound, or conjugated to any chemical or biomolecule moieties. Labelled antibodies can find use in therapeutic, diagnostic, or basic research applications. Such labels/conjugates can be detectable, such as fluorochromes, electrochemiluminescent probes, quantum dots, radiolabels, enzymes, fluorescent proteins, luminescent proteins, and biotin.

The antibody or antigen-binding fragment thereof as disclosed herein, can be derivatized by known protecting/blocking groups to prevent proteolytic cleavage or enhance activity or stability.

Administering the antibody or antigen-binding fragment thereof as disclosed herein, can comprise subcutaneously administering the antibody or antigen-binding fragment thereof. Administering can comprise intravenously administering the antibody or antigen-binding fragment thereof.

In some aspects, the subject is a human subject. In some aspects, the subject is a non-human primate such as a cynomolgus monkey. In some aspects, the subject is a non-human mammal such as a mouse, rat, guinea pig, cat, pig, rabbit, or dog.

In some aspects, the antibody or antigen-binding fragment thereof is administered at a dose of about 300 mg. In some aspects, the antibody or antigen-binding fragment thereof is administered at dose of about 400 mg. In some aspects, the antibody or antigen-binding fragment thereof is administered at dose of about 450 mg. In some aspects, the antibody or antigen-binding fragment thereof is administered at a dose of about 600 mg. In some aspects, the antibody or antigen-binding fragment thereof is administered at a dose of about 750 mg. In some aspects, the antibody or antigen-binding fragment thereof is administered at a dose of about 900 mg. In some aspects, the antibody or antigen-binding fragment thereof is administered at a dose of about 1500 mg. In some aspects, the antibody or antigen-binding fragment thereof is administered at a dose of about 1750 mg. In some aspects, the antibody or antigen-binding fragment thereof is administered at a dose of about 1800 mg. In some aspects, the antibody or antigen-binding fragment thereof is administered at a dose of about 2000 mg. In some aspects, the antibody or antigen-binding fragment thereof is administered at a dose of about 2250 mg. In some aspects, the antibody or antigen-binding fragment thereof is administered at a dose of about 2500 mg. In some aspects, the antibody or antigen-binding fragment thereof as disclosed herein, is administered about once every two weeks. In some aspects, the antibody or antigen-binding fragment thereof as disclosed herein, is administered about once every four weeks. In some aspects, the antibody or antigen-binding fragment thereof is administered about once a month.

In some aspects, the administration comprises administration of a loading dose of the antibody or antigen-binding fragment thereof, followed by administration of one or more induction doses of the antibody or antigen-binding fragment thereof. In some aspects, the administration does not comprise administration of a loading dose of the antibody or antigen-binding fragment thereof.

In some aspects, the loading dose comprises about 1500 mg of the antibody or antigen-binding fragment thereof. In some aspects, the loading dose comprises about 1750 mg of the antibody or antigen-binding fragment thereof. In some aspects, the loading dose comprises about 2000 mg of the antibody or antigen-binding fragment thereof. In some aspects, the loading dose comprises about 2250 mg of the antibody or antigen-binding fragment thereof. In some aspects, the loading dose comprises about 2500 mg of the antibody or antigen-binding fragment thereof. In some aspects, the loading dose is administered about two weeks prior to the administration of the one or more induction doses.

In some aspects, the one or more induction doses comprises about 300 mg of the antibody or antigen-binding fragment thereof. In some aspects, the one or more induction doses comprises about 400 mg of the antibody or antigen-binding fragment thereof. In some aspects, the one or more induction doses comprises about 450 mg of the antibody or antigen-binding fragment thereof. In some aspects, the one or more induction doses comprises about 600 mg of the antibody or antigen-binding fragment thereof. In some aspects, the one or more induction doses comprises about 750 mg of the antibody or antigen-binding fragment thereof. In some aspects, the one or more induction doses comprises about 900 mg of the antibody or antigen-binding fragment thereof. In some aspects, the one or more induction doses comprises about 1800 mg of the antibody or antigen-binding fragment thereof.

In some aspects, about six induction doses are administered.

In some aspects, the administration occurs over about 16 weeks. In some aspects, the administration of the one or more induction doses occurs over about 14 weeks. In some aspects, the loading dose is administered about 2 weeks before the one or more induction doses are administered.

In some aspects, the administration comprises administering one or more maintenance doses of the antibody or antigen-binding fragment thereof, and wherein no loading dose and/or induction dose is administered, optionally wherein the maintenance doses are administered about every 2 weeks.

In some aspects, the administration further comprises administering one or more maintenance doses of the antibody or antigen-binding fragment thereof, optionally wherein the maintenance doses are administered about every 2 weeks.

In some aspects, the administration comprises administering one or more maintenance doses of the antibody or antigen-binding fragment thereof, and wherein no loading dose and/or induction dose is administered, optionally wherein the maintenance doses are administered about every 4 weeks.

In some aspects, the administration further comprises administering one or more maintenance_doses of the antibody or antigen-binding fragment thereof, optionally wherein the maintenance doses are administered about every 4 weeks.

In some aspects, the antibody or antigen-binding fragment thereof as disclosed herein, is administered at a loading dose of about 2250 mg 2 weeks before administering the induction dose of about 1800 mg, which is administered every 2 weeks, e.g., over about 14 weeks.

In some aspects, the antibody or antigen-binding fragment thereof as disclosed herein, is administered at a loading dose of about 2250 mg 2 weeks before administering the induction dose of about 900 mg, which is administered every 2 weeks, e.g., over about 14 weeks.

In some aspects, the antibody or antigen-binding fragment thereof as disclosed herein, is administered at a loading dose of about 2250 mg 2 weeks before administering the induction dose of about 450 mg, which is administered every 2 weeks, e.g., over about 14 weeks.

In some aspects, the antibody or antigen-binding fragment thereof as disclosed herein, is administered at a dose of about 300 mg every 2 weeks.

In some aspects, the antibody or antigen-binding fragment thereof as disclosed herein, is administered at a dose of about 400 mg every 2 weeks.

In some aspects, the antibody or antigen-binding fragment thereof as disclosed herein, is administered at a dose of about 450 mg every 2 weeks.

In some aspects, the antibody or antigen-binding fragment thereof as disclosed herein, is administered at a dose of about 900 mg every 2 weeks.

In some aspects, the administration comprises administering one or more maintenance doses of the antibody or antigen-binding fragment thereof. In some aspects, the administration comprises administering a loading dose of the antibody or antigen-binding fragment thereof, followed by administration of one or more induction doses of the antibody or antigen-binding fragment thereof, followed by the one or more maintenance doses of the antibody or antigen-binding fragment thereof. In some aspects, the administration comprises administering one or more induction doses of the antibody or antigen-binding fragment thereof, followed by the one or more maintenance doses of the antibody or antigen-binding fragment thereof, wherein the administration does not comprise administration of a loading dose of the antibody or antigen-binding fragment thereof. In some aspects, the administration comprises administering one or more maintenance doses of the antibody or antigen-binding fragment thereof, wherein the administration does not comprise administration of a loading dose of the antibody or antigen-binding fragment thereof and does not comprise administration of an induction dose of the antibody or antigen-binding fragment thereof.

In some aspects, the one or more maintenance doses comprises about 300 mg or about 900 mg of the antibody or antigen-binding fragment thereof.

In some aspects, the one or more maintenance doses comprises about 300 mg of the antibody or antigen-binding fragment thereof.

In some aspects, the one or more maintenance doses comprises about 900 mg of the antibody or antigen-binding fragment thereof.

In some aspects, the one or more maintenance doses comprises about 400 mg or about 900 mg of the antibody or antigen-binding fragment thereof. In some aspects, the one or more maintenance doses comprises about 400 mg of the antibody or antigen-binding fragment thereof.

In some aspects, the one or more maintenance doses comprises about 450 mg or about 900 mg of the antibody or antigen-binding fragment thereof. In some aspects, the one or more maintenance doses comprises about 450 mg of the antibody or antigen-binding fragment thereof.

In some aspects, about ten maintenance doses are administered.

In some aspects, about twelve maintenance doses are administered.

In some aspects, the administration comprises administering (i) a loading dose of about 2250 mg, (ii) one or more induction doses of about 450 mg, and (iii) one or more maintenance doses of about 300 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 450 mg administered about every 2 weeks (e.g., over about 14 weeks), and (iii) then at one or more maintenance doses of about 300 mg every 2 weeks (e.g., over about 24 weeks).

In some aspects, the administration comprises administering (i) a loading dose of about 2250 mg, (ii) one or more induction doses of about 900 mg, and (iii) one or more maintenance doses of about 300 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 900 mg administered about every 2 weeks (e.g., over about 14 weeks), and (iii) then at one or more maintenance doses of about 300 mg every 2 weeks (e.g., over about 24 weeks).

In some aspects, the administration comprises administering (i) a loading dose of about 2250 mg, (ii) one or more induction doses of about 1800 mg, and (iii) one or more maintenance doses of about 300 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 1800 mg administered about every 2 weeks (e.g., over about 14 weeks), and (iii) then at one or more maintenance doses of about 300 mg every 2 weeks (e.g., over about 24 weeks).

In some aspects, the administration comprises administering (i) a loading dose of about 2250 mg, (ii) one or more induction doses of about 450 mg, and (iii) one or more maintenance doses of about 900 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 450 mg administered about every 2 weeks (e.g., over about 14 weeks), and (iii) then at one or more maintenance doses of about 900 mg every 2 weeks (e.g., over about 24 weeks).

In some aspects, the administration comprises administering (i) a loading dose of about 2250 mg, (ii) one or more induction doses of about 900 mg, and (iii) one or more maintenance doses of about 900 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 900 mg administered about every 2 weeks (e.g., over about 14 weeks), and (iii) then at one or more maintenance doses of about 900 mg every 2 weeks (e.g., over about 24 weeks).

In some aspects, the administration comprises administering (i) a loading dose of about 2250 mg, (ii) one or more induction doses of about 1800 mg, and (iii) one or more maintenance doses of about 900 mg of the antibody or antigen-binding fragment thereof.

In some aspects, the antibody or antigen-binding fragment thereof as disclosed herein, is administered (i) at a loading dose of about 2250 mg, (ii) about 2 weeks later at one or more induction doses of about 1800 mg administered about every 2 weeks (e.g., over about 14 weeks), and (iii) then at one or more maintenance doses of about 900 mg every 2 weeks (e.g., over about 24 weeks).

In some aspects, the administration comprises administering (i) a loading dose, (ii) one or more induction doses of about 400 mg, and (iii) one or more maintenance doses of about 400 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 400 mg, and (iii) one or more maintenance doses of about 400 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 400 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 400 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 900 mg, and (iii) one or more maintenance doses of about 400 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 900 mg, and (iii) one or more maintenance doses of about 400 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 450 mg, and (iii) one or more maintenance doses of about 450 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 450 mg, and (iii) one or more maintenance doses of about 450 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 450 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 450 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 900 mg, and (iii) one or more maintenance doses of about 450 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 900 mg, and (iii) one or more maintenance doses of about 450 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose, (ii) one or more induction doses of about 900 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of about 2250 mg, (ii) one or more induction doses of about 900 mg, and (iii) one or more maintenance doses of about 900 mg of the antibody or antigen-binding fragment thereof, optionally wherein the induction doses are administered about every two weeks and/or the maintenance doses are administered about every four weeks. In some aspects, about 6 induction doses are administered and/or about ten maintenance doses are administered.

In some aspects, the administration comprises administering (i) a loading dose of and (ii) one or more maintenance doses of about 400 mg of the antibody or antigen-binding fragment thereof, optionally wherein the maintenance doses are administered about every four weeks. In some aspects, about ten maintenance doses are administered. In some aspects, at least ten (e.g., ten to fifty or ten to twenty-four, or ten to twenty, or ten to twelve) maintenance doses are administered.

In some aspects, the administration comprises administering (i) a loading dose of about 2250 mg and (ii) one or more maintenance doses of about 400 mg of the antibody or antigen-binding fragment thereof, optionally wherein the maintenance doses are administered about every four weeks. In some aspects, about ten maintenance doses are administered. In some aspects, at least ten (e.g., ten to fifty or ten to twenty-four, or ten to twenty, or ten to twelve) maintenance doses are administered.

In some aspects, the administration comprises administering (i) a loading dose of and (ii) one or more maintenance doses of about 450 mg of the antibody or antigen-binding fragment thereof, optionally wherein the maintenance doses are administered about every four weeks. In some aspects, about ten maintenance doses are administered. In some aspects, at least ten (e.g., ten to fifty or ten to twenty-four, or ten to twenty, or ten to twelve) maintenance doses are administered.

In some aspects, the administration comprises administering (i) a loading dose of about 2250 mg and (ii) one or more maintenance doses of about 450 mg of the antibody or antigen-binding fragment thereof, optionally wherein the maintenance doses are administered about every four weeks. In some aspects, about ten maintenance doses are administered. In some aspects, at least ten (e.g., ten to fifty or ten to twenty-four, or ten to twenty, or ten to twelve) maintenance doses are administered.

In some aspects, the administration comprises administering (i) a loading dose of and (ii) one or more maintenance doses of about 900 mg of the antibody or antigen-binding fragment thereof, optionally wherein the maintenance doses are administered about every four weeks. In some aspects, about ten maintenance doses are administered. In some aspects, about ten maintenance doses are administered. In some aspects, at least ten (e.g., ten to fifty or ten to twenty-four, or ten to twenty, or ten to twelve) maintenance doses are administered.

In some aspects, the administration comprises administering (i) a loading dose of about 2250 mg and (ii) one or more maintenance doses of about 900 mg of the antibody or antigen-binding fragment thereof, optionally wherein the maintenance doses are administered about every four weeks. In some aspects, about ten maintenance doses are administered. In some aspects, about ten maintenance doses are administered. In some aspects, at least ten (e.g., ten to fifty or ten to twenty-four, or ten to twenty, or ten to twelve) maintenance doses are administered.

In some aspects, the antibody or antigen-binding fragment thereof is formulated at a concentration of about 100 mg/ml or about 150 mg/mL. In some aspects, the antibody or antigen-binding fragment thereof is formulated at a concentration of about 150 mg/mL. In some aspects, the antibody or antigen-binding fragment thereof is formulated at a concentration of about 200 mg/mL. In some aspects, the antibody or antigen-binding fragment thereof is formulated at a concentration of about 225 mg/mL. In some aspects, the antibody or antigen-binding fragment thereof is formulated at a concentration of about 250 mg/mL.

In some aspects, the antibody or antigen-binding fragment thereof is formulated in a volume of 3 mL. In some aspects, the antibody or antigen-binding fragment thereof is formulated in a volume of 3 mL or less. In some aspects, the antibody or antigen-binding fragment thereof is formulated in a volume of 2 mL or less.

In some aspects, the antibody or antigen-binding fragment thereof as disclosed herein, is present in a pharmaceutical formulation or container disclosed herein.

In some aspects, the subject was treated previously with one or more agents selected from the group consisting of a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA). In some aspects, the subject had an inadequate response to, loss of response to, or intolerance of one or more of the agents. In some aspects, the subject had an inadequate response to, loss of response to, or intolerance of no more than two classes of biologics.

In some aspects, the subject is treated concurrently with an oral corticosteroid, an oral 5-ASA, sulfasalazine, azathioprine (AZA), 6 mercaptopurine (6-MP) and/or methotrexate.

In some aspects, the method as disclosed herein, further comprises administering an oral corticosteroid, an oral 5-ASA, sulfasalazine, azathioprine (AZA), 6 mercaptopurine (6-MP) and/or methotrexate.

The disclosure further provides compositions for use in accordance with any method disclosed herein.

### Polynucleotides and Vectors

Polynucleotide sequences that encode the recombinant antibodies or antigen-binding fragments thereof and their subdomains (e.g., FRs and CDRs) are disclosed herein. Polynucleotides include, but are not limited to, RNA, DNA, cDNA, hybrids of RNA and DNA, and single, double, or triple stranded strands of RNA, DNA, or hybrids thereof. Polynucleotides can comprise a nucleic acid sequence encoding the heavy chain variable region and/or the light chain variable region of the antibody as described or exemplified herein. Complements of the polynucleotide sequences are also within the scope of the disclosure.

In some aspects, a polynucleotide can comprise a nucleic acid sequence encoding an antibody heavy chain variable region comprising the amino acid sequence of SEQ ID NO: 7. A polynucleotide encoding the amino acid sequence of SEQ ID NO: 7 can comprise the nucleic acid sequence of SEQ ID NO: 30 or SEQ ID NO: 31.

In some aspects, a polynucleotide can comprise a nucleic acid sequence encoding an antibody light chain variable region comprising the amino acid sequence of SEQ ID NO: 8. A polynucleotide encoding the amino acid sequence of SEQ ID NO: 8 can comprise the nucleic acid sequence of SEQ ID NO: 32 or SEQ ID NO: 33.

In some aspects, a polynucleotide can comprise a first nucleic acid sequence encoding an antibody heavy chain variable region and a second nucleic acid sequence encoding an antibody light chain variable region. A first nucleic acid sequence can encode an antibody heavy chain variable region comprising the amino acid sequence of SEQ ID NO: 7, and a second nucleic acid sequence can encode an antibody light chain variable region comprising the amino acid sequence of SEQ ID NO: 8. A first nucleic acid sequence encoding the amino acid sequence of SEQ ID NO: 7 can comprise the nucleic acid sequence of SEQ ID NO: 30 or SEQ ID NO: 31, and a second nucleic acid sequence encoding the amino acid sequence of SEQ ID NO: 8 can comprise the nucleic acid sequence of SEQ ID NO: 32 or SEQ ID NO: 33.

In some aspects, a polynucleotide can comprise a first nucleic acid sequence encoding an antibody heavy chain variable region and a second nucleic acid sequence encoding a heavy chain constant region. In some aspects, a polynucleotide comprises a first nucleic acid sequence encoding an antibody heavy chain variable region comprising the amino acid sequence of SEQ ID NO: 7 and a second nucleic acid sequence encoding an IgG1(ΔK) heavy chain constant region of SEQ ID NO: 14, for example, a polynucleotide comprising the nucleic acid sequence of SEQ ID NO: 31.

In some aspects, a polynucleotide can comprise a first nucleic acid sequence encoding an antibody light chain variable region and a second nucleic acid sequence encoding a light chain constant region. In some aspects, a polynucleotide comprises a first nucleic acid sequence encoding an antibody light chain variable region comprising the amino acid sequence of SEQ ID NO: 8 and a second nucleic acid sequence encoding a lambda light chain constant region of SEQ ID NO: 29, for example, a polynucleotide comprising the nucleic acid sequence of SEQ ID NO: 33.

Any of the polynucleotides described or exemplified herein can be comprised within a vector. Thus, vectors comprising polynucleotides are provided as part of the disclosure. The vectors can be expression vectors. Recombinant expression vectors containing a sequence encoding a polypeptide of interest are thus provided. The expression vector can contain one or more additional sequences, such as but not limited to regulatory sequences, a selection marker, a purification tag, or a polyadenylation signal. Such regulatory elements can include a transcriptional promoter, enhancers, mRNA ribosomal binding sites, or sequences that control the termination of transcription and translation.

Expression vectors, especially mammalian expression vectors, can include one or more nontranscribed elements, such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a specific host can also be incorporated.

The vectors can be used to transform any of a wide array of host cells well known to those of skill in the art, and host cells capable of expressing antibodies. Vectors include without limitation, plasmids, phagemids, cosmids, bacmids, bacterial artificial chromosomes (BACs), yeast artificial chromosomes (YACs), and baculovirus, as well as other bacterial, eukaryotic, yeast, and viral vectors. Suitable host cells include without limitation CHO cells, NS0 cells, HEK293 cells, or any eukaryotic stable cell line known or produced, and also include bacteria, yeast, and insect cells.

In some aspects, an antibody or antigen-binding fragment thereof provided herein was produced in a human embryonic kidney cell. In some aspects, an antibody or antigen-binding fragment thereof provided herein was produced in a HEK293 cell.

The antibody or antigen-binding fragment thereof can also be produced by hybridoma cells; methods to produce hybridomas being well known and established in the art.

### Pharmaceutical Compositions

The disclosure also provides pharmaceutical compositions comprising the antibodies or antigen-binding fragments thereof provided herein. The pharmaceutical compositions can comprise any of the antibodies or antigen binding fragments thereof described and/or exemplified herein and an acceptable carrier such as a pharmaceutically acceptable carrier. Suitable carriers include any media that does not interfere with the biological activity of the antibody or antigen-binding fragment thereof and is not toxic to a host to which it is administered. The pharmaceutical compositions can be formulated for administration to a subject in any suitable dosage form.

Pharmaceutical compositions suitable for administration to human patients are typically formulated for parenteral administration, e.g., in a liquid carrier, or suitable for reconstitution into liquid solution or suspension for intravenous or subcutaneous administration.

In general, such compositions typically comprise a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans. The term “carrier” refers to a diluent, adjuvant, excipient, or vehicle with which the compound is administered. Such pharmaceutical carriers can be sterile liquids. Water or aqueous solution saline and aqueous dextrose and glycerol solutions can be employed as carriers, particularly for injectable solutions. Liquid compositions for parenteral administration can be formulated for administration by injection or continuous infusion. Routes of administration by injection or infusion include intravenous and subcutaneous.

In some aspects, the pharmaceutical composition comprises: (a) about 100 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80. In some aspects, the pharmaceutical formulation is lyophilized. In some aspects, the pharmaceutical formulation is liquid.

In some aspects, the pharmaceutical formulation comprises: (a) about 150 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80. In some aspects, the pharmaceutical formulation is lyophilized. In some aspects, the pharmaceutical formulation is liquid.

In some aspects, the pharmaceutical formulation comprises: (a) about 100 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80.

In some aspects, the pharmaceutical formulation comprises: (a) about 150 mg/mL of a antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) about 10 mM Histidine; (c) about 100 mM Arginine-Hydrochloride (Arg-HCl); (d) about 5% (w/v) Sucrose; and (e) about 0.02% (w/v) Polysorbate-80.

In some aspects, the pharmaceutical formulations provided herein have a pH of 6.0±0.5 after storage at room temperature for 24 hours or at 2-8° C. for 24 hours, 72 hours, or 10 days. In some aspects, the pharmaceutical formulations provided herein have an osmolality of from 200 mOsm/kg to 500 mOsm/kg after storage at room temperature for 24 hours or at 2-8° C. for 24 hours, 72 hours, or 10 days. In some aspects, the pharmaceutical formulations provided herein have at least 99% antibody monomer content after storage at 2-8° C. for 24 hours, 72 hours, or 10 days. In some aspects, the pharmaceutical formulations provided herein have no significant change in charge heterogeneity profile after storage at 2-8° C. for 24 hours, 72 hours, or 10 days. In some aspects, the pharmaceutical formulations provided herein have no significant change in purity after storage at room temperature for 24 hours or at 2-8° C. for 24 hours, 72 hours, or 10 days. In some aspects, the pharmaceutical formulations provided herein have at least 90% purity after storage at room temperature for 24 hours or at 2-8° C. for 24 hours, 72 hours, or 10 days. In some aspects, the pharmaceutical formulations provided herein have no significant change in particle concentration after storage at room temperature for 24 hours or at 2-8° C. for 24 hours, 72 hours, or 10 days. In some aspects, the pharmaceutical formulations provided herein have no significant difference in visual appearance after storage at 2-8° C. for up to 24 months. In some aspects, the pharmaceutical formulations provided herein have no significant difference in visual appearance after storage at 2-8° C. for up to 36 months. In some aspects, the pharmaceutical formulations provided herein have no significant difference in protein concentration, osmolality or viscosity after storage at 2-8° C., 25° C. or 40° C. for up to 24 months. In some aspects, the pharmaceutical formulations provided herein have no significant difference in protein concentration, osmolality or viscosity after storage at 2-8° C., 25° C. or 40° C. for up to 36 months. In some aspects, the pharmaceutical formulations provided herein have ≥ (more than or equal to) 95% monomer content, ≤ (less than or equal to) 5.0% dimer content, or no significant difference in low molecular weight species content after storage at 2-8° C. for up to 24 months. In some aspects, the pharmaceutical formulations provided herein have ≥ (more than or equal to) 95% monomer content, ≤ (less than or equal to) 5.0% dimer content, or no significant difference in low molecular weight species content after storage at 2-8° C. for up to 36 months. In some aspects, the pharmaceutical formulations provided herein have ≥ (more than or equal to) 90% purity after storage at 2-8° C. for up to 24 months. In some aspects, the pharmaceutical formulations provided herein have ≥ (more than or equal to) 90% purity after storage at 2-8° C. for up to 36 months. In some aspects, the pharmaceutical formulations provided herein have from 50% to 90% main species content, from 10% to 40% acidic species content, and/or from 0% to 20% basic species content after storage at 2-8° C. for up to 24 months. In some aspects, the pharmaceutical formulations provided herein have from 50% to 90% main species content, from 10% to 40% acidic species content, and/or from 0% to 20% basic species content after storage at 2-8° C. for up to 36 months. In some aspects, the pharmaceutical formulations provided herein have no significant difference in sub-visible particle content after storage at 2-8° C., 25° C., or 40° C. for up to 24 months. In some aspects, the pharmaceutical formulations provided herein have no significant difference in sub-visible particle content after storage at 2-8° C., 25° C., or 40° C. for up to 36 months. In some aspects, the pharmaceutical formulations provided herein have no significant difference in oxidation of methionine 81 and/or methionine 254 of TEV-48574, and/or deamidation of asparagine 317 of TEV-48574 after storage at 2-8° C., 25° C., or 40° C. for up to 24 months. In some aspects, the pharmaceutical formulations provided herein have no significant difference in oxidation of methionine 81 and/or methionine 254 of TEV-48574, and/or deamidation of asparagine 317 of TEV-48574 after storage at 2-8° C., 25° C., or 40° C. for up to 36 months. In some aspects, the pharmaceutical formulations provided herein have from 70% to 135% relative potency measured by enzyme-linked immunosorbent assay (ELISA) after storage at 2-8° C. for up to 24 months. In some aspects, the pharmaceutical formulations provided herein have from 70% to 135% relative potency measured by enzyme-linked immunosorbent assay (ELISA) after storage at 2-8° C. for up to 36 months. In some aspects, the pharmaceutical formulations provided herein have no significant difference in thermal stability after storage at 2-8° C., 25° C., or 40° C. for up to 6 months. In some aspects, the pharmaceutical formulations provided herein have no significant difference in thermal stability after storage at 2-8° C. for up to 36 months. In some aspects, the pharmaceutical formulations provided herein have no significant difference in secondary and/or tertiary protein structure after storage at 2-8° C., 25° C., or 40° C. for up to 3 months. In some aspects, the pharmaceutical formulations provided herein have no significant difference in secondary protein structure after storage at 2-8° C. for up to 36 months. In some aspects, the pharmaceutical formulations provided herein have no significant difference in concentration of Polysorbate-80 after storage at 2-8° C. for up to 24 months.

### Kits and Containers

The disclosure also features containers comprising any pharmaceutical formulation described and exemplified herein. In some aspects, the container is a glass vial. In some aspects, the container is a glass vial having a fill volume of 3 mL.

The disclosure also features kits comprising any of the pharmaceutical formulations, or antibodies or antigen-binding fragments thereof described and exemplified herein. The kits can be used to supply pharmaceutical formulations, antibodies, antigen-binding fragments thereof, and other agents for use in diagnostic, basic research, or therapeutic methods, among others. In some aspects, the kits comprise any one or more of the pharmaceutical formulations, antibodies or antigen-binding fragments thereof described or exemplified herein and instructions for using the one or more pharmaceutical formulations, antibodies, or antigen-binding fragments thereof in a method of treating moderate to severe ulcerative colitis or moderate to severe Crohn's disease. In some aspects, the subject was treated previously with one or more agents selected from the group consisting of a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, a sphingosine-1-phosphate (S1P) receptor modulator, and 5-aminosalicylic acid (5-ASA). In some aspects, the subject had an inadequate response to, loss of response to, or intolerance of one or more of the agents. In some aspects, the subject had an inadequate response to, loss of response to, or intolerance of no more than two classes of biologics. In some aspects, the kits comprise any one or more of the pharmaceutical formulations, antibodies, or antigen-binding fragments thereof described or exemplified herein and instructions for using the one or more pharmaceutical formulations, antibodies, or antigen-binding fragments thereof in a method of treating ulcerative colitis or Crohn's disease in a subject in need thereof, the method comprising: (a) determining whether or not a subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, and a sphingosine-1-phosphate (S1P) receptor modulator; and (b) if the subject had an inadequate response, loss of response, or intolerance at least one of the agents, administering to the subject a composition comprising an antibody or antigen-binding fragment thereof that specifically binds to TNF-like ligand 1A (TL1A).

In some aspects, the kits comprise any one or more of the pharmaceutical formulations, recombinant antibodies, or antigen-binding fragments thereof described or exemplified herein and instructions for using the one or more pharmaceutical formulations, antibodies, or antigen-binding fragments thereof in a method of treating ulcerative colitis or Crohn's disease in a subject in need thereof, the method comprising: (a) determining whether or not a subject has moderate to severe ulcerative colitis or moderate to severe Crohn's disease and (b) if the subject has moderate to severe ulcerative colitis or moderate to severe Crohn's disease, administering to the subject a composition comprising an antibody or antigen-binding fragment thereof that specifically binds to TNF-like ligand 1A (TL1A).

### Exemplary Aspects Provided Herein

In one aspect (Aspect 1; A1), provided herein is a method of treating moderate to severe ulcerative colitis or moderate to severe Crohn's disease in a subject in need thereof, comprising administering to the subject an antibody or antigen-binding fragment thereof that specifically binds to TNF-like ligand 1A (TL1A); wherein the antibody or antigen-binding fragment comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10.

In one aspect of A1, i.e., A2, the subject was treated previously with one or more agents selected from the group consisting of a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, a sphingosine-1-phosphate (S1P) receptor modulator, and 5-aminosalicylic acid (5-ASA).

In one aspect of A2, i.e., A3, the subject had an inadequate response to, loss of response to, or intolerance of one or more of the agents.

In one aspect of A2 or A3, i.e., A4, the subject had an inadequate response to, loss of response to, or intolerance of no more than two classes of biologics.

In one aspect (Aspect 5; A5), provided herein is a method of treating ulcerative colitis or Crohn's disease in a subject in need thereof, the method comprising: (a) determining whether or not a subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, and a sphingosine-1-phosphate (S1P) receptor modulator; and (b) if the subject had an inadequate response, loss of response, or intolerance at least one of the agents in part (a), administering to the subject a composition comprising an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10.

In one aspect of A5, i.e., A6, the ulcerative colitis is moderate to severe ulcerative colitis and/or wherein the Crohn's disease is moderate to severe Crohn's disease.

In one aspect (Aspect; A7), provided herein is a method of treating ulcerative colitis or Crohn's disease in a subject in need thereof, the method comprising: (a) determining whether or not the subject has moderate to severe ulcerative colitis or moderate to severe Crohn's disease; and (b) if the subject has moderate to severe ulcerative colitis or moderate to severe Crohn's disease, administering to the subject a composition comprising an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10.

In one aspect of A7, i.e., A8, the subject was treated previously with one or more agents selected from the group consisting of a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA).

In one aspect of A8, i.e, A9, the subject had an inadequate response to, loss of response to, or intolerance of one or more of the agents.

In one aspect of A8 or A9, i.e., A10, the subject had an inadequate response to, loss of response to, or intolerance of no more than two classes of biologics.

In one aspect of any one of A1-A10, i.e., A11, the antibody or antigen-binding fragment of (i) or (ii) comprises an IgG1 constant region.

In one aspect of any one of A1-A11, i.e., A12, the subject is treated concurrently with an oral corticosteroid, an oral 5-ASA, sulfasalazine, azathioprine (AZA), 6-mercaptopurine (6-MP) and/or methotrexate.

In one aspect of any one of A1-A12, i.e., A13, the antibody or antigen-binding fragment thereof is administered at dose of about 300 mg.

In one aspect of any one of A1-A12, i.e., A14, the antibody or antigen-binding fragment thereof is administered at dose of about 450 mg.

In one aspect of any one of A1-A12, i.e., A15, the antibody or antigen-binding fragment thereof is administered at dose of about 600 mg.

In one aspect of any one of A1-A12, i.e., A16, the antibody or antigen-binding fragment thereof is administered at dose of about 750 mg.

In one aspect of any one of A1-A12, i.e., A17, the antibody or antigen-binding fragment thereof is administered at a dose of about 900 mg.

In one aspect of any one of A1-A12, i.e., A18, the antibody or antigen-binding fragment thereof is administered at dose of about 1500 mg.

In one aspect of any one of A1-A12, i.e., A19, the antibody or antigen-binding fragment thereof is administered at dose of about 1750 mg.

In one aspect of any one of A1-A12, i.e., A20, the antibody or antigen-binding fragment thereof is administered at a dose of about 1800 mg.

In one aspect of any one of A1-A12, i.e., A21, the antibody or antigen-binding fragment thereof is administered at dose of about 2000 mg.

In one aspect of any one of A1-A12, i.e., A22, the antibody or antigen-binding fragment thereof is administered at a dose of about 2250 mg.

In one aspect of any one of A1-A12, i.e., A23, the antibody or antigen-binding fragment thereof is administered at dose of about 2500 mg.

In one aspect of any one of A1-A23, i.e., A24, the antibody or antigen-binding fragment thereof is administered about once every two weeks.

In one aspect of any one of A1-A23, i.e., A25, the antibody or antigen-binding fragment thereof is administered about once a month.

In one aspect of any one of A1-A25, i.e., A26, the administration comprises administering one or more induction doses of the antibody or antigen-binding fragment thereof, and wherein no loading dose is administered.

In one aspect of any one of A1-A25, i.e., A27, the administration comprises administration of a loading dose of the antibody or antigen-binding fragment thereof, followed by administration of one or more induction doses of the antibody or antigen-binding fragment thereof.

In one aspect of A27, i.e., A28, the loading dose comprises about 1500 mg, about 1750 mg, about 2000 mg, about 2250 mg, or about 2500 mg of the antibody or antigen-binding fragment thereof.

In one aspect of A28, i.e., A29, the loading dose comprises about 2250 mg of the antibody or antigen-binding fragment thereof.

In one aspect of any one of A26-A29, i.e., A30, the one or more induction doses comprises about 450 mg of the antibody or antigen-binding fragment thereof.

In one aspect of any one of A26-A29, i.e., A31, the one or more induction doses comprises about 900 mg of the antibody or antigen-binding fragment thereof.

In one aspect of any one of A26-A29, i.e., A32, the one or more induction doses comprises about 1800 mg of the antibody or antigen-binding fragment thereof.

In one aspect of any one of A26-A32, i.e., A33, about six induction doses are administered.

In one aspect of any one of A1-A33, i.e., A34, the administration occurs over about 16 weeks.

In one aspect of any one of A1-A25, i.e., A35, the administration comprises administering one or more maintenance doses of the antibody or antigen-binding fragment thereof, and wherein no loading dose and/or induction dose is administered, optionally wherein the maintenance doses are administered about every 2 weeks.

In one aspect of any one of A1-A34, i.e., A35, the administration further comprises administering one or more maintenance doses of the antibody or antigen-binding fragment thereof, optionally wherein the maintenance doses are administered about every 2 weeks.

In one aspect of A35 or A36, i.e., A37, the one or more maintenance doses comprises about 300 mg or about 900 mg of the antibody or antigen-binding fragment thereof.

In one aspect of A37, i.e., A38, the one or more maintenance doses comprises about 300 mg of the antibody or antigen-binding fragment thereof.

In one aspect of A37, i.e., A39, the one or more maintenance doses comprises about 900 mg of the antibody or antigen-binding fragment thereof.

In one aspect of any one of A35-A39, i.e., A40, about twelve maintenance doses are administered.

In one aspect of any one of A1-A12, i.e., A41, the administration comprises administering (i) a loading dose of about 2250 mg, (ii) one or more induction doses of about 450 mg, and (iii) one or more maintenance doses of about 300 mg of the antibody or antigen-binding fragment thereof.

In one aspect of any one of A1-A12, i.e., A42, the administration comprises administering (i) a loading dose of about 2250 mg, (ii) one or more induction doses of about 900 mg, and (iii) one or more maintenance doses of about 300 mg of the antibody or antigen-binding fragment thereof.

In one aspect of any one of A1-A12, i.e., A43, the administration comprises administering (i) a loading dose of about 2250 mg, (ii) one or more induction doses of about 1800 mg, and (iii) one or more maintenance doses of about 300 mg of the antibody or antigen-binding fragment thereof.

In one aspect of any one of A1-A12, i.e., A44, the administration comprises administering (i) a loading dose of about 2250 mg, (ii) one or more induction doses of about 450 mg, and (iii) one or more maintenance doses of about 900 mg of the antibody or antigen-binding fragment thereof.

In one aspect of any one of A1-A12, i.e., A45, the administration comprises administering (i) a loading dose of about 2250 mg, (ii) one or more induction doses of about 900 mg, and (iii) one or more maintenance doses of about 900 mg of the antibody or antigen-binding fragment thereof.

In one aspect of any one of A1-A12, i.e., A46, the administration comprises administering (i) a loading dose of about 2250 mg, (ii) one or more induction doses of about 1800 mg, and (iii) one or more maintenance doses of about 900 mg of the antibody or antigen-binding fragment thereof.

In one aspect of any one of A27-A34 or A36-A46, i.e., A47, the administration of the one or more induction doses occurs over about 14 weeks.

In one aspect of any one of A27-A47, i.e., A48, the loading dose is administered about 2 weeks before the one or more induction doses are administered.

In one aspect of any one of A35-A48, i.e., A49, the administration of the one or more maintenance doses occurs over about 24 weeks.

In one aspect of any one of A1-A49, i.e., A50, the antibody or antigen-binding fragment thereof is formulated at a concentration of about 150 mg/mL.

In one aspect of any one of A1-A50, i.e., A51, the antibody or antigen-binding fragment thereof is formulated in a volume of 3 mL or less.

In one aspect of any one of A1-A51, i.e., A52, the antibody or antigen-binding fragment thereof is administered to the subject subcutaneously or intravenously.

In one aspect of A52, i.e., A53, the antibody or antigen-binding fragment thereof is administered subcutaneously.

In one aspect of any one of A1-A53, i.e., A54, the antibody or antigen-binding fragment thereof is administered to the subject via a syringe.

In one aspect of any one of A1-A54, i.e., A55, the subject has moderate to severe ulcerative colitis.

In one aspect of A55, i.e., A56, the administration results in clinical remission.

In one aspect of A56, i.e., A57, the clinical remission occurs within 14 weeks of the start of the administration.

In one aspect of A56, i.e., A58, the clinical remission occurs within 38 weeks of the start of the administration.

In one aspect of any one of A1-A54, i.e., A59, the subject has moderate to severe Crohn's disease.

In one aspect of A59, i.e., A60, the administration results in an endoscopic response.

In one aspect of A60, i.e., A61, the endoscopic response occurs within 14 weeks of the start of the administration.

In one aspect of A60, i.e., A62, the endoscopic response occurs within 38 weeks of the start of the administration.

In one aspect, i.e., A63, provided herein is a pharmaceutical formulation, comprising: (a) 150 mg/mL of an antibody or antigen-binding fragment thereof that specifically binds to TNF-like ligand 1A (TL1A); wherein the antibody or antigen-binding fragment thereof comprises: (i) a heavy chain variable region comprising the amino acid sequence of SEQ ID NO: 7 and a light chain variable region comprising the amino acid sequence of SEQ ID NO: 8; (ii) a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; or (iii) a heavy chain comprising the amino acid sequence of SEQ ID NO: 9 and a light chain comprising the amino acid sequence of SEQ ID NO: 10; (b) 10 mM Histidine; (c) 100 mM Arginine-Hydrochloride (Arg-HCl); (d) 5% (w/v) Sucrose; and (e) 0.02% (w/v) Polysorbate-80.

In one aspect of A63, i.e., A64, the antibody or antigen-binding fragment of (i) or (ii) comprises an IgG1 constant region.

In one aspect of A63 or A64, i.e., A65, the pharmaceutical formulation is lyophilized.

In one aspect of A63 or A64, i.e., A66, the pharmaceutical formulation is liquid.

In one aspect of any one of A63-A66, i.e., A67, the pharmaceutical composition has a pH is 6.0±0.5 after storage at room temperature for 24 hours or at 2-8° C. for 24 hours, 72 hours, or 10 days.

In one aspect of any one of A63-A67, i.e., A68, the pharmaceutical composition has an osmolality from 200 mOsm/kg to 500 mOsm/kg after storage at room temperature for 24 hours or at 2-8° C. for 24 hours, 72 hours, or 10 days.

In one aspect of any one of A63-A68, i.e., A69, the pharmaceutical composition has at least 99% antibody monomer content after storage at 2-8° C. for 24 hours, 72 hours, or 10 days.

In one aspect of any one of A63-A69, i.e., A70, the pharmaceutical composition has no significant change in charge heterogeneity profile after storage at 2-8° C. for 24 hours, 72 hours, or 10 days.

In one aspect of any one of A63-A70, i.e., A71, the pharmaceutical composition has no significant change in purity after storage at room temperature for 24 hours or at 2-8° C. for 24 hours, 72 hours, or 10 days.

In one aspect of any one of A63-A70, i.e. A72, the pharmaceutical composition has at least 90% purity after storage at room temperature for 24 hours or at 2-8° C. for 24 hours, 72 hours, or 10 days.

In one aspect of any one of A63-A72, i.e., A73, the pharmaceutical composition has no significant change in particle concentration after storage at room temperature for 24 hours or at 2-8° C. for 24 hours, 72 hours, or 10 days.

In one aspect of any one of A63-A73, i.e., A74, the pharmaceutical composition has no significant difference in visual appearance after storage at 2-8° C. for up to 36 months.

In one aspect of any one of A63-A74, i.e., A75, the pharmaceutical composition has no significant difference in protein concentration, osmolality or viscosity after storage at 2-8° C., 25° C. or 40° C. for up to 36 months.

In one aspect of any one of A63-A75, i.e., A76, the pharmaceutical composition has ≥95% monomer content, ≤5.0% dimer content, or no significant difference in low molecular weight species content after storage at 2-8° C. for up to 36 months.

In one aspect of any one of A63-A76, i.e., A77, the pharmaceutical composition has ≥90% purity after storage at 2-8° C. for up to 36 months.

In one aspect of any one of A63-A77, i.e., A78, the pharmaceutical composition has from 50% to 90% main species content, from 10% to 40% acidic species content, and/or from 0% to 20% basic species content after storage at 2-8° C. for up to 36 months.

In one aspect of any one of A63-A78, i.e., A79, the pharmaceutical composition has no significant difference in sub-visible particle content after storage at 2-8° C., 25° C., or 40° C. for up to 36 months.

In one aspect of any one of A63-A79, i.e., A80, the pharmaceutical composition has no significant difference in oxidation of methionine 81 and/or methionine 254 of TEV-48574, and/or deamidation of asparagine 317 of TEV-48574 after storage at 2-8° C., 25° C., or 40° C. for up to 36 months.

In one aspect of any one of A63-A80, i.e., A81, the pharmaceutical composition has from 70% to 135% relative potency measured by enzyme-linked immunosorbent assay (ELISA) after storage at 2-8° C. for up to 36 months.

In one aspect of any one of A63-A81, i.e., A82, the pharmaceutical composition has no significant difference in thermal stability after storage at 2-8° C., 25° C., or 40° C. for up to 6 months.

In one aspect of any one of A63-A82, i.e., A83, the pharmaceutical composition has no significant difference in thermal stability after storage at 2-8° C. for up to 36 months.

In one aspect of any one of A63-A83, i.e., A84, the pharmaceutical composition has no significant difference in secondary and/or tertiary protein structure after storage at 2-8° C., 25° C., or 40° C. for up to 3 months.

In one aspect of any one of A63-A84, i.e., A85, the pharmaceutical composition has no significant difference in secondary protein structure after storage at 2-8° C. for up to 36 months.

In one aspect of any one of A63-A85, i.e., A86, the pharmaceutical composition has no significant difference in concentration of Polysorbate-80 after storage at 2-8° C. for up to 24 months. In one aspect, i.e., A87, a container is provided comprising the pharmaceutical formulation of any one of A63-A86.

In one aspect of A87, i.e., A88, the container is a glass vial.

In one aspect of A88, i.e., A89, the container is a glass vial having a fill volume of 3 mL.

In one aspect of any one of A1-A62, i.e., A90, the antibody or antigen-binding fragment thereof is present in the pharmaceutical formulation of any one of A63-A86, or the container of any one of A87-A89.

In one aspect, i.e., A91, a composition for use in accordance with the method of any one of A1-A62 or A90 is provided herein.

In one aspect of the method of any one of A1-A62 or A90, the pharmaceutical formulation of any one of A63-A86, or the container of any one of A87-A89, i.e., A92, the antibody or antigen-binding fragment thereof was produced in a Chinese hamster ovary cell.

In one aspect, i.e., B1, provided herein is a method of treating moderate to severe ulcerative colitis or moderate to severe Crohn's disease in a subject in need thereof, comprising administering to the subject an antibody or antigen-binding fragment thereof that specifically binds to TNF-like ligand 1A (TL1A); wherein the antibody or antigen-binding fragment comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6.

In one aspect of B1, i.e., B2, the subject was treated previously with one or more agents selected from the group consisting of a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, a sphingosine-1-phosphate (S1P) receptor modulator, and 5-aminosalicylic acid (5-ASA).

In one aspect of B2, i.e., B3, the subject had an inadequate response to, loss of response to, or intolerance of one or more of the agents.

In one aspect of B2 or B3, i.e., B4, the subject had an inadequate response to, loss of response to, or intolerance of no more than two classes of biologics.

In one aspect, i.e., B5, provided herein is a method of treating ulcerative colitis or Crohn's disease in a subject in need thereof, the method comprising: (a) determining whether or not a subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti interleukin (IL)-12/23 antibody, a Janus kinase (JAK) inhibitor, and a sphingosine-1-phosphate (S1P) receptor modulator; and (b) if the subject had an inadequate response, loss of response, or intolerance at least one of the agents in part (a), administering to the subject a composition comprising an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6.

In one aspect of B5, i.e., B6, the ulcerative colitis is moderate to severe ulcerative colitis and/or wherein the Crohn's disease is moderate to severe Crohn's disease.

In one aspect, i.e., B7, provided herein is a method of treating ulcerative colitis or Crohn's disease in a subject in need thereof, the method comprising: (a) determining whether or not the subject has moderate to severe ulcerative colitis or moderate to severe Crohn's disease; and (b) if the subject has moderate to severe ulcerative colitis or moderate to severe Crohn's disease, administering to the subject a composition comprising an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6.

In one aspect of B7, i.e., B8, the subject was treated previously with one or more agents selected from the group consisting of a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA).

In one aspect of B8, i.e, B9, the subject had an inadequate response to, loss of response to, or intolerance of one or more of the agents.

In one aspect of B8 or B9, i.e., B10, the subject had an inadequate response to, loss of response to, or intolerance of no more than two classes of biologics.

In one aspect of any one of B1-B10, i.e., B11, the antibody or antigen-binding fragment comprises a heavy chain variable region comprising the amino acid sequence of SEQ ID NO: 7 and a light chain variable region comprising the amino acid sequence of SEQ ID NO: 8.

In one aspect of any one of B1-B10, i.e., B12, the antibody or antigen-binding fragment comprises an IgG1 constant region.

In one aspect of any one of B1-B12, i.e., B13, the antibody or antigen-binding fragment comprises a heavy chain comprising the amino acid sequence of SEQ ID NO: 9 and a light chain comprising the amino acid sequence of SEQ ID NO: 10.

In one aspect of any one of B1-B13, i.e., B14, the subject is treated concurrently with an oral corticosteroid, an oral 5-ASA, sulfasalazine, azathioprine (AZA), 6-mercaptopurine (6-MP) and/or methotrexate.

In one aspect of any one of B1-B14, i.e., B15, the antibody or antigen-binding fragment thereof is administered at dose of about 300 mg.

In one aspect of any one of B1-B14, i.e., B16, the antibody or antigen-binding fragment thereof is administered at dose of about 400 mg.

In one aspect of any one of B1-B14, i.e., B17, the antibody or antigen-binding fragment thereof is administered at dose of about 450 mg.

In one aspect of any one of B1-B14, i.e., B18, the antibody or antigen-binding fragment thereof is administered at dose of about 600 mg.

In one aspect of any one of B1-B14, i.e., B19, the antibody or antigen-binding fragment thereof is administered at dose of about 750 mg.

In one aspect of any one of B1-B14, i.e., B20, the antibody or antigen-binding fragment thereof is administered at a dose of about 900 mg.

In one aspect of any one of B1-B14, i.e., B21, the antibody or antigen-binding fragment thereof is administered at a dose of about 2250 mg.

In one aspect of any one of B1-B20, i.e., B22, the antibody or antigen-binding fragment thereof is administered about once every two weeks.

In one aspect of any one of B1-B20, i.e., B23, the antibody or antigen-binding fragment thereof is administered about once a month.

In one aspect of any one of B1-B23, i.e., B24, the administration comprises administering one or more induction doses of the antibody or antigen-binding fragment thereof, and wherein no loading dose is administered.

In one aspect of any one of B1-B23, i.e., B25, the administration comprises administration of a loading dose of the antibody or antigen-binding fragment thereof, followed by administration of one or more induction doses of the antibody or antigen-binding fragment thereof.

In one aspect of B25, i.e., B26, the loading dose comprises about 2250 mg of the antibody or antigen-binding fragment thereof.

In one aspect of any one of B24-B26, i.e., B27, the one or more induction doses comprises about 450 mg of the antibody or antigen-binding fragment thereof.

In one aspect of any one of B24-B26, i.e., B28, the one or more induction doses comprises about 900 mg of the antibody or antigen-binding fragment thereof.

In one aspect of any one of B24-B28, i.e., B29, about six induction doses are administered.

In one aspect of any one of B1-B29, i.e., B30, the administration occurs over about 16 weeks.

In one aspect of any one of B1-B23, i.e., B31, the administration comprises administering one or more maintenance doses of the antibody or antigen-binding fragment thereof, and wherein no loading dose and/or induction dose is administered, optionally wherein the maintenance doses are administered about every 2 weeks.

In one aspect of any one of B1-B30, i.e., B32, the administration further comprises administering one or more maintenance doses of the antibody or antigen-binding fragment thereof, optionally wherein the maintenance doses are administered about every 2 weeks.

In one aspect of any one of B1-B23, i.e., B33, the administration comprises administering one or more maintenance doses of the antibody or antigen-binding fragment thereof, and wherein no loading dose and/or induction dose is administered, optionally wherein the maintenance doses are administered about every 4 weeks.

In one aspect of any one of B1-B23, i.e., B34, the administration further comprises administering one or more maintenance doses of the antibody or antigen-binding fragment thereof, optionally wherein the maintenance doses are administered about every 4 weeks.

In one aspect of any one of B30-B34, i.e., B35, the one or more maintenance doses comprises about 450 mg or about 900 mg of the antibody or antigen-binding fragment thereof.

In one aspect of B35, i.e., B36, the one or more maintenance doses comprises about 450 mg of the antibody or antigen-binding fragment thereof.

In one aspect of B35, i.e., B37, the one or more maintenance doses comprises about 900 mg of the antibody or antigen-binding fragment thereof.

In one aspect of any one of B30-B37, i.e., B38, about ten maintenance doses are administered.

In one aspect of any one of B1-B14, i.e., B39, the administration comprises administering (i) a loading dose of about 2250 mg, (ii) one or more induction doses of about 450 mg, and (iii) one or more maintenance doses of about 450 mg of the antibody or antigen-binding fragment thereof, optionally wherein the induction doses are administered about every two weeks and/or the maintenance doses are administered about every four weeks.

In one aspect of any one of B1-B14, i.e., B40, the administration comprises administering (i) a loading dose of about 2250 mg, (ii) one or more induction doses of about 900 mg, and (iii) one or more maintenance doses of about 450 mg of the antibody or antigen-binding fragment thereof, optionally wherein the induction doses are administered about every two weeks and/or the maintenance doses are administered about every four weeks.

In one aspect of any one of B1-B14, i.e., B41, the administration comprises administering (i) a loading dose of about 2250 mg, (ii) one or more induction doses of about 450 mg, and (iii) one or more maintenance doses of about 900 mg of the antibody or antigen-binding fragment thereof, optionally wherein the induction doses are administered about every two weeks and/or the maintenance doses are administered about every four weeks.

In one aspect of any one of B1-B14, i.e., B42, the administration comprises administering (i) a loading dose of about 2250 mg, (ii) one or more induction doses of about 900 mg, and (iii) one or more maintenance doses of about 900 mg of the antibody or antigen-binding fragment thereof, optionally wherein the induction doses are administered about every two weeks and/or the maintenance doses are administered about every four weeks.

In one aspect of any one of B39-B42, i.e., B43, about 6 induction doses are administered and/or about ten maintenance doses are administered.

In one aspect of any one of B1-B14, i.e., B44, the administration comprises administering (i) a loading dose of about 2250 mg and (ii) one or more maintenance doses of about 450 mg of the antibody or antigen-binding fragment thereof, optionally wherein the maintenance doses are administered about every four weeks.

In one aspect of any one of B1-B14, i.e., B45, the administration comprises administering (i) a loading dose of about 2250 mg and (ii) one or more maintenance doses of about 900 mg of the antibody or antigen-binding fragment thereof, optionally wherein the maintenance doses are administered about every four weeks.

In one aspect of B44 or B45, i.e., B46, at least ten maintenance doses are administered.

In one aspect of any one of B24-B46, i.e., B47, the administration of the one or more induction doses occurs over about 14 weeks.

In one aspect of any one of B25-B47, i.e., B48, the loading dose is administered about 2 weeks before the one or more induction doses are administered.

In one aspect of any one of B31-B48, i.e., B49, the administration of the one or more maintenance doses occurs over about 24 weeks.

In one aspect of any one of B31-B48, i.e., B50, the administration of the one or more maintenance doses occurs over about 40 weeks.

In one aspect of any one of B1-B50, i.e., B51, the antibody or antigen-binding fragment thereof is formulated at a concentration of about 150 mg/mL.

In one aspect of any one of B1-B50, i.e., B52, the antibody or antigen-binding fragment thereof is formulated at a concentration of about 200 mg/mL.

In one aspect of any one of B1-B52, i.e., B53, the antibody or antigen-binding fragment thereof is formulated in a volume of 3 mL or less.

In one aspect of any one of B1-B53, i.e., B54, the antibody or antigen-binding fragment thereof is administered to the subject subcutaneously or intravenously.

In one aspect of B54, i.e., B55, the antibody or antigen-binding fragment thereof is administered subcutaneously.

In one aspect of any one of B1-B55, i.e., B56, the antibody or antigen-binding fragment thereof is administered to the subject via a syringe.

In one aspect of B56, i.e., B57, the syringe is a pre-filled syringe.

In one aspect of any one of B1-B57, i.e., B58, the subject has moderate to severe ulcerative colitis.

In one aspect of B58, i.e., B59, the administration results in clinical remission.

In one aspect of B59, i.e., B60, the clinical remission occurs within 14 weeks of the start of the administration.

In one aspect of B59, i.e., B61, the clinical remission occurs within 38 weeks of the start of the administration.

In one aspect of any one of B1-B57, i.e., B62, the subject has moderate to severe Crohn's disease.

In one aspect of B62, i.e., B63, the administration results in an endoscopic response.

In one aspect of B63, i.e., B64, the endoscopic response occurs within 14 weeks of the start of the administration.

In one aspect of B64, i.e., B65, the endoscopic response occurs within 38 weeks of the start of the administration.

In one aspect, i.e., B66, an antibody or antigen-binding fragment thereof that specifically binds to TNF-like ligand 1A (TL1A) for use in the preparation of a medicament of any one of B1-B65.

In one aspect, i.e., B67, an antibody or antigen-binding fragment thereof that specifically binds to TNF-like ligand 1A (TL1A) for use in any one of B1-B65.

In one aspect, i.e., B68, provided herein is a pharmaceutical formulation, comprising: (a) about 100 mg/mL to about 250 mg/mL of an antibody or antigen-binding fragment thereof that specifically binds to TNF-like ligand 1A (TL1A); wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; (b) about 5 mM to about 15 mM Histidine; (c) about 50 mM to about 150 mM Arginine-Hydrochloride (Arg-HCl); (d) about 2.5% (w/v) to about 7.5% (w/v) Sucrose; and (e) about 0.01% (w/v) to about 0.03% (w/v) Polysorbate-80.

In one aspect of B68, i.e., B69, the antibody or antigen-binding fragment thereof comprises a heavy chain variable region comprising the amino acid sequence of SEQ ID NO: 7 and a light chain variable region comprising the amino acid sequence of SEQ ID NO: 8.

In one aspect of B68 or B69, i.e., B70, the antibody or antigen-binding fragment comprises an IgG1 constant region.

In one aspect of any one of B68-B70, i.e., B71, the antibody or antigen-binding fragment thereof comprises a heavy chain comprising the amino acid sequence of SEQ ID NO: 9 and a light chain comprising the amino acid sequence of SEQ ID NO: 10.

In one aspect of any one of B68-B71, i.e., B72, the pharmaceutical formulation comprises about 100 mg/mL of the antibody or antigen-binding fragment thereof.

In one aspect of any one of B68-B71, i.e., B73, the pharmaceutical formulation comprises about 150 mg/mL of the antibody or antigen-binding fragment thereof.

In one aspect of any one of B68-B71, i.e., B74, the pharmaceutical formulation comprises about 200 mg/mL of the antibody or antigen-binding fragment thereof.

In one aspect of any one of B68-B71, i.e., B75, the pharmaceutical formulation comprises about 225 mg/mL of the antibody or antigen-binding fragment thereof.

In one aspect of any one of B68-B71, i.e., B76, the pharmaceutical formulation comprises about 250 mg/mL of the antibody or antigen-binding fragment thereof.

In one aspect of any one of B68-B76, i.e., B77, the pharmaceutical formulation comprises about 5 mM Histidine.

In one aspect of any one of B68-B76, i.e., B78, the pharmaceutical formulation comprises about 10 mM Histidine.

In one aspect of any one of B68-B76, i.e., B79, the pharmaceutical formulation comprises about 15 mM Histidine.

In one aspect of any one of B68-B79, i.e., B80, the pharmaceutical formulation comprises about 50 mM Arginine-Hydrochloride (Arg-HCl).

In one aspect of any one of B68-B79, i.e., B81, the pharmaceutical formulation comprises about 100 mM Arginine-Hydrochloride (Arg-HCl).

In one aspect of any one of B68-B79, i.e., B82, the pharmaceutical formulation comprises about 150 mM Arginine-Hydrochloride (Arg-HCl).

In one aspect of any one of B68-B82, i.e., B83, the pharmaceutical formulation comprises about 2.5% (w/v) Sucrose.

In one aspect of any one of B68-B82, i.e., B84, the pharmaceutical formulation comprises about 5% (w/v) Sucrose.

In one aspect of any one of B68-B82, i.e., B85, the pharmaceutical formulation comprises about 7.5% (w/v) Sucrose.

In one aspect of any one of B68-B85, i.e., B86, the pharmaceutical formulation comprises about 0.01% (w/v) Polysorbate-80.

In one aspect of any one of B68-B85, i.e., B87, the pharmaceutical formulation comprises about 0.02% (w/v) Polysorbate-80.

In one aspect of any one of B68-B85, i.e., B88, the pharmaceutical formulation comprises about 0.03% (w/v) Polysorbate-80.

In one aspect of any one of B68-B71, i.e., B89, the pharmaceutical formulation comprises about 250 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In one aspect of any one of B68-B71, i.e., B90, the pharmaceutical formulation comprises about 200 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In one aspect of any one of B68-B71, i.e., B91, the pharmaceutical formulation comprises about 150 mg/mL of the antibody or antigen binding fragment thereof, about 10 mM Histidine, about 100 mM Arginine-Hydrochloride (Arg-HCl), about 5% (w/v) Sucrose, and about 0.02% (w/v) Polysorbate-80.

In one aspect of any one of B68-B91, i.e., B92, the pharmaceutical formulation is lyophilized.

In one aspect of any one of B68-B91, i.e., B93, the pharmaceutical formulation is liquid.

In one aspect of any one of B68-B93, i.e., B94, the pharmaceutical composition has a pH is 6.0±0.5 after storage at room temperature for 24 hours or at 2-8° C. for 24 hours, 72 hours, or 10 days.

In one aspect of any one of B68-B94, i.e., B95, the pharmaceutical composition has an osmolality from 200 mOsm/kg to 500 mOsm/kg after storage at room temperature for 24 hours or at 2-8° C. for 24 hours, 72 hours, or 10 days.

In one aspect of any one of B68-B95, i.e., B96, the pharmaceutical composition has at least 99% antibody monomer content after storage at 2-8° C. for 24 hours, 72 hours, or 10 days.

In one aspect of any one of B68-B96, i.e., B97, the pharmaceutical composition has no significant change in charge heterogeneity profile after storage at 2-8° C. for 24 hours, 72 hours, or 10 days.

In one aspect of any one of B68-B97, i.e., B98, the pharmaceutical composition has no significant change in purity after storage at room temperature for 24 hours or at 2-8° C. for 24 hours, 72 hours, or 10 days.

In one aspect of any one of B68-B98, i.e. B99, the pharmaceutical composition has at least 90% purity after storage at room temperature for 24 hours or at 2-8° C. for 24 hours, 72 hours, or 10 days.

In one aspect of any one of B68-B99, i.e., B100, the pharmaceutical composition has no significant change in particle concentration after storage at room temperature for 24 hours or at 2-8° C. for 24 hours, 72 hours, or 10 days.

In one aspect of any one of B68-B100, i.e., B101, the pharmaceutical composition has no significant difference in visual appearance after storage at 2-8° C. for up to 36 months.

In one aspect of any one of B68-B101, i.e., B102, the pharmaceutical composition has no significant difference in protein concentration, osmolality or viscosity after storage at 2-8° C., 25° C. or 40° C. for up to 36 months.

In one aspect of any one of B68-B102, i.e., B103, the pharmaceutical composition has ≥95% monomer content, ≤5.0% dimer content, or no significant difference in low molecular weight species content after storage at 2-8° C. for up to 36 months.

In one aspect of any one of B68-B103, i.e., B104, the pharmaceutical composition has ≥90% purity after storage at 2-8° C. for up to 36 months.

In one aspect of any one of B68-B104, i.e., B105, the pharmaceutical composition has from 50% to 90% main species content, from 10% to 40% acidic species content, and/or from 0% to 20% basic species content after storage at 2-8° C. for up to 36 months.

In one aspect of any one of B68-B105, i.e., B106, the pharmaceutical composition has no significant difference in sub-visible particle content after storage at 2-8° C., 25° C., or 40° C. for up to 36 months.

In one aspect of any one of B68-B106, i.e., B107, the pharmaceutical composition has no significant difference in oxidation of methionine 81 and/or methionine 254 of TEV-48574, and/or deamidation of asparagine 317 of TEV-48574 after storage at 2-8° C., 25° C., or 40° C. for up to 36 months.

In one aspect of any one of B68-B107, i.e., B108, the pharmaceutical composition has from 70% to 135% relative potency measured by enzyme-linked immunosorbent assay (ELISA) after storage at 2-8° C. for up to 36 months.

In one aspect of any one of B68-B108, i.e., B109, the pharmaceutical composition has no significant difference in thermal stability after storage at 2-8° C., 25° C., or 40° C. for up to 6 months.

In one aspect of any one of B68-B109, i.e., B110, the pharmaceutical composition has no significant difference in thermal stability after storage at 2-8° C. for up to 36 months.

In one aspect of any one of B68-B110, i.e., B111, the pharmaceutical composition has no significant difference in secondary and/or tertiary protein structure after storage at 2-8° C., 25° C., or 40° C. for up to 3 months.

In one aspect of any one of B68-B111, i.e., B112, the pharmaceutical composition has no significant difference in secondary protein structure after storage at 2-8° C. for up to 36 months.

In one aspect of any one of B68-B112, i.e., B113, the pharmaceutical composition has no significant difference in concentration of Polysorbate-80 after storage at 2-8° C. for up to 24 months.

In one aspect of any one of B68-B113, i.e., B114, the antibody or antigen-binding fragment thereof was produced in a Chinese hamster ovary cell.

In one aspect, i.e., B115, a container is provided comprising the pharmaceutical formulation of any one of B68-B114.

In one aspect of B115, i.e., B116, the container is a glass vial.

In one aspect of B116, i.e., B117, the container is a glass vial having a fill volume of 3 mL.

In one aspect of B115, i.e., B118, the container is a syringe, optionally wherein the syringe is a pre-filled syringe.

In one aspect, i.e., B119, provided herein is a method of treating a disease in a subject in need thereof, the method comprising administering to the subject the pharmaceutical formulation of any one of B68-B114 or the container of any one of B115-B118, optionally wherein the disease is a gastrointestinal disease.

In one aspect of B119, i.e., B120, the gastrointestinal disease is Crohn's disease or ulcerative colitis.

In one aspect of B119 or B120, i.e., B121, the pharmaceutical formulation is administered intravenously.

In one aspect of B119 or B120, i.e., B122, the pharmaceutical formulation is administered subcutaneously.

In one aspect of any one of B68-B114, i.e., B123, the formulation is for use in accordance with the method of any one of B1-B65 or B115-B122.

The following examples are offered by way of illustration and not by way of limitation.

### EXAMPLES

### Example 1: Clinical Evaluation of TEV-48574 in Moderate to Severe Ulcerative Colitis or Moderate to Severe Crohn's Disease

TEV-48574 is assessed in a Phase 2b, randomized, double-blind, dose-ranging study to determine the pharmacokinetics, efficacy, safety, and tolerability of TEV-48574 in adult patients with moderate to severe ulcerative colitis (UC) or Crohn's disease (CD). The study enrolls adult patients (18 to 75 years of age, inclusive) of male and female sex (without restrictions on gender) with moderate to severe active UC or CD and who have demonstrated an inadequate response to, loss of response to, or intolerance of at least 1 of the following agents and no more than 2 classes of biologics: a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA).

The study demonstrates the efficacy and dose response of 3 different dose regimens of TEV-48574 subcutaneously (sc) administered every 2 weeks (Q2W) in adult patients with inflammatory bowel disease (IBD) (moderate to severe UC or CD) as assessed by induction of clinical remission (UC) and endoscopic response (CD) at week 14.

A single loading dose of 2250 mg is used in this study. The loading dose as part of the induction dose regimen has been selected to rapidly achieve steady-state concentration and address the significant over-production of TL1A in the colon within the first weeks of induction.

The dosing regimen is designed to achieve steady state-concentration quickly. TEV-48574 has linear PK for the dose range of 200 to 2300 mg with elimination half-life of 7-9 days. A 2300 mg loading dose followed by 1600 mg doses reached steady-state after 2 weeks and no accumulation was observed. Population PK approaches and models were used to simulate the loading and induction doses and therefore predict resulting exposures. The 3 induction dose strengths in this study were selected based on a combination of safety, preclinical evidence, pharmacokinetic, immunogenic, and clinical considerations.

### Subject Population and Eligibility Criteria

Approximately 570 patients are screened to achieve approximately 280 randomized patients (approximately 140 patients with UC and 140 patients with CD).

Eligible patients meet all of the following criteria:
a. Adults of male and female sex (without restrictions on gender) between 18 and 75 years of age, inclusive, at the time of informed consent.
b. Diagnosis of UC or CD for ≥ (more than or equal to) 3 months.
c. UC patients only: Patient with moderate to severe active UC as defined by the 3-component modified Mayo score of 5 to 9, inclusive, with an endoscopic subscore of ≥ (more than or equal to) 2 (from central reading)
d. CD patients only: Patient with moderate to severe active CD as determined by a CDAI score of ≥ (more than or equal to) 220 and ≤ (less than or equal to) 450.
e. CD patients only: SES CD score of ≥ (more than or equal to) 6 (≥ (more than or equal to) 4 for isolated ileal disease).
f. UC patients only: Active disease beyond the rectum (>15 cm of active disease at the screening endoscopy [sigmoidoscopy]).
g. Patient must have inadequate response to, loss of response to, or intolerance of at least 1 of the following agents and no more than 2 classes of biologics: corticosteroids, immunosuppressant drugs, and/or TNF-α antagonist therapy, anti-integrins, anti-IL-12/23, JAK inhibitors, and/or SIP receptor modulators.
h. If patient is taking the following agents, patient must have been on a stable dose for the following specified period of time: oral 5-ASA or sulfasalazine stable dose for at least 4 weeks prior to endoscopy, oral corticosteroids stable dose for at least 2 weeks prior to endoscopy, and 6-MP, AZA, or methotrexate stable dose for 4 weeks prior to endoscopy.

Patients who meet all the inclusion criteria are randomly assigned to receive TEV-48574 (single loading dose/6 induction doses): 2250/1800 mg, 2250/900 mg, 2250/450 mg, or placebo to match TEV-48574, in a 1:1:1:1 ratio, stratified by diagnosis (UC or CD) and previous exposure, failure of biologics and small molecules (including JAK inhibitors and SIP receptor modulators for UC) or naive to biologics and small molecule therapy.

### Drug Dose, Mode of Administration, and Administration Rate

TEV-48574 for subcutaneous (sc) injection is provided as a liquid solution with a concentration of 150 mg/mL. Placebo is provided as a liquid solution in the same formulation as the TEV-48574, except for absence of active protein. Specific details regarding TEV-48574 and placebo products are provided in Table 1.

TABLE 1
TEV-48574 and Placebo Products

| | TEV-48574 | Placebo |
|---|---|---|
| Formulation | 10 mM histidine, 5% | 10 mM histidine, 5% |
| | (w/v) sucrose, 100 mM | (w/v) sucrose, 100 mM |
| | arginine-hydrochloride, | arginine-hydrochloride, |
| | 0.02% (w/v) | 0.02% (w/v) |
| | polysorbate-80, pH 6.0. | polysorbate-80, pH 6.0. |
| | Liquid. Protein | Liquid absent of protein |
| | concentration: | |
| | 150 mg/mL | |
| Unit dose | 450 mg/vial | — |
| strength/Dosage | | |
| level | | |
| Route of | Subcutaneous | Subcutaneous |
| Administration | administration | administration |

Patients receive the following regimens as a SC administration using a commercial syringe-based SC infusion system:
TEV-48574 2250 mg (single loading dose)/1800 mg (6 induction doses)
TEV-48574 2250 mg (single loading dose)/900 mg (6 induction doses)
TEV-48574 2250 mg (single loading dose)/450 mg (6 induction doses)
Placebo to match TEV-48574

The patients receive the loading dose on the day of randomization and the subsequent corresponding maintenance dose every 2 weeks for a total of 7 doses (1 loading and 6 induction doses). Administration is by subcutaneous (sc) infusion.

The long-term efficacy and safety of TEV-48574 will be demonstrated in patients who had moderately to severely active UC or CD at the beginning of the study and who also showed a clinical response and/or clinical remission (based on complete Mayo score for the UC and CDAI for the CD subjects) to initial treatment with TEV-48574 in the study. These patients will receive either 300 mg Q2W or 900 mg Q2W for the duration of the administration.

The commercial syringe infusion systems used for administration are capable of SC drug administration of up to 60 mL syringe using a SC safety needle set. The system controls the rate of drug entry into the tissues surrounding the SC infusion site as well as the duration of infusion. The syringe infusion system is a mechanical or electromechanical syringe pump that pushes the syringe plunger at a constant rate throughout the infusion to infuse drug.

### General Design

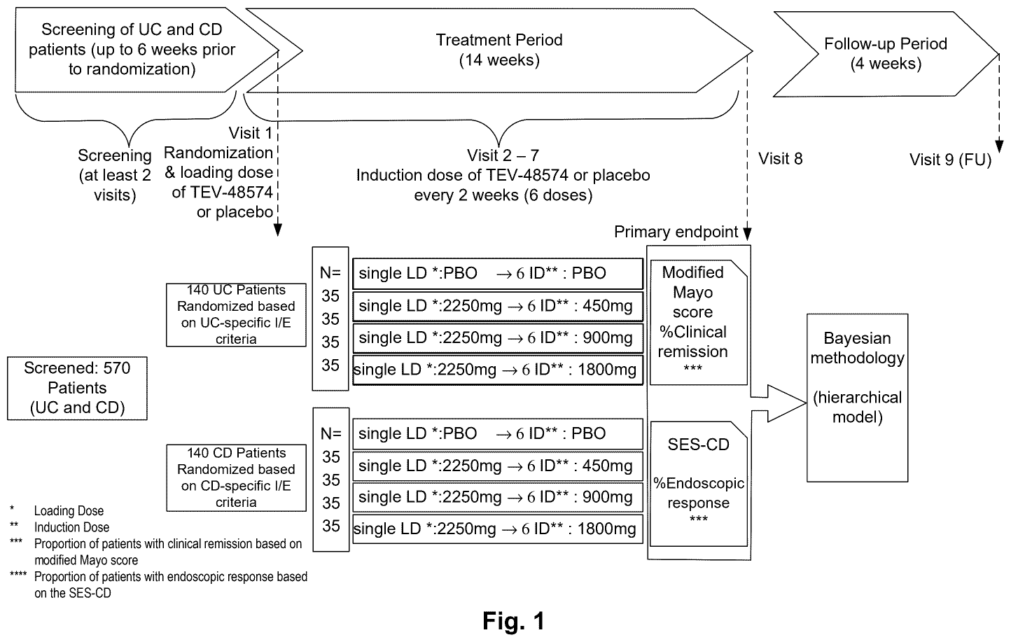

The study consists of a screening period of up to 6 weeks (42 days), a 14-week treatment period, and a 4-week follow-up period. The study schematic diagram is presented in FIG. 1.

Screening: Patients are screened within 6 weeks (42 days) prior to randomization to confirm that they have met all the selection criteria for the study. An endoscopy is performed after key eligibility criteria have been met and within approximately 10 calendar days of randomization (day 1) to allow for central endoscopy scoring.

Randomization: Patients satisfying the eligibility criteria at the end of the screening period are randomized in a 1:1:1:1 ratio (stratified by diagnosis [UC or CD] and previous exposure, failure of biologics and small molecules [including JAK inhibitors and SIP receptor modulators for UC] or naive to biologics and small molecule therapy) to 1 of 4 treatment groups for the double-blind treatment period (Table 2).

All treatments are administered in a double-blind fashion as 1 SC infusion Q2W.

TABLE 2
Randomization Scheme

| Randomization | Planned approximate | |
|---|---|---|
| ratio | number randomized | Treatment (Q2W) |
| 1 | 35 UC + 35 CD | TEV-48574 2250 mg (single |
| | | loading dose)/1800 mg |
| | | (6 induction doses) |
| 1 | 35 UC + 35 CD | TEV-48574 2250 mg (single |
| | | loading dose)/900 mg |
| | | (6 induction doses) |
| 1 | 35 UC + 35 CD | TEV-48574 2250 mg (single |
| | | loading dose)/450 mg |
| | | (6 induction doses) |
| 1 | 35 UC + 35 CD | Placebo |

CD = Crohn's disease; Q2W = every 2 weeks; UC = ulcerative colitis.

During the 14-week treatment period, patients will visit the site Q2W on days 1, 15, 29, 43, 57, 71, and 85 (±3 days) for TEV-48574/placebo administration (7 visits), as well as an end-of-treatment visit on day 99 (±3 days; week 14). The end of study is defined as the last visit of the last patient. The study duration is approximately 24 months.

The following efficacy assessments are performed. Endoscopy and histology assessments will be scored centrally.

### Modified Mayo Score (Ulcerative Colitis)

The modified Mayo score evaluates UC stage based on the following 3 parameters:
stool frequency
rectal bleeding
endoscopic evaluation

Each parameter of the score ranges from 0 (normal or inactive disease) to 3 (severe activity) and the total score from 0 to 9, respectively (Ulcerative Colitis: Clinical Trial Endpoints Guidance for Industry 2016, Naegeli et al., 2018).

### Endoscopy

Endoscopy is performed at screening and week 14 or the early termination visit. Assessments and procedures performed during the endoscopy, including biopsy collection, SES-CD, and modified multiplier (MM)-SES-CD, are described below.

### Biopsy Collection

For patients with CD, an ileo-colonoscopy is performed at screening and week 14. During each endoscopy, a total of 16 to 18 mucosal biopsies are collected and handled as outlined below. Mucosal biopsies are obtained from the area with the greatest inflammation in each segment. If ulceration is present, biopsies are taken from the edge of the largest ulcer. If no ulceration is present, then biopsies are taken from the most affected area of the segment. If the mucosa appears normal (eg, at follow-up), then random biopsies of the segment are obtained.

For patients with UC, a flexible sigmoidoscopy is performed at screening and week 14 (colonoscopy can be performed instead for baseline endoscopy if not done in the prior 12 months). During each endoscopy, a total of 8 to 12 biopsies are obtained from the area with the worst disease 15 to 25 cm from the anal verge. If ulceration is present, biopsies are taken from the edge of the largest ulcer. If no ulceration is present, then biopsy is taken from the most affected area. If mucosa appears normal (e.g., at follow-up), then random biopsies are taken from the area 15 to 25 cm from the anal verge.

Biopsies are used for measures of histologic disease.

### Simple Endoscopic Score for Crohn's Disease

The SES-CD takes into account 4 parameters (presence of ulcers, percentage of ulcerated surfaces, affected surface, and presence of strictures) that need to be scored in 5 bowel segments (the rectum, sigmoid and left colon, transverse colon, right colon, and ileum) (Daperno et al., Gastrointest Endosc; 60 (4): 505-12 (2004)).

### Modified Multiplier-Simple Endoscopic Score for Crohn's Disease

The MM-SES-CD is an endoscopic scoring tool, which takes into consideration each individual parameter's prognostic value for achieving endoscopic remission while on active therapy (Narula et al., Gut, 0:1-10 (2021)).

### Robarts Histopathology Index (Ulcerative Colitis)

The Robarts Histopathology Index (RHI) final score is obtained by combining the subscores of 4 main items:
chronic inflammatory infiltrate level (4 levels)
lamina propria neutrophils (4 levels)
neutrophils in the epithelium (4 levels)
erosion or ulceration (4 levels)

The 4 components are classified from 0 to 3, yielding a final score that ranges between 0 and 33 (Mosli et al., Gut; 66 (1): 50-58 (2017)).

### Geboes Score (Ulcerative Colitis)

Geboes score is a scoring system for microscopic disease activity that incorporates a number of histological items classified in 5 grades (Mosli et al., Inflamm Bowel Dis; 20 (3): 564-75 (2014)).

### Global Histologic Activity Score (Crohn's Disease)

The Global Histologic Activity Score consists of 8 items assessing acute and chronic inflammatory changes, epithelial damage, and the extent of inflammation (ie, the proportion of biopsy specimens affected). Each of the 8 items is scored, with the totals subsequently added together (D'Haens et al., Gastroenterology; 114 (2): 262-7 (1998)).

### Crohn's Disease Activity Index

The CDAI (Yoshida E M. Can J Gastroenterol; 13 (1): 65-73 (1999)) consists of 8 factors added up after adjustment with a weighting factor:
Number of liquid or soft stools each day for 7 days×2
Abdominal pain (graded from 0 to 3 on severity) each day for 7 days×5
General well-being, subjectively assessed from 0 (well) to 4 (terrible) each day for 7 days×7
Presence of complications×20
Taking Lomotil, Imodium, or opiates for diarrhea×30
Presence of an abdominal mass (0 as none, 2 as questionable, 5 as definite)×10
Hematocrit of <0.47 in men and <0.42 in women×6
Percentage deviation from standard weight×1

### Two-Item Patient-Reported Outcome (Ulcerative Colitis and Crohn's Disease)

UC: The 2-item patient-reported outcome (PRO2) includes daily evaluation of the 2 subjected items of the Mayo score: stool frequency and rectal bleeding. Each parameter of the score ranges from 0 (normal or inactive disease) to 3 (severe activity) and the total score from 0 to 6, respectively (Dragasevic et al., Gastroenterol Res Pract; 2020:2065383 (2020); Jairath et al., Aliment Pharmacol Ther; 42 (10): 1200-10 (2015)).

CD: PRO2 is the sum of the daily stool frequency (0 to 3) and abdominal pain (0 to 3) from the CDAI (Khanna et al., Aliment Pharmacol Ther; 41(1): 77-86 (2015)).

### Permitted Inflammatory Bowel Disease Medications and Rescue Medications

Permitted concomitant treatment for UC and CD must remain at a stable dose (no increase or decrease) during the study treatment period (except for decreases due to adverse events); if doses are increased, patients must be withdrawn from the study. The dose of any background medication for UC and CD should not be changed during the screening and double-blind treatment period.

Patients will be allowed to use the following medications as detailed below:
Concomitant use of oral 5-ASA or sulfasalazine. Dose must be stable for at least 4 weeks prior to endoscopy and through week 14. If oral 5-ASA treatment has been recently discontinued, it must have been stopped for at least 2 weeks prior to endoscopy.
A stable dose of oral corticosteroids (prednisone equivalent of up to 20 mg/day; budesonide of up to 9 mg/day) for at least 2 weeks prior to endoscopy and through week 14. If oral corticosteroids have been recently discontinued, they must have been stopped at least 2 weeks prior to endoscopy. Decreases in steroid use due to adverse events are allowed.
A stable dose of immunosuppressant drugs (methotrexate≤ (less than or equal to) 25 mg intramuscular or SC once weekly or ≤ (less than or equal to) 15 mg per os once weekly; 6-MP≤ (less than or equal to) 1.5 mg/kg/day; or AZA≤ (less than or equal to) 2.5 mg/kg/day) for 4 weeks prior to endoscopy and through week 14. Decreases due to adverse events are permitted.

### Safety and Tolerability Assessments

Safety is assessed throughout the study by evaluating reported adverse events, clinical laboratory test results, vital signs measurements, electrocardiogram (ECG) findings, physical examination findings, and use of concomitant medication. Local tolerability assessments are performed after each administration of TEV-48574/placebo and include administration site findings (e.g., erythema, ecchymosis, induration, tenderness, warmth, and swelling) and pain. Blood samples for anti-drug antibody (ADA) testing are collected throughout the study to show that the TEV-48574 is not immunogenic. Adverse events, administration site findings, and treatment-emergent ADA responses are documented throughout the study to show that the TEV-48574 is safe and tolerable.

### Efficacy and Safety Analysis

The results are analyzed to demonstrate the the efficacy and dose response of 3 different dose regimens of TEV-48574 subcutaneous (sc) administered every 2 weeks (Q2W) in adult patients with IBD (moderate to severe UC or CD) as assessed by induction of clinical remission (UC) and endoscopic response (CD) at week 14. Efficacy is demonstrated based on a response (yes or no) at week 14, where the response in UC patients is defined as clinical remission and response in CD patients is defined as endoscopic response.

As used herein, the term “clinical remission” of ulcerative colitis refers to the clinical remission determined as follows:
Clinical remission is a modified (9-point rectal bleeding, stool frequency, and endoscopy) Mayo score of ≤ (less than or equal to) 2 points, which is defined by: stool frequency subscore of 0 or 1, rectal bleeding subscore of 0, and endoscopic subscore of 0 or 1, where a score of 1 does not include “friability”

As used herein, the term “endoscopic response” for Crohn's disease refers to an endoscopic response determined as follows:
Endoscopic response is defined by as a reduction from baseline in Simple Endoscopic Score for Crohn's Disease (SES-CD) of at least 50%.

The following endpoints are also measured to demonstrate efficacy in patients with moderate to severe UC:
Clinical response at week 14, defined as a decrease from baseline in the modified (9-point rectal bleeding, stool frequency, and endoscopy) Mayo score of at least 2 points AND at least a 30% reduction from baseline with either a decrease in rectal bleeding subscore of at least 1 or an absolute rectal bleeding subscore of less than or equal to 1
Endoscopic improvement defined as a Mayo endoscopic subscore of 0 or 1 at week 14
Endoscopic remission defined as a Mayo endoscopic subscore of 0 at week 14
Clinical response defined as decrease from baseline of at least 50% in 2-item patient-reported outcome (PRO2; rectal bleeding and stool frequency) at weeks 2, 4, 6, 8, 10, 12, and 14
Clinical remission defined as score of rectal bleeding=0 and stool frequency=0 on the PRO2 scale at weeks 2, 4, 6, 8, 10, 12, and 14
Histological remission defined as a Robarts Histopathology Index of ≤ (less than or equal to) 5 at week 14
Histological remission defined as Geboes index score ≤ (less than or equal to) 3.1 at week 14

The following endpoints are also measured to demonstrate efficacy in patients with moderate to severe CD:
Clinical response defined as a ≥ (more than or equal to) 100-point decrease in Crohn's Disease Activity Index (CDAI) score from baseline at weeks 2, 4, 6, 8, 10, 12, and 14
Clinical remission defined as a CDAI score <150 at weeks 2, 4, 6, 8, 10, 12, and 14
Endoscopic remission defined as SES CD score of 0-2, or SES CD score of 0-4, with no individual sub score >1 at week 14
Clinical response defined as a decrease from baseline of at least 50% in PRO2 (PRO2 is defined as having 2 components, abdominal pain and stool frequency) at weeks 2, 4, 6, 8, 10, 12, and 14
Clinical remission defined as abdominal pain≤ (less than or equal to) 1 and stool frequency≤ (less than or equal to) 3 on the PRO2 scale at weeks 2, 4, 6, 8, 10, 12 and 14
Endoscopic response defined as a decrease in modified multiplier (MM)-SES-CD of >50% from baseline at week 14
Histologic response defined as a ≥ (more than or equal to) 50% decrease in Global Histologic Activity Score from baseline at week 14

The following measures/parameters are analyzed to demonstrate safety and tolerability:
Adverse events
Change from baseline in clinical laboratory test results (serum chemistry, hematology, and urinalysis)
Change from baseline in vital signs measurements (blood pressure, pulse rate, body temperature, and respiratory rate)
Change from baseline in 12-lead electrocardiogram (ECG) findings
Use of concomitant medication
Patients who stopped the TEV-48574/placebo due to adverse events
Local tolerability at the injection site
Device-related adverse events and malfunctions (for the commercial SC infusion system)

The following endpoints are analyzed to demonstrate a lack of immunogenicity:
Change from baseline in treatment-emergent anti-drug antibody (ADA) at weeks 2, 4, 8, 14, and follow-up visit
Presence of neutralizing ADA in ADA positive patients at weeks 2, 4, 8, 14, and follow-up visit.

The efficacy and safety results are analyzed to demonstrate that the efficacy and safety of the TEV-48574 dosing regimen.

### Example 2: Stability of Reconstituted TEV-48574 Drug Product Solution

The in-use stability of TEV-48574 reconstituted drug product was evaluated.

For this study, TEV-48574 was presented in lyophilized form at a protein concentration of 150 mg/mL in 10 mM Histidine, 5% (w/v) Sucrose, 100 mM arginine-hydrochloride (Arg-HCl), 0.02% (w/v) polysorbate-80 (PS-80) at pH 6.0. The lyophilized formulation was contained in 5 cc vials with 20 mm neck size. Vials were stored at 2-8° C. and brought to room temperature prior to reconstitution. Approximately 7 vials were reconstituted using 2.0 mL of sterile water for injection (WFI) in each vial to make stock solutions. The stock solutions were then diluted into different falcon tubes to concentrations of 50, 20 and 5 mg/mL using a formulation buffer of 10 mM Histidine, 5% (w/v) Sucrose, 100 mM Arg-HCl, and 0.02% (w/v) PS80, at pH 6.0. After dilution, 4 mL samples were subjected to incubation for 24 hours, 72 hours, and 10 days at 2-8° C. protected from light, and for 24 hours at room temperature under normal light conditions. Table 3 shows the specific time points and conditions tested.

TABLE 3
Conditions and Time Points Tested for
Reconstituted TEV-48574 Stability Study

| TEV-48574 | | | | 24 | 72 | 10 |
|---|---|---|---|---|---|---|
| concentrations | | Conditions | T0 | hours | hours | days |
| 50 | mg/mL | 2-8° C. (protected from light) | X | X | X | X |
| | | Room temperature (Normal | | X | | |
| | | Lighting conditions) | | | | |
| | | Sample volume required (mL) | 4 | 8 | 4 | 4 |
| 20 | mg/mL | 2-8° C. (protected from light) | X | X | X | X |
| | | Room temperature (Normal | | X | | |
| | | Lighting conditions) | | | | |
| | | Sample volume required (mL) | 4 | 8 | 4 | 4 |
| 5 | mg/mL | 2-8° C. (protected from light) | X | X | X | X |
| | | Room temperature (Normal | | X | | |
| | | Lighting conditions) | | | | |
| | | Sample volume required (mL) | 4 | 8 | 4 | 4 |

Note:
T0 = time zero

At the end of each time point and condition, samples were analyzed by visual appearance, pH, osmolality, protein concentration, size exclusion chromatography (SEC), capillary sodium dodecyl sulfate gel electrophoresis (cSDS), capillary isoelectric focusing (cIEF), and sub-visible particle analysis using micro flow imaging (MFI).

As shown in Table 4, no significant difference was seen in reconstituted samples upon dilution with respect to visual appearance, pH, osmolality and protein concentration. Also, pH and osmolality values of the samples were within the limits of 6.0±0.5 pH and 200-500 mOsm/kg, respectively.

TABLE 4
Visual Appearance, pH, Osmolality and Protein
Concentration of Reconstituted Product

| | | | Measured | | |
|---|---|---|---|---|---|
| | | | Protein | | |
| Condition and | | Osmolality | concentration | | |
| time point | pH | (mOsm/kg) | (mg/mL) | | Appearance |
| 5 mg/mL, T0 | 6.36 | 362 | 5.8 | mg/mL | Liquid, |
| | | | | | C, L, F |
| 5 mg/mL, RT, | 6.35 | 360 | 5.7 | mg/mL | Liquid, |
| 24 hours | | | | | C, L, F |
| 5 mg/mL, 2-8° C., | 6.34 | 358 | 4.5 | mg/mL | Liquid, |
| 24 hours | | | | | C, L, F |
| 5 mg/mL, 2-8° C., | 6.34 | 358 | 5.6 | mg/mL | Liquid, |
| 72 hours | | | | | C, L, F |
| 5 mg/mL, 2-8° C., | 6.36 | 364 | 5.7 | mg/mL | Liquid, |
| 10 days | | | | | C, L, F |
| 20 mg/mL, T0 | 6.38 | 370 | 22.0 | mg/mL | Liquid, |
| | | | | | C, L, F |
| 20 mg/mL, RT, | 6.34 | 363 | 22.2 | mg/mL | Liquid, |
| 24 hours | | | | | C, L, F |
| 20 mg/mL, 2-8° C., | 6.36 | 364 | 21.9 | mg/mL | Liquid, |
| 24 hours | | | | | C, L, F |
| 20 mg/mL, 2-8° C., | 6.35 | 363 | 22.3 | mg/mL | Liquid, |
| 72 hours | | | | | C, L, F |
| 20 mg/mL, 2-8° C., | 6.39 | 368 | 22.2 | mg/mL | Liquid, |
| 10 days | | | | | C, L, F |
| 50 mg/mL, T0 | 6.37 | 374 | 54.4 | mg/mL | Liquid, |
| | | | | | C, L, F |
| 50 mg/mL, RT, | 6.36 | 375 | 54.3 | mg/mL | Liquid, |
| 24 hours | | | | | C, L, F |
| 50 mg/mL, 2-8° C., | 6.37 | 372 | 55.2 | mg/mL | Liquid, |
| 24 hours | | | | | C, L, F |
| 50 mg/mL, 2-8° C., | 6.35 | 377 | 55.6 | mg/mL | Liquid, |
| 72 hours | | | | | C, L, F |
| 50 mg/mL, 2-8° C., | 6.36 | 375 | 54.8 | mg/mL | Liquid, |
| 10 days | | | | | C, L, F |

Note:
T0 = time zero;
RT = room temperature;
C = clear solution,
L = colorless,
F = free from visible particles

SEC analysis showed that the % monomer level of antibody in the samples did not have any significant change over the duration of the stability evaluation. See Table 5. These results indicate that storage of the reconstituted drug product upon dilution at 2-8° C. for up to 10 days did not have any effect on the % monomer level upon incubation.

TABLE 5
Percent Monomer Level of Reconstituted Product

| | | | % Main |
|---|---|---|---|
| Condition and time point | % HMW | % Dimer | peak |
| 5 mg/mL, T0 | 0.7 | 0.1 | 99.3 |
| 5 mg/mL, RT, 24 hours | 0.7 | 0.1 | 99.3 |
| 5 mg/mL, 2-8° C., 24 hours | 0.6 | 0.1 | 99.3 |
| 5 mg/mL, 2-8° C., 72 hours | 0.7 | 0.1 | 99.3 |
| 5 mg/mL, 2-8° C., 10 days | 0.6 | 0.1 | 99.3 |
| 20 mg/mL, T0 | 0.7 | 0.1 | 99.3 |
| 20 mg/mL, RT, 24 hours | 0.6 | 0.1 | 99.3 |
| 20 mg/mL, 2-8° C., 24 hours | 0.7 | 0.1 | 99.2 |
| 20 mg/mL, 2-8° C., 72 hours | 0.7 | 0.1 | 99.3 |
| 20 mg/mL, 2-8° C., 10 days | 0.7 | 0.1 | 99.2 |
| 50 mg/mL, T0 | 0.7 | 0.1 | 99.3 |
| 50 mg/mL, RT, 24 hours | 0.7 | 0.1 | 99.2 |
| 50 mg/mL, 2-8° C., 24 hours | 0.7 | 0.1 | 99.2 |
| 50 mg/mL, 2-8° C., 72 hours | 0.7 | 0.1 | 99.2 |
| 50 mg/mL, 2-8° C., 10 days | 0.7 | 0.1 | 99.2 |

Note:
T0 = time zero;
RT = room temperature;
HMW = high molecular weight species

cIEF analysis showed that dilution of reconstituted drug product and its incubation at 2-8° C. for up to 10 days does not have any significant impact on the charge heterogeneity profile of TEV-48574. See Table 6.

TABLE 6
cIEF Analysis of Reconstituted Product

| | % Acidic | % Main | % Basic |
|---|---|---|---|
| Condition and time point | species | peak | species |
| 5 mg/mL, T0 | 22.8 | 72.3 | 4.9 |
| 5 mg/mL, RT, 24 hours | 23.1 | 71.8 | 5.1 |
| 5 mg/mL, 2-8° C., 24 hours | 23.1 | 71.9 | 4.9 |
| 5 mg/mL, 2-8° C., 72 hours | 24.2 | 70.1 | 4.7 |
| 5 mg/mL, 2-8° C., 10 days | 23.0 | 72.6 | 4.4 |
| 20 mg/mL, T0 | 20.2 | 75.3 | 4.5 |
| 20 mg/mL, RT, 24 hours | 19.7 | 76.0 | 4.3 |
| 20 mg/mL, 2-8° C., 24 hours | 19.9 | 75.1 | 5.1 |
| 20 mg/mL, 2-8° C., 72 hours | 20.2 | 75.3 | 4.4 |
| 20 mg/mL, 2-8° C., 10 days | 19.6 | 75.8 | 4.5 |
| 50 mg/mL, T0 | 20.1 | 75.0 | 4.9 |
| 50 mg/mL, RT, 24 hours | 19.5 | 76.2 | 4.2 |
| 50 mg/mL, 2-8° C., 24 hours | 19.8 | 75.7 | 4.5 |
| 50 mg/mL, 2-8° C., 72 hours | 19.7 | 76.1 | 4.3 |
| 50 mg/mL, 2-8° C., 10 days | 19.0 | 76.8 | 4.2 |

Note:
T0 = time zero

As shown in Table 7, cSDS analysis revealed that reconstituted drug product purity was >90%, and that dilution and incubation of reconstituted drug product at different conditions for various time points had no significant impact on purity.

TABLE 7
cSDS Analysis of Reconstituted Product

| | NR-cSDS | | | R-cSDS |
|---|---|---|---|---|
| | | % | | % Reduced |
| Condition and | % | Main | % | purity (% |
| time point | fragment | peak | HMW | HC + % LC) |
| 5 mg/mL, T0 | 1.7 | 98.3 | 0.0 | 96.3 |
| 5 mg/mL, RT, | 0.0 | 100.0 | 0.0 | 100.0 |
| 24 hours | | | | |
| 5 mg/mL, 2-8° C., | 1.8 | 98.2 | 0.0 | 95.1 |
| 24 hours | | | | |
| 5 mg/mL, 2-8° C., | 0.0 | 100.0 | 0.0 | 100.0 |
| 72 hours | | | | |
| 5 mg/mL, 2-8° C., | 0.0 | 100.0 | 0.0 | 100.0 |
| 10 days | | | | |
| 20 mg/mL, T0 | 1.6 | 94.5 | 3.9 | 94.1 |
| 20 mg/mL, RT, | 1.9 | 95.2 | 2.9 | 93.2 |
| 24 hours | | | | |
| 20 mg/mL, 2-8° C., | 3.6 | 94.2 | 2.2 | 93.0 |
| 24 hours | | | | |
| 20 mg/mL, 2-8° C., | 1.8 | 98.2 | 0.0 | 93.5 |
| 72 hours | | | | |
| 20 mg/mL, 2-8° C., | 1.9 | 96.8 | 1.3 | 93.5 |
| 10 days | | | | |
| 50 mg/mL, T0 | 3.8 | 92.6 | 3.7 | 92.9 |
| 50 mg/mL, RT, | 1.6 | 95.1 | 3.4 | 93.3 |
| 24 hours | | | | |
| 50 mg/mL, 2-8° C., | 1.5 | 96.0 | 2.5 | 92.6 |
| 24 hours | | | | |
| 50 mg/mL, 2-8° C., | 3.4 | 93.9 | 2.7 | 93.5 |
| 72 hours | | | | |
| 50 mg/mL, 2-8° C., | 2.1 | 96.4 | 1.5 | 93.5 |
| 10 days | | | | |

Note:
NR = nonreducing;
R = reducing;
HC = heavy chain;
LC = light chain;
HMW = high molecular weight species;
T0 = time zero;
RT = room temperature

MFI analysis showed that 50 mg/mL samples contained a slightly higher number of sub-visible particles. See Table 8. However, no significant increase in particle concentration was observed overall for different concentration samples when comparing initial time point samples to later time point samples.

TABLE 8
Particle Size Concentration of Reconstituted Product

| | Particle concentration (#/mL) | | | |
|---|---|---|---|---|
| Condition and time point | ≥2 um | ≥5 um | ≥10 um | ≥25 um |
| 5 mg/mL, T0 | 1164 | 306 | 76 | 4 |
| 5 mg/mL, RT, 24 hours | 843 | 197 | 45 | 5 |
| 5 mg/mL, 2-8° C., 24 hours | 296 | 46 | 10 | 3 |
| 5 mg/mL, 2-8° C., 72 hours | 1132 | 317 | 80 | 9 |
| 5 mg/mL, 2-8° C., 10 days | 4981 | 1322 | 253 | 10 |
| 20 mg/mL, T0 | 869 | 95 | 18 | 1 |
| 20 mg/mL, RT, 24 hours | 814 | 72 | 5 | 1 |
| 20 mg/mL, 2-8° C., 24 hours | 808 | 84 | 8 | 0 |
| 20 mg/mL, 2-8° C., 72 hours | 1199 | 197 | 40 | 7 |
| 20 mg/mL, 2-8° C., 10 days | 1537 | 289 | 60 | 7 |
| 50 mg/mL, T0 | 1706 | 178 | 21 | 4 |
| 50 mg/mL, RT, 24 hours | 1459 | 152 | 25 | 2 |
| 50 mg/mL, 2-8° C., 24 hours | 2812 | 568 | 124 | 9 |
| 50 mg/mL, 2-8° C., 72 hours | 3240 | 682 | 159 | 18 |
| 50 mg/mL, 2-8° C., 10 days | 2022 | 356 | 56 | 6 |

Note:
T0 = time zero;
RT = room temperature

Taken together, these results show that reconstituted TEV-48574 drug product is stable upon dilution over a period of 10 days upon storage at 2-8° C. protected from light and for 24 hours at room temperature under normal light conditions.

### Example 3: Stability of TEV-48574 Liquid Formulations at 100 mg/ml and 150 mg/ml

The purpose of this study was to evaluate the long term stability of liquid and lyophilized TEV-48574 formulations. Three formulations were tested: a lyophilized form containing 100 mg/mL of drug product in 10 mM Histidine, 5% (w/v) Sucrose, 100 mM Arg-HCl, 0.02% (w/v) PS-80 at pH 6.0 (Formulation 1), and two liquid forms containing 100 mg/mL or 150 mg/mL of drug product in 10 mM Histidine, 5% (w/v) Sucrose, 100 mM Arg-HCl, 0.02% (w/v) PS-80 at pH 6.0 (Formulations 2 and 3, respectively). The stability of these formulations was evaluated under the following conditions in 5 cc vials (Type I glass): “standard” condition (2-8° C.), “accelerated” condition (25±2° C./60±5% relative humidity (RH)), and “stressed” condition (40±2° C./75±5% RH). Furthermore, the stability of Formulation 3 was evaluated under the same conditions but with the formulation filled into 2.25 mL Nipro pre-filled syringes (Nipro PFS) fitted with West plunger-stoppers. The impact of these conditions on several product quality attributes was tested for the formulations.

### Visual Appearance, Protein Concentration, Osmolality and Viscosity

The results of a visual appearance, protein concentration and osmolality analysis for Formulations 1-3 are shown in Tables 9-12. At standard storage conditions of 2-8° C., no significant difference across the formulations was observed. At intermittent time points, few visible particles were observed. However, this could be related to the development nature of the study where drug product was manually filled into vials. Furthermore, these particles cannot be product-related as they did not increase over time and were not detected consistently in all the samples across different time points. For visual appearance at stressed conditions of 40° C., the solutions appeared slightly yellow at later time points. No significant differences were observed between Formulation 3 filled into Nipro PFS compared to Formulation 3 stored in glass vials up to 24 months with respect to visual appearance, protein concentration, osmolality, and viscocity (data not shown), indicating that these attributes of the formulation are not affected by contact with the Nipro PFS.

The measured protein concentration for all three formulations at standard (2-8° C.), accelerated (25° C.), and stressed (40° C.) conditions were close to the nominal concentration expected and did not vary over time. Similar trends were observed for osmolality and viscosity measured at standard storage conditions at time zero (T0), 24 months (24M), and 36 months (36M) with no significant difference observed over time.

TABLE 9
Visual Appearance and Protein Concentration Results at 2-8° C.

| | | | | Protein concentra- | | |
|---|---|---|---|---|---|---|
| Time | Visual appearance | | | tion (mg/mL) | | |
| Point | F1 | F2 | F3 | F1 | F2 | F3 |
| T0 | L; S; FFVP | L; S; 1-3 | L; S; 1 | 103.1 | 106.2 | 153.4 |
| | | particle | particle | | | |
| | | observed | observed | | | |
| 1 M | L; S; FFVP | L; S; 1 | L; S; FFVP | 99.7 | 106.8 | 154.2 |
| | | particle | | | | |
| | | observed | | | | |
| 3 M | L; S; FFVP | L; S; FFVP | L; S; FFVP | 99.5 | 107.1 | 155.7 |
| 6 M | L; S; FFVP | L; S; 1 Fibrous | L; S; FFVP | 99.3 | 105.5 | 148.8 |
| | | particle | | | | |
| | | detected | | | | |
| 9 M | L; S; FFVP | L; S; FFVP | L; S; FFVP | 93.4 | 105.2 | 152.1 |
| 12 M | NT | L; S; FFVP | L; S; FFVP | 102.2 | 106.9 | 149.6 |
| 18 M | L; S; FFVP | L; S; 1 | L; S; FFVP | 94.4 | 102.9 | 153.1 |
| | | particle | | | | |
| | | observed | | | | |
| 24 M | L; S; FFVP | L; S; FFVP | S; SY; FFVP | 97.4 | 104.9 | 148.8 |
| 36 M | L; S; FFVP | L; S; FFVP | L; S; FFVP | 101.1 | 106.3 | 153.2 |

T0: Time zero;
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

TABLE 10
Osmolality and Viscosity Results at 2-8° C.

| Time | Osmolality (mOsm/kg) | | | Viscosity (cP) measured at 20° C. | | |
|---|---|---|---|---|---|---|
| Point | F1 | F2 | F3 | F1 | F2 | F3 |
| T0 | 379 | 390 | 434 | 3.4 | 3.5 | 7.8 |
| 24 M | 360 | 379 | 425 | 3.3 | 3.5 | 7.6 |
| 36 M | 401 | 399 | 462 | NT | NT | NT |

T0: Time zero;
M: Month(s);
F1: Formulation 1;
F2: Formulation 2;
F3: Formulation 3;
NT: Not tested

TABLE 11
Visual Appearance and Protein Concentration Results at 25° C.

| Time | Visual appearance | | | Protein concentration (mg/mL) | | |
|---|---|---|---|---|---|---|
| point | F1 | F2 | F3 | F1 | F2 | F3 |
| T0 | L; S; FFVP | L; S; 1-3 | L; S; 1 | 103.1 | 106.2 | 153.4 |
| | | particles | particle | | | |
| | | observed | observed | | | |
| 2 WK | L; S; FFVP | L; S; FFVP | L; S; 1-2 | 100.8 | 106.7 | 153.9 |
| | | | particles | | | |
| 1 M | L; S; FFVP | L; S; 1 | L; S; FFVP | 101.5 | 107.2 | 155 |
| | | particle | | | | |
| | | observed | | | | |
| 2 M | L; S; FFVP | L; S; FFVP | L; S; FFVP | 101.2 | 106.8 | 153.6 |
| 3 M | L; S; Few | L; S; FFVP | L; S; FFVP | 99 | 106.2 | 153.5 |
| | Particles | | | | | |
| 6 M | L; S; FFVP | L; S; FFVP | L; S; FFVP | 97.7 | 106.8 | 151.7 |
| 9 M | L; S; FFVP | L; S; FFVP | L; S; 1 | 97.4 | 106.4 | 156.8 |
| | | | particle | | | |
| | | | observed | | | |
| 12 M | NT | L; S; 1 | L; S; 1-2 | 101 | 107.7 | 153 |
| | | particle | particles | | | |
| | | observed | observed | | | |

T0: Time zero;
WK: Week(s);
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

TABLE 12
Visual Appearance and Protein Concentration Results at 40° C.

| Time | Visual appearance | | | Protein concentration (mg/mL) | | |
|---|---|---|---|---|---|---|
| point | F1 | F2 | F3 | F1 | F2 | F3 |
| T0 | L; S; FFVP | L; S; 1-3 | L; S; 1 | 103.1 | 106.2 | 153.4 |
| | | particle | particle | | | |
| | | observed | observed | | | |
| 2 WK | L; S; FFVP | L; S; 1 | L; S; FFVP | 100.5 | 105.8 | 153.1 |
| | | particle | | | | |
| | | observed | | | | |
| 1 M | L; S; 1 | L; S; 1 | L; S; FFVP | 99.9 | 106.7 | 154.9 |
| | particle | particle | | | | |
| | observed | observed | | | | |
| 2 M | L; S; FFVP | L; S; FFVP | S; SY; FFVP | 100.9 | 106.7 | 152.5 |
| 3 M | L; S; Few | L; S; FFVP | S; SY; Few | 99.1 | 106.7 | 154.5 |
| | particles | | particles | | | |
| | | | were | | | |
| | | | observed | | | |
| 6 M | L; S; FFVP | S; SY; FFVP | S; SY; FFVP | 95.4 | 106.1 | 153.0 |

T0: Time zero;
WK: Week(s);
M: Month(s);
F1: Formulation 1;
F2: Formulation 2;
F3: Formulation 3;
S: Slightly opalescent;
L: Colorless;
FFVP: Free from visible particles;
SY: Slightly yellow;
NT: Not tested

### Size Exclusion Chromatography (SEC)

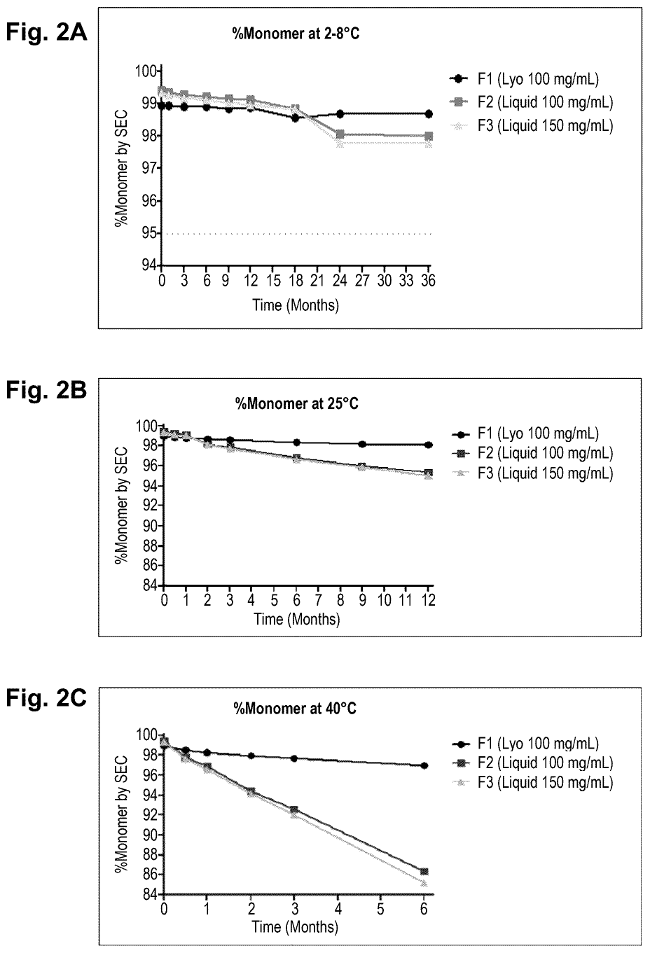

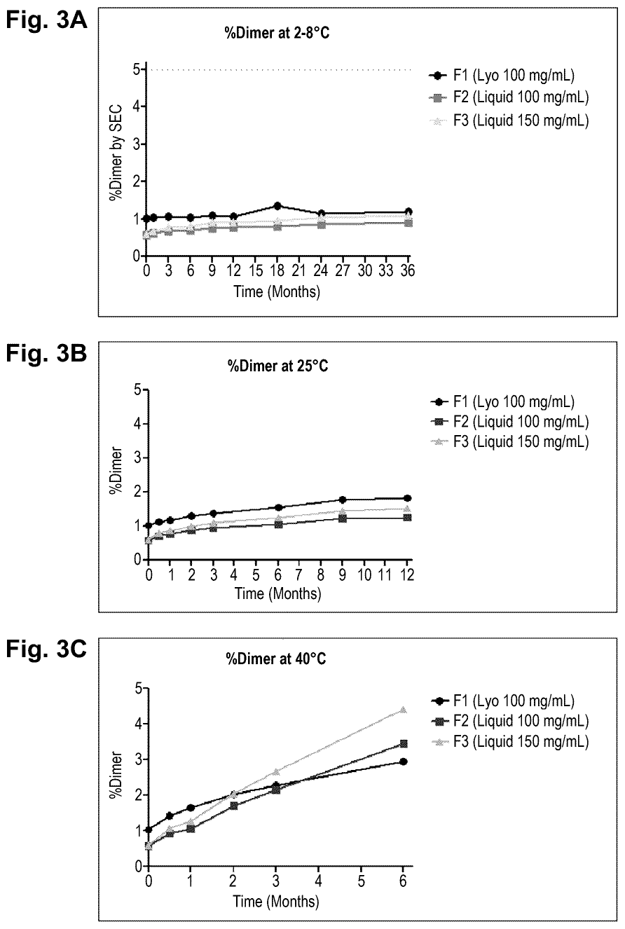

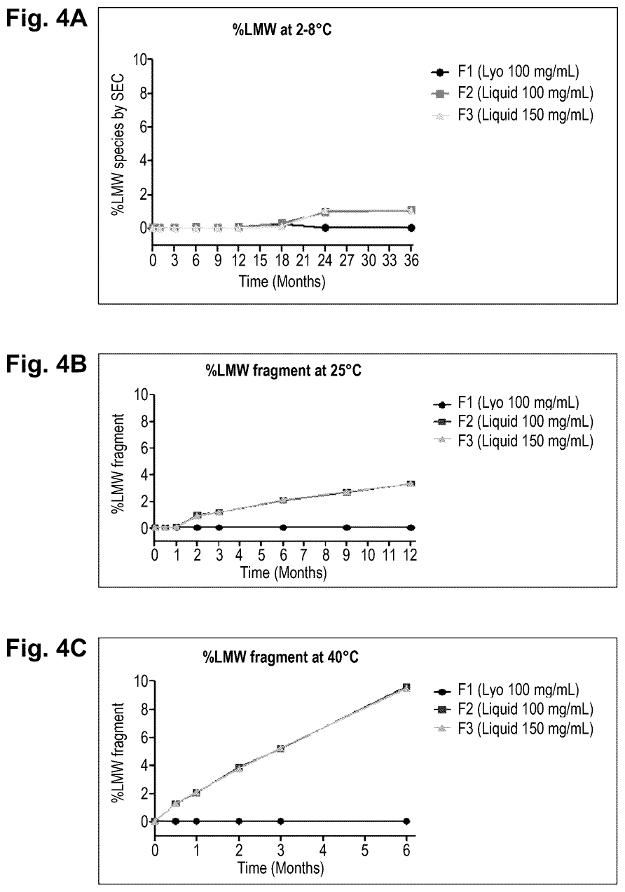

Tables 13-15 and FIGS. 2A-2C, 3A-3C and 4A-4C show the percent (%) monomer, % dimer and % low molecular weight species of Formulations 1, 2 and 3 measured by SEC. All three formulations stored at standard storage conditions of 2-8° C. met the acceptance criteria of % monomer and % dimer levels for up to 24 months and for up to 36 months. A slight decrease in % monomer with concurrent increase in % low molecular weight species was observed for Formulations 2 and 3 compared to Formulation 1. However, this is an expected observation considering that lyophilized formulations are typically more stable compared to liquid formulations. At accelerated and stressed conditions, protein fragmentation was more prevalent, indicating formation of low molecular weight species as the primary degradation pathway for liquid formulations. The rates of protein degradation of Formulation 2 and Formulation 3 were similar, indicating no significant impact of protein concentration on degradation. No significant differences were observed between Formulation 3 filled into Nipro PFS compared to Formulation 3 stored in glass vials with respect to % monomer, % dimer, and % low molecular weight species up to 24 months (data not shown), indicating that the stability profile of the formulation is not affected by contact with Nipro PFS.

TABLE 13
% Monomer, % Dimer and % Low Molecular Weight Species at 2-8° C.

| | Formulation 1 | | | Formulation 2 | | | Formulation 3 | | |
|---|---|---|---|---|---|---|---|---|---|
| | % Monomer | % Dimer | | % Monomer | % Dimer | | % Monomer | % Dimer | |
| | (Acceptance | (Acceptance | | (Acceptance | (Acceptance | | (Acceptance | (Acceptance | |
| Time | crite- | crite- | % | crite- | crite- | % | crite- | crite- | % |
| (Months) | ria: ≥95.0%) | ria: ≤5.0%) | LMW | ria: ≥95.0%) | ria: ≤5.0%) | LMW | ria: ≥95.0%) | ria: ≤5.0%) | LMW |
| 0 | 98.93 | 1.03 | 0.04 | 99.39 | 0.57 | 0.04 | 99.35 | 0.60 | 0.04 |
| 1 | 98.92 | 1.04 | 0.04 | 99.34 | 0.62 | 0.04 | 99.27 | 0.68 | 0.05 |
| 3 | 98.90 | 1.06 | 0.04 | 99.26 | 0.68 | 0.05 | 99.17 | 0.78 | 0.06 |
| 6 | 98.90 | 1.05 | 0.05 | 99.20 | 0.70 | 0.09 | 99.11 | 0.81 | 0.08 |
| 9 | 98.85 | 1.09 | 0.04 | 99.16 | 0.77 | 0.07 | 99.02 | 0.91 | 0.07 |
| 12 | 98.86 | 1.08 | 0.03 | 99.12 | 0.78 | 0.09 | 98.98 | 0.92 | 0.08 |
| 18 | 98.56 | 1.35 | 0.29 | 98.84 | 0.80 | 0.33 | 98.84 | 0.97 | 0.16 |
| 24 | 98.70 | 1.14 | 0.04 | 98.07 | 0.85 | 1.00 | 97.80 | 1.04 | 1.11 |
| 36 | 98.70 | 1.19 | 0.04 | 98.00 | 0.91 | 1.10 | 97.80 | 1.09 | 1.10 |

LMW: Low molecular weight species

TABLE 14
% Monomer, % Dimer and % Low Molecular Weight Species at 25° C.

| | Formulation 1 | | | Formulation 2 | | | Formulation 3 | | |
|---|---|---|---|---|---|---|---|---|---|
| Time | % | % | % | % | % | % | % | % | % |
| (Months) | Monomer | Dimer | LMW | Monomer | Dimer | LMW | Monomer | Dimer | LMW |
| 0 | 98.93 | 1.03 | 0.04 | 99.39 | 0.57 | 0.04 | 99.35 | 0.60 | 0.04 |
| 0.5 | 98.83 | 1.13 | 0.04 | 99.21 | 0.71 | 0.08 | 99.11 | 0.80 | 0.08 |
| 1 | 98.77 | 1.19 | 0.04 | 99.11 | 0.78 | 0.11 | 99.00 | 0.89 | 0.11 |
| 2 | 98.65 | 1.31 | 0.04 | 98.12 | 0.89 | 0.99 | 98.08 | 1.00 | 0.92 |
| 3 | 98.56 | 1.39 | 0.05 | 97.84 | 0.95 | 1.21 | 97.68 | 1.10 | 1.22 |
| 6 | 98.39 | 1.55 | 0.06 | 96.84 | 1.06 | 2.10 | 96.62 | 1.26 | 2.12 |
| 9 | 98.18 | 1.77 | 0.04 | 96.04 | 1.23 | 2.72 | 95.81 | 1.46 | 2.73 |
| 12 | 98.08 | 1.83 | 0.03 | 95.36 | 1.26 | 3.33 | 95.06 | 1.52 | 3.36 |

LMW: Low molecular weight species

TABLE 15
% Monomer, % Dimer and % Low Molecular Weight at 40° C.

| | Formulation 1 | | | Formulation 2 | | | Formulation 3 | | |
|---|---|---|---|---|---|---|---|---|---|
| Time | % | % | % | % | % | % | % | % | % |
| (Months) | Monomer | Dimer | LMW | Monomer | Dimer | LMW | Monomer | Dimer | LMW |
| 0 | 98.93 | 1.03 | 0.04 | 99.39 | 0.57 | 0.04 | 99.35 | 0.60 | 0.04 |
| 0.5 | 98.54 | 1.42 | 0.04 | 97.75 | 0.92 | 1.33 | 97.59 | 1.07 | 1.34 |
| 1 | 98.30 | 1.65 | 0.04 | 96.87 | 1.05 | 2.08 | 96.58 | 1.28 | 2.13 |
| 2 | 97.92 | 2.04 | 0.04 | 94.41 | 1.70 | 3.89 | 94.13 | 2.06 | 3.80 |
| 3 | 97.67 | 2.29 | 0.04 | 92.61 | 2.16 | 5.23 | 92.06 | 2.68 | 5.26 |
| 6 | 96.99 | 2.95 | 0.06 | 86.36 | 3.45 | 9.60 | 85.26 | 4.42 | 9.54 |

LMW: Low molecular weight species

### Capillary Gel Electrophoresis (Reducing and Non-Reducing)

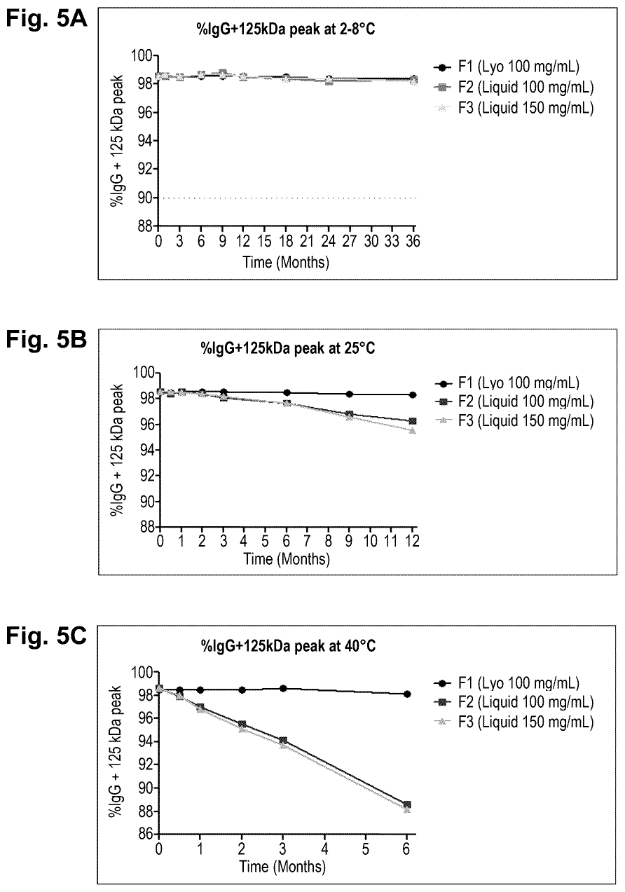

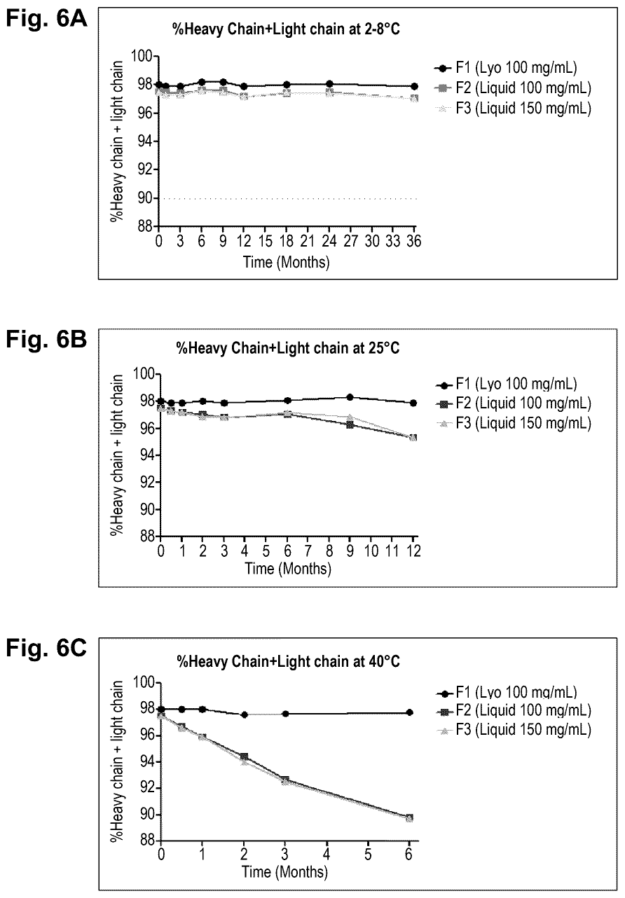

The percent (%) immunoglobulin G (IgG)+125 kDa peak was determined for Formulations 1-3 using non-reducing capillary gel electrophoresis (CGE). In addition, the % heavy chain+light chain was determined for Formulations 1-3 using reducing CGE. These results are shown in Tables 16-17 and FIGS. 5A-5C and 6A-6C, respectively. In all three formulations stored at standard storage conditions of 2-8° C., the % purity met acceptance criteria for up to 24 months and for up to 36 months. At accelerated and stressed conditions, protein fragmentation was observed for Formulations 2 and 3 compared to Formulation 1. However, this is an expected observation considering that lyophilized formulations are typically more stable compared to liquid formulations. In addition, the protein degradation trends for Formulations 2 and 3 were similar, indicating no significant impact of protein concentration. No significant differences were observed between Formulation 3 filled into Nipro PFS compared to Formulation 3 stored in glass vials with respect to the % IgG+125 kDa Peak up to 24 months (data not shown), indicating that the stability profile of the formulation is not affected by contact with Nipro PFS.

TABLE 16
% IgG + 125 kDa Peak Measured Using Non-Reducing CGE for Formulations
1, 2 and 3 at 2-8° C., 25° C. and 40° C.

| | F1 | F2 | F3 | | | | | | |
|---|---|---|---|---|---|---|---|---|---|
| Time | 2-8° C.; Acceptance | | | F1 | F2 | F3 | F1 | F2 | F3 |
| (Months) | criteria: ≥90.0% | | | 25° C. Conditions | | | 40° C. Conditions | | |
| 0 | 98.5 | 98.6 | 98.6 | 98.5 | 98.6 | 98.6 | 98.5 | 98.6 | 98.6 |
| 0.5 | NT | NT | NT | 98.5 | 98.4 | 98.6 | 98.5 | 97.9 | 98.0 |
| 1 | 98.6 | 98.6 | 98.7 | 98.6 | 98.6 | 98.5 | 98.5 | 97.0 | 96.8 |
| 2 | NT | NT | NT | 98.6 | 98.4 | 98.4 | 98.5 | 95.5 | 95.1 |
| 3 | 98.5 | 98.5 | 98.6 | 98.6 | 98.1 | 98.2 | 98.6 | 94.1 | 93.7 |
| 6 | 98.6 | 98.7 | 98.7 | 98.5 | 97.7 | 97.7 | 98.1 | 88.6 | 88.2 |
| 9 | 98.6 | 98.8 | 98.7 | 98.4 | 96.8 | 96.6 | NT | NT | NT |
| 12 | 98.6 | 98.5 | 98.6 | 98.3 | 96.3 | 95.6 | NT | NT | NT |
| 18 | 98.5 | 98.4 | 98.4 | NT | NT | NT | NT | NT | NT |
| 24 | 98.4 | 98.2 | 98.4 | NT | NT | NT | NT | NT | NT |
| 36 | 98.4 | 98.3 | 98.2 | NT | NT | NT | NT | NT | NT |

NT: Test not performed

TABLE 17
% Heavy Chain and Light Chain Measured Using Reducing CGE for Formulations
1, 2 and 3 at 2-8° C., 25° C. and 40° C.

| | F1 | F2 | F3 | | | | | | |
|---|---|---|---|---|---|---|---|---|---|
| Time | 2-8° C.; Acceptance | | | F1 | F2 | F3 | F1 | F2 | F3 |
| (Months) | criteria: ≥90.0% | | | 25° C. Conditions | | | 40° C. Conditions | | |
| 0 | 98.0 | 97.5 | 97.5 | 98.0 | 97.5 | 97.5 | 98.0 | 97.5 | 97.5 |
| 0.5 | NT | NT | NT | 97.9 | 97.3 | 97.3 | 98.0 | 96.7 | 96.6 |
| 1 | 97.9 | 97.5 | 97.3 | 97.9 | 97.2 | 97.2 | 98.0 | 95.9 | 95.9 |
| 2 | NT | NT | NT | 98.0 | 97.1 | 96.9 | 97.6 | 94.4 | 94.0 |
| 3 | 97.9 | 97.4 | 97.3 | 97.9 | 96.8 | 96.8 | 97.7 | 92.7 | 92.5 |
| 6 | 98.2 | 97.6 | 97.6 | 98.1 | 97.1 | 97.2 | 97.8 | 89.8 | 89.7 |
| 9 | 98.2 | 97.6 | 97.5 | 98.3 | 96.3 | 96.9 | NT | NT | NT |
| 12 | 97.9 | 97.2 | 97.2 | 97.9 | 95.3 | 95.3 | NT | NT | NT |
| 18 | 98.0 | 97.4 | 97.5 | NT | NT | NT | NT | NT | NT |
| 24 | 98.1 | 97.5 | 97.4 | NT | NT | NT | NT | NT | NT |
| 36 | 97.9 | 97.1 | 97.1 | NT | NT | NT | NT | NT | NT |

NT: Test not performed

Capillary Isoelectric Focusing (icIEF)

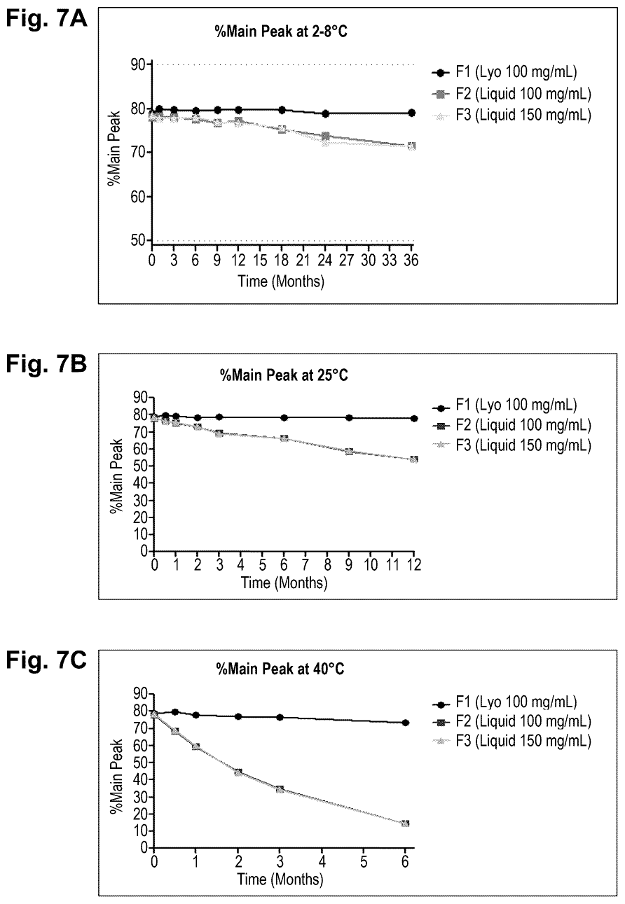

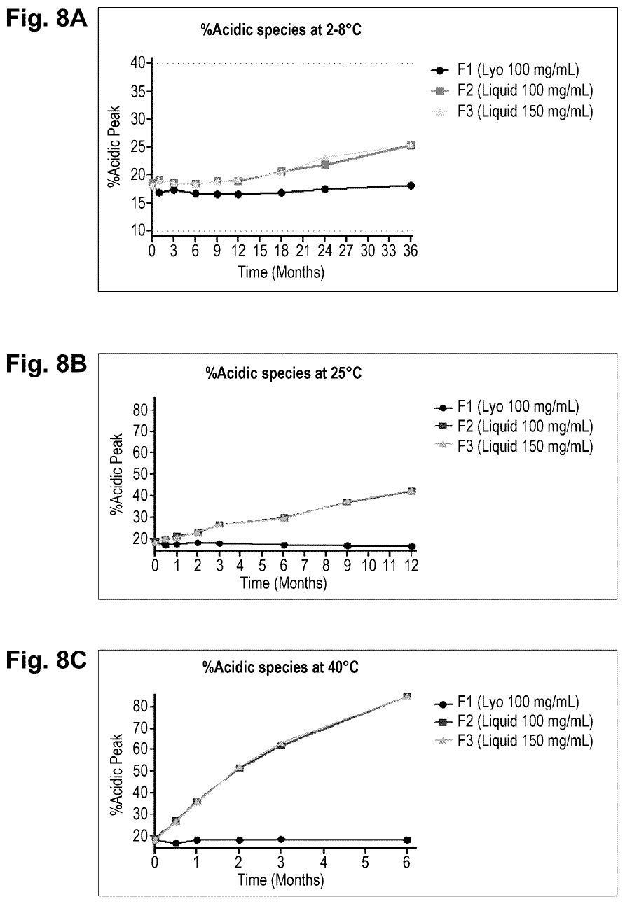

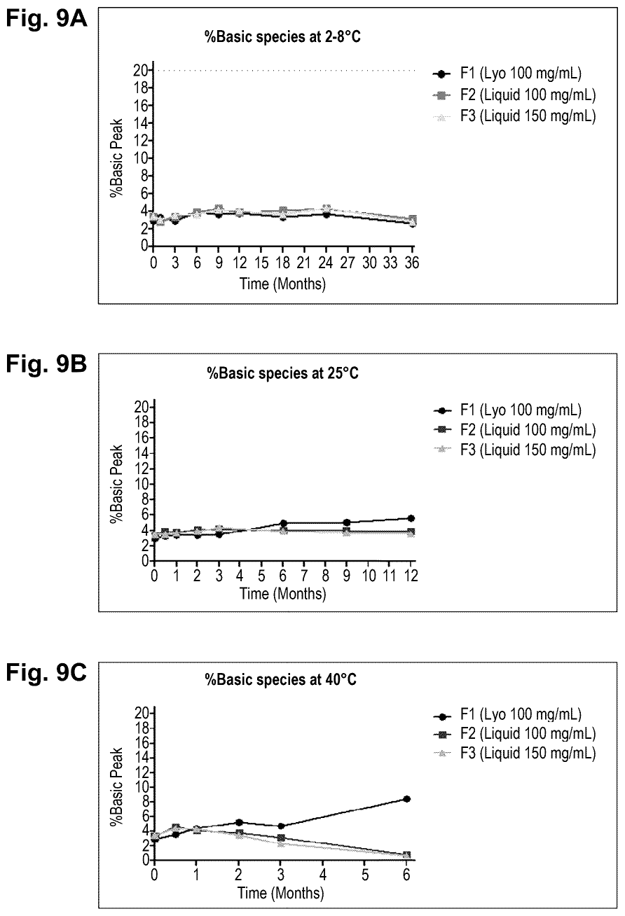

The percent (%) content of main species (main peak), % acidic species (acidic peak), and % basic species (basic peak) of Formulations 1-3 (F1-F3) were measured using capillary isoelectric focusing (icIEF). The results are shown in Tables 18-20. An overlay of stability trends in charge heterogeneity is presented in FIGS. 7A-7C, 8A-8C and 9A-9C. At standard storage conditions of 2-8° C. (FIG. 7A, 8A and 9A), all three formulations (F1-F3) met acceptance criteria for up to 24 months and for up to 36 months. At accelerated (25° C.; FIG. 7B, 8B and 9B) and stressed (40° C.; FIG. 7C, 8C and 9C) storage conditions, a decrease in % main peak with a concurrent increase in % acidic species was observed for Formulations 2 and 3. No significant change was observed for Formulation 1. However, this is an expected observation considering that lyophilized formulations are typically more stable compared to liquid formulations. No significant differences were observed between Formulation 3 filled into Nipro PFS compared to Formulation 3 stored in glass vials with respect to % main peak, % acidic species and % basic species up to 24 months (data not shown), indicating that the stability profile of the formulation is not affected by contact with Nipro PFS.

TABLE 18
% Main Peak, % Acidic Species and % Basic Species Determined Using icIEF for Formulations 1, 2 and 3 at 2-8° C.

| | Formulation 1 at 2-8° C. | | | Formulation 2 at 2-8° C. | | | Formulation 3 at 2-8° C. | | |
|---|---|---|---|---|---|---|---|---|---|
| | % Main | % Acidic | % Basic | % Main | % Acidic | % Basic | % Main | % Acidic | % Basic |
| | peak | species | species | peak | species | species | peak | species | species |
| | (Acceptance | (Acceptance | (Acceptance | (Acceptance | (Acceptance | (Acceptance | (Acceptance | (Acceptance | (Acceptance |
| Time | criteria: | criteria: | criteria: | criteria: | criteria: | criteria: | criteria: | criteria: | criteria: |
| (Months) | 50-90%) | 10-40%) | 0-20%) | 50-90%) | 10-40%) | 0-20%) | 50-90%) | 10-40%) | 0-20%) |
| 0.0 | 79.0 | 18.1 | 2.9 | 78.1 | 18.6 | 3.3 | 78.5 | 18.1 | 3.5 |
| 1.0 | 79.9 | 16.9 | 3.2 | 78.1 | 19.0 | 2.8 | 77.8 | 19.1 | 3.0 |
| 3.0 | 79.7 | 17.4 | 2.9 | 78.1 | 18.6 | 3.4 | 77.8 | 18.6 | 3.6 |
| 6.0 | 79.4 | 16.7 | 3.9 | 77.7 | 18.4 | 3.9 | 78.0 | 18.4 | 3.7 |
| 9.0 | 79.7 | 16.6 | 3.7 | 76.7 | 19.0 | 4.3 | 76.9 | 18.9 | 4.2 |
| 12.0 | 79.7 | 16.5 | 3.8 | 77.1 | 19.0 | 3.9 | 76.7 | 19.2 | 4.1 |
| 18.0 | 79.7 | 16.9 | 3.4 | 75.3 | 20.7 | 4.1 | 75.7 | 20.6 | 3.7 |
| 24.0 | 78.8 | 17.5 | 3.7 | 73.9 | 21.8 | 4.3 | 72.3 | 23.3 | 4.4 |
| 36.0 | 79.1 | 18.2 | 2.6 | 71.4 | 25.4 | 3.2 | 71.6 | 25.5 | 2.9 |

TABLE 19
% Main Peak, % Acidic Species and % Basic Species Determined
Using icIEF for Formulations 1, 2 and 3 at 25° C.

| | Formulation 1 at 25° C. | | | Formulation 2 at 25° C. | | | Formulation 3 at 25° C. | | |
|---|---|---|---|---|---|---|---|---|---|
| Time | % Main | % Acidic | % Basic | % Main | % Acidic | % Basic | % Main | % Acidic | % Basic |
| (Months) | peak | species | species | peak | species | species | peak | species | species |
| 0.0 | 79.0 | 18.1 | 2.9 | 78.1 | 18.6 | 3.3 | 78.5 | 18.1 | 3.5 |
| 0.5 | 79.7 | 17.0 | 3.3 | 76.8 | 19.3 | 3.9 | 76.2 | 20.3 | 3.5 |
| 1.0 | 79.2 | 17.5 | 3.3 | 75.1 | 21.1 | 3.8 | 75.7 | 20.6 | 3.7 |
| 2.0 | 78.6 | 18.0 | 3.4 | 73.1 | 22.8 | 4.1 | 73.1 | 23.0 | 3.9 |
| 3.0 | 78.9 | 17.6 | 3.5 | 69.2 | 26.6 | 4.2 | 69.0 | 26.5 | 4.5 |
| 6.0 | 78.3 | 16.8 | 4.9 | 66.1 | 29.9 | 4.0 | 66.4 | 29.7 | 3.9 |
| 9.0 | 78.4 | 16.5 | 5.1 | 58.8 | 37.2 | 4.0 | 58.9 | 37.4 | 3.7 |
| 12.0 | 78.1 | 16.3 | 5.6 | 54.1 | 42.0 | 3.9 | 53.9 | 42.5 | 3.6 |

TABLE 20
% Main Peak, % Acidic Species and % Basic Species Determined
Using icIEF for Formulations 1, 2 and 3 at 40° C.

| | Formulation 1 at 40° C. | | | Formulation 2 at 40° C. | | | Formulation 3 at 40° C. | | |
|---|---|---|---|---|---|---|---|---|---|
| Time | % Main | % Acidic | % Basic | % Main | % Acidic | % Basic | % Main | % Acidic | % Basic |
| (Months) | peak | species | species | peak | species | species | peak | species | species |
| 0.0 | 79.0 | 18.1 | 2.9 | 78.1 | 18.6 | 3.3 | 78.5 | 18.1 | 3.5 |
| 0.5 | 79.7 | 16.6 | 3.6 | 68.4 | 27.0 | 4.7 | 68.9 | 26.8 | 4.4 |
| 1.0 | 77.7 | 17.9 | 4.4 | 59.5 | 36.4 | 4.1 | 60.0 | 35.8 | 4.3 |
| 2.0 | 76.9 | 17.9 | 5.2 | 44.6 | 51.6 | 3.8 | 44.3 | 52.2 | 3.5 |
| 3.0 | 76.8 | 18.5 | 4.7 | 34.9 | 61.9 | 3.2 | 34.3 | 63.4 | 2.3 |
| 6.0 | 73.6 | 18.0 | 8.4 | 14.3 | 84.8 | 0.9 | 14.3 | 84.9 | 0.8 |

### Sub-visible Particles Using Micro-flow Imaging (MFI)

Sub-visible particles in Formulations 1-3 were measured at different time points at standard conditions of 2-8° C., accelerated conditions of 25° C., and stressed conditions of 40° C. The results are shown in Tables 21-23, respectively. No significant changes in sub-visible particles were observed. At intermittent time points, higher sub-visible particles were observed compared to other time points. This could be related to the method where greater variability and sensitivity have been observed for sub-visible particles measured using MFI. Overall, sub-visible particles in the size range of ≥ (more than or equal to) 10 μm were less than 6,000 particles/mL. In the size range of ≥ (more than or equal to) 25 μm, the sub-visible particles were less than 600 particles/mL and were well within USP<788> limits even considering the increased sensitivity of using MFI for sub-visible particle detection. No significant differences were observed between Formulation 3 filled into Nipro PFS compared to Formulation 3 stored in glass vials with respect to sub-visible particles up to 24 months (data not shown), indicating that the stability profile of the formulation is not affected by contact with Nipro PFS.

TABLE 21
Sub-Visible Particles for Formulations 1-3 at 2-8° C.

| | SbVPs ≥2 μm (particles/ | | | SbVPs ≥10 μm (particles/ | | | SbVPs ≥25 μm (particles/ | | |
|---|---|---|---|---|---|---|---|---|---|
| Time | mL) at 2-8° C. | | | mL) at 2-8° C. | | | mL) at 2-8° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 1 | 34155 | 1008 | 1790 | 393 | 98 | 59 | 7 | 20 | 0 |
| 3 | 35353 | 1494 | 1521 | 225 | 52 | 19 | 3 | 0 | 3 |
| 6 | 38899 | 2398 | 10228 | 160 | 143 | 676 | 0 | 30 | 59 |
| 9 | 50016 | 3378 | 4969 | 533 | 204 | 251 | 25 | 32 | 37 |
| 12 | 54427 | 2452 | 4158 | 376 | 59 | 98 | 10 | 15 | 5 |
| 18 | 29704 | 1463 | 1330 | 184 | 68 | 55 | 17 | 9 | 6 |
| 24 | 20591 | 9289 | 11269 | 297 | 406 | 398 | 26 | 22 | 11 |
| 36 | 18716 | 2534 | 7578 | 354 | 212 | 297 | 107 | 48 | 39 |

SbVPs: Sub-visible particles

TABLE 22
Sub-Visible Particles for Formulations 1-3 at 25° C.

| | SbVPs ≥2 μm (particles/ | | | SbVPs ≥10 μm (particles/ | | | SbVPs ≥25 μm (particles/ | | |
|---|---|---|---|---|---|---|---|---|---|
| Time | mL) at 25° C. | | | mL) at 25° C. | | | mL) at 25° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 0.5 | 38785 | 1411 | 1086 | 216 | 120 | 89 | 7 | 12 | 22 |
| 1 | 26524 | 937 | 1369 | 246 | 69 | 30 | 17 | 10 | 5 |
| 2 | 34338 | 6140 | 2159 | 130 | 173 | 65 | 9 | 6 | 19 |
| 3 | 39657 | 1022 | 6339 | 114 | 37 | 96 | 6 | 6 | 12 |
| 6 | 15358 | 13316 | 14585 | 79 | 858 | 910 | 15 | 69 | 89 |
| 9 | 27019 | 4556 | 10106 | 209 | 240 | 683 | 32 | 21 | 61 |
| 12 | 62324 | 2256 | 10055 | 631 | 302 | 2061 | 7 | 37 | 268 |

SbVPs: Sub-visible particles

TABLE 23
Sub-Visible Particles for Formulations 1-3 at 40° C.

| | SbVPs ≥2 μm (particles/ | | | SbVPs ≥10 μm (particles/ | | | SbVPs ≥25 μm (particles/ | | |
|---|---|---|---|---|---|---|---|---|---|
| Time | mL) at 25° C. | | | mL) at 25° C. | | | mL) at 25° C. | | |
| (Months) | F1 | F2 | F3 | F1 | F2 | F3 | F1 | F2 | F3 |
| 0 | 39058 | 844 | 612 | 175 | 141 | 25 | 10 | 8 | 2 |
| 0.5 | 67190 | 740 | 2311 | 268 | 32 | 197 | 12 | 5 | 39 |
| 1 | 59549 | 1428 | 4399 | 511 | 32 | 241 | 5 | 5 | 15 |
| 2 | 42669 | 5810 | 7054 | 275 | 272 | 457 | 3 | 25 | 25 |
| 3 | 40732 | 1974 | 7686 | 170 | 86 | 488 | 9 | 0 | 25 |
| 6 | 29017 | 12221 | 12188 | 216 | 681 | 755 | 32 | 34 | 106 |

### Chemical Modifications to Primary Structure Using Peptide Mapping

Amino acids that could potentially undergo chemical modification over time and influence protein structure were monitored for Formulations 1-3. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and were monitored. However, as shown in Tables 24-26, no significant changes were observed in these modifications over time. Results were comparable between Formulation 3 filled into Nipro PFS compared to Formulation 3 stored in glass vials at the long-term storage condition (2-8° C.) up to 24 months with respect to % Met81 oxidation, % Asn317 deamidation and % Met254 oxidation (data not shown), indicating that the stability profile of the formulation is not affected by contact with Nipro PFS.

TABLE 24
% Met81 Oxidation in Formulations 1-3

| Time | % Met81 Oxidation at 2-8° C. | | | % Met81 Oxidation at 25° C. | | | % Met81 Oxidation at 40° C. | | |
|---|---|---|---|---|---|---|---|---|---|
| (Months) | F1 | F2 | F3 | F1 | F2 | F3 | F1 | F2 | F3 |
| 0 | 2.1 | 1.1 | 0.5 | 2.1 | 1.1 | 0.5 | 2.1 | 1.1 | 0.5 |
| 1 | 3.1 | 1.2 | 2.2 | 1.2 | 0.6 | 1.0 | 4.9 | 1.3 | 1.1 |
| 3 | 1.2 | 2.3 | 0.8 | 0.8 | 1.5 | 0.8 | 0.9 | 1.7 | 1.0 |
| 6 | 0.6 | 0.6 | 0.7 | 0.5 | 0.6 | 0.6 | 0.7 | 0.6 | 0.7 |
| 12 | 0.6 | 0.7 | 1.5 | 0.8 | 0.5 | 0.6 | NT | NT | NT |
| 18 | 0.8 | 0.5 | 0.6 | NT | NT | NT | NT | NT | NT |
| 24 | 1.0 | 0.9 | 1.0 | NT | NT | NT | NT | NT | NT |
| 36 | 0.3 | 0.3 | 0.4 | NT | NT | NT | NT | NT | NT |

NT: Test not performed;
F1: Formulation 1;
F2: Formulation 2;
F3: Formulation 3;
Met81: methionine residue 81 of TEV-48574

TABLE 25
% Asn317 Deamidation in Formulations 1-3

| | % Asn317 deamidation | | | % Asn317 deamidation | | | % Asn317 deamidation | | |
|---|---|---|---|---|---|---|---|---|---|
| Time | at 2-8° C. | | | at 25° C. | | | at 40° C. | | |
| (Months) | F1 | F2 | F3 | F1 | F2 | F3 | F1 | F2 | F3 |
| 0 | 7.8 | 7.4 | 7.8 | 7.8 | 7.4 | 7.8 | 7.8 | 7.4 | 7.8 |
| 1 | 8.6 | 7.7 | 8.7 | 7.6 | 7.4 | 7.5 | 8.0 | 7.6 | 8.3 |
| 3 | 7.1 | 7.9 | 7.4 | 8.0 | 8.1 | 6.6 | 7.9 | 7.7 | 7.2 |
| 6 | 8.4 | 7.0 | 6.8 | 7.2 | 6.0 | 5.0 | 7.0 | 7.3 | 8.7 |
| 12 | 9.6 | 9.8 | 9.1 | 8.9 | 9.7 | 10.0 | NT | NT | NT |
| 18 | 7.6 | 5.4 | 6.5 | NT | NT | NT | NT | NT | NT |
| 24 | 9.3 | 10.0 | 11.2 | NT | NT | NT | NT | NT | NT |
| 36 | 7.1 | 7.8 | 10.0 | NT | NT | NT | NT | NT | NT |

NT: Test not performed;
F1: Formulation 1;
F2: Formulation 2;
F3: Formulation 3;
Asn317: asparagine residue 317 of TEV-48574

TABLE 26
% Met254 Oxidation in Formulations 1-3

| | % Met254 Oxidation | | | % Met254 Oxidation | | | % Met254 Oxidation | | |
|---|---|---|---|---|---|---|---|---|---|
| Time | at 2-8° C. | | | at 25° C. | | | at 40° C. | | |
| (Months) | F1 | F2 | F3 | F1 | F2 | F3 | F1 | F2 | F3 |
| 0 | 3.7 | 2.4 | 1.5 | 3.7 | 2.4 | 1.5 | 3.7 | 2.4 | 1.5 |
| 1 | 4.9 | 2.5 | 3.9 | 2.3 | 2.2 | 2.5 | 7.4 | 4.4 | 3.9 |
| 3 | 2.5 | 4.3 | 2.3 | 1.9 | 4.2 | 3.1 | 1.9 | 9.5 | 8.7 |
| 6 | 1.8 | 2.3 | 2.4 | 1.8 | 4.3 | 4.5 | 1.8 | 18.8 | 20.5 |
| 12 | 1.7 | 2.6 | 3.9 | 1.9 | 6.4 | 5.6 | NT | NT | NT |
| 18 | 1.8 | 2.8 | 3.3 | NT | NT | NT | NT | NT | NT |
| 24 | 2.2 | 3.9 | 4.2 | NT | NT | NT | NT | NT | NT |
| 36 | 1.6 | 4.2 | 4.3 | NT | NT | NT | NT | NT | NT |

NT: Test not performed;
F1: Formulation 1;
F2: Formulation 2;
F3: Formulation 3;
Met254: methionine residue 254 of TEV-48574

### Potency by ELISA

The percent (%) potency of Formulations 1-3 was determined by enzyme-linked immunosorbent assay (ELISA). The results are shown in Table 27. No significant changes were observed between the formulations at standard storage conditions (2-8° C.), and the acceptance criteria of 70%-135% potency was met for up to 24 months and for up to 36 months. No significant difference was observed between Formulation 3 filled into Nipro PFS compared to Formulation 3 stored in glass vials with respect to % potency up to 24 months (data not shown), indicating that the stability profile of the formulation is not affected by contact with Nipro PFS.

TABLE 27
% Relative Potency of Formulations 1-3 by ELISA

| | % Potency at 2-8° C. | | | | | | | | |
|---|---|---|---|---|---|---|---|---|---|
| | Acceptance criteria: | | | | | | | | |
| Time | 70-135% | | | % Potency at 25° C. | | | % Potency at 40° C. | | |
| (Months) | F1 | F2 | F3 | F1 | F2 | F3 | F1 | F2 | F3 |
| 0 | 87 | 100 | 100 | 87 | 100 | 100 | 87 | 100 | 100 |
| 0.5 | NT | NT | NT | 92 | 100 | 90 | 95 | 95 | 94 |
| 1 | 101 | 97 | 98 | 99 | 90 | 96 | 104 | 103 | 98 |
| 2 | NT | NT | NT | 96 | 92 | 91 | 104 | 100 | 90 |
| 3 | 98 | 92 | 88 | 92 | 95 | 92 | 110 | 98 | 89 |
| 6 | 92 | 88 | 87 | 97 | 95 | 96 | 92 | 84 | 86 |
| 9 | 101 | 93 | 95 | 99 | 92 | 90 | NT | NT | NT |
| 12 | 89 | 95 | 106 | 108 | 103 | 105 | NT | NT | NT |
| 18 | 108 | 100 | 103 | NT | NT | NT | NT | NT | NT |
| 24 | 103 | 97 | 101 | NT | NT | NT | NT | NT | NT |
| 36 | 102 | 96 | 96 | NT | NT | NT | NT | NT | NT |

NT: Test not performed

Differential Scanning calorimetry (DSC) Analysis for Thermal Stability

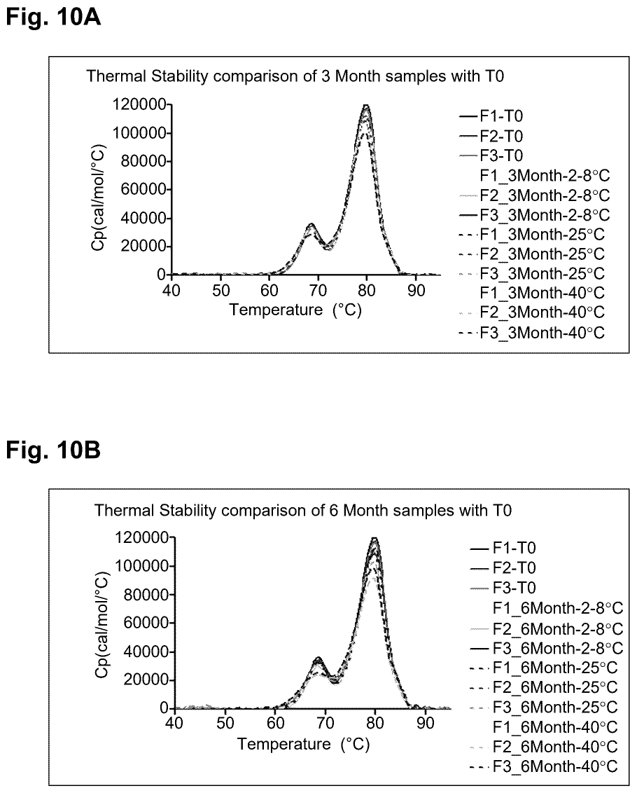

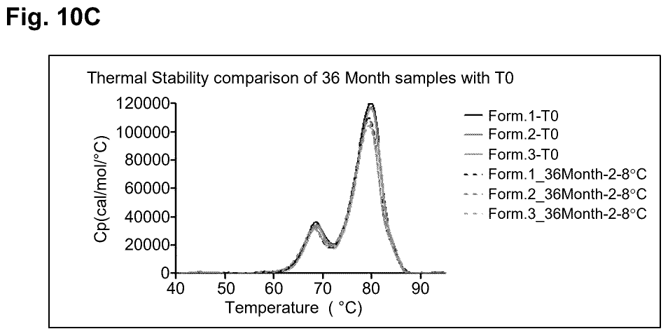

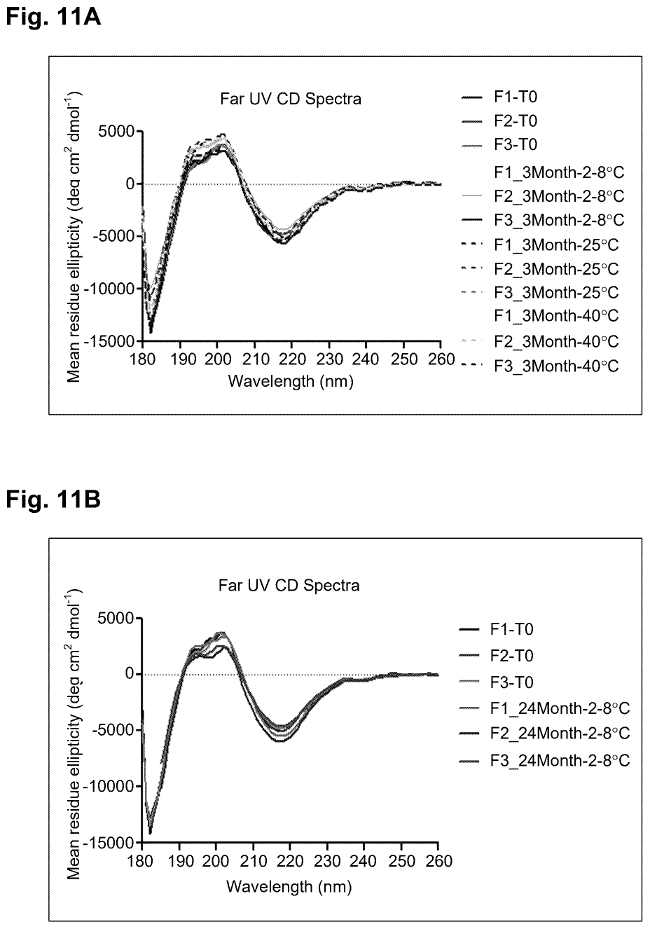

DSC was employed to evaluate thermal stability of Formulations 1-3. DSC analysis was performed for a time zero (T0) sample of Formulations 1-3 and for 3 month, 6 month, and 36 month samples at 2-8° C., 25° C. and 40° C. conditions. FIG. 10A (FIG. 10A) shows the thermal stability of Formulations 1-3 at time zero (T0) and at 2-8° C., 25° C., and 40° C. after 3 months. FIG. 10B (FIG. 10B) shows the thermal stability of Formulations 1-3 at time zero (T0) and at 2-8° C., 25° C., and 40° C. after 6 months. FIG. 10C (FIG. 10C) shows the thermal stability of Formulations 1-3 at time zero (T0) and at 2-8° C. after 36 months. No significant difference in thermal stability was observed for the three formulations at T0, 3 months, 6 months, and 36 months, as the transition temperatures (Tm) overlapped for all formulations. A small decrease in enthalpy was observed for formulations stored at accelerated and stressed conditions. This could be due to protein fragmentation observed in the samples at these conditions, leading to a decrease in enthalpy. No significant differences were observed between Formulation 3 filled into Nipro PFS compared to Formulation 3 stored in glass vials up to 6 months with respect to thermal stability, indicating that the stability profile of the formulation is not affected by contact with Nipro PFS.

### Secondary and Tertiary Protein Structure Analysis Using Circular Dichroism (CD) Spectroscopy

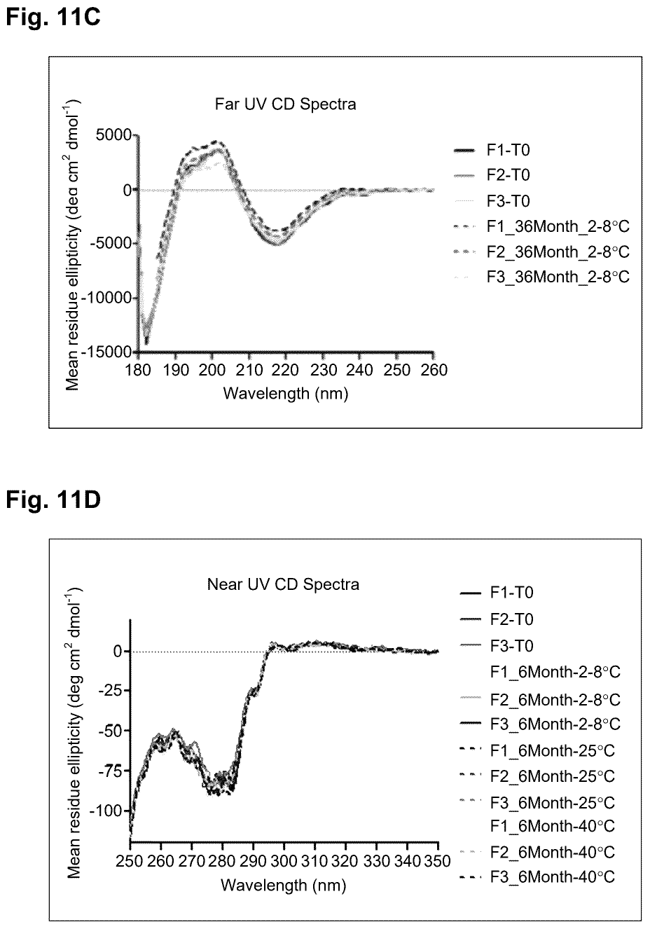

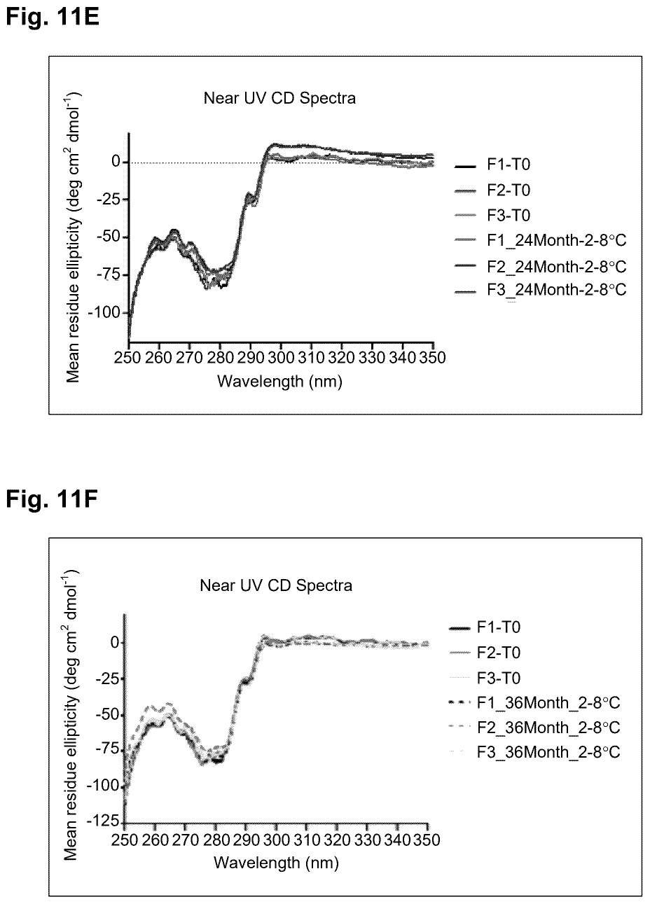

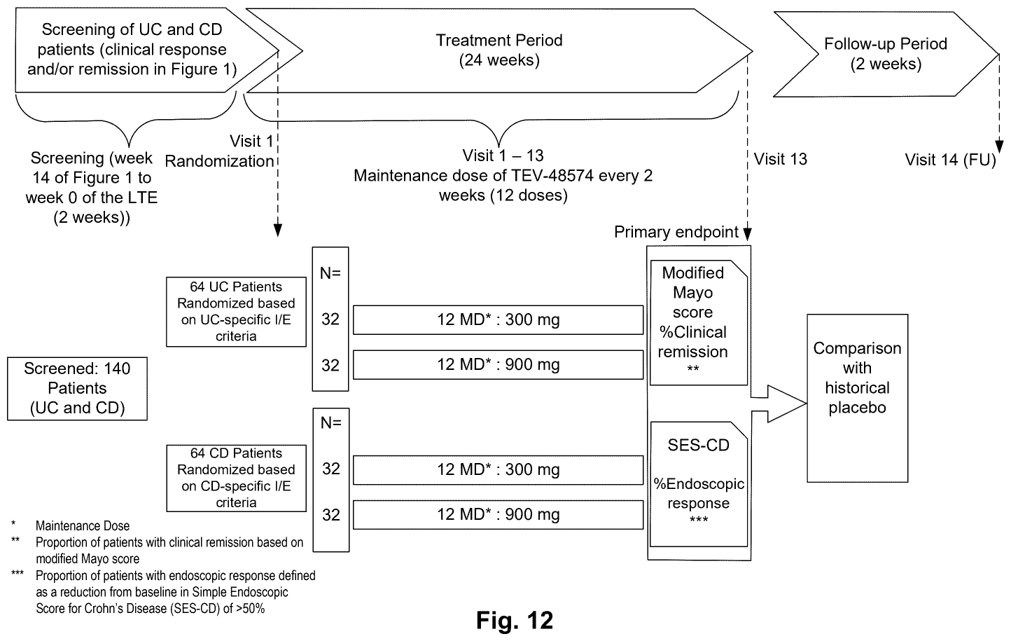

Secondary protein structure of Formulations 1-3 was analyzed using far-ultraviolet (far-UV) circular dichroism (CD), and tertiary protein structure of Formulations 1-3 was analyzed using near-UV CD. Secondary and tertiary structure analysis was performed for time zero (T0) samples of Formulations 1, 2 and 3; 3 month samples at 2-8° C., 25° C. and 40° C. conditions; 24 month samples at 2-8° C.; and 36 month samples at 2-8° C. The results are shown in FIGS. 11A-11F (FIGS. 11A-11F). Specifically, FIG. 11A shows the secondary structure of Formulations 1-3 at T0 and after 3 months at 2-8° C., 25° C. and 40° C. using far UV CD. FIG. 11B shows the secondary structure of Formulations 1-3 at T0 and after 24 months at 2-8° C. using far UV CD. FIG. 11C shows the secondary structure of Formulations 1-3 at T0 and after 36 months at 2-8° C. using far UV CD. FIG. 11D shows the tertiary structure of Formulations 1-3 at T0 and after 3 months at 2-8° C., 25° C. and 40° C. using near UV CD. FIG. 11E shows the tertiary structure of Formulations 1-3 at T0) and after 24 months at 2-8° C. using near UV CD. And, FIG. 11F shows the tertiary structure of Formulations 1-3 at T0) and after 36 months at 2-8° C. using near UV CD.

The far UV CD spectra showed negative maxima at around 217 nm, indicating beta sheet structure for T0 samples in all three formulations, which is expected for a monoclonal antibody. The near UV CD spectra showed positive maxima at around 292 nm, indicative of absorption by tryptophan residues and negative maxima at 276 nm, indicative of absorption by Tyrosine residues. No significant change in secondary structure or tertiary structure was observed for the three formulations up to 3 months. At 24 months and 36 months, small changes in CD spectra were observed at around 200 nm. This could be due to protein fragmentation since the absorption in this region is predominantly due to peptide bond absorption. However, no significant change in secondary structure of protein (absorption at 217 nm) was observed.

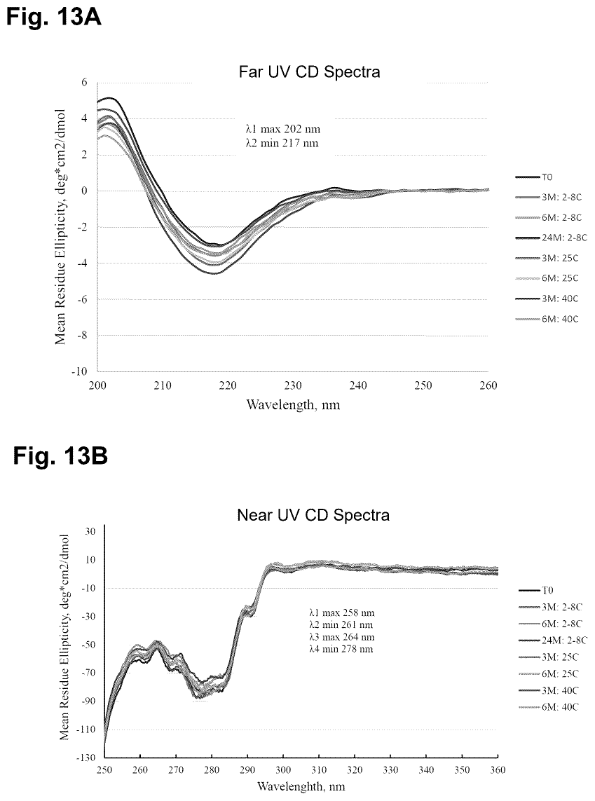

FIG. 13A shows the secondary structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using far UV CD. FIG. 13B shows the tertiaty structure of Formulation 3 stored in Nipro prefilled syringes (PFS) at time zero (T0) and after 3 months (3M), 6 months (6M), and 24 months (24M) of storage at 2-8° C., and after 3 months (3M) and 6 months (6M) of storage at 25° C. and 40° C. using near UV CD. The spectra obtained for the Nipro PFS samples are comparable to those obtained for Formulation 3 stored in glass vials (FIGS. 11A-11B and 11D-11E).

### Polysorbate 80 Analysis

Polysorbate 80 (PS80) as a surfactant/excipient present in Formulations 1-3 could potentially undergo degradation, which results in reactive peroxides that over the shelf life of the product may impact protein stability. PS80 analysis was performed on Formulations 1-3 after storage for 24 months and 36 months at 2-8° C. by testing PS80 levels. As shown in Table 28, the measured PS80 levels were 0.02% (w/v) for 24 month samples in all formulations, correlating with the PS80 concentration expected. At 36 months, PS80 level in formulations 2 and 3, which are liquid formulation, showed a decrease. No significant differences were observed between Formulation 3 filled into Nipro PFS compared to Formulation 3 stored in glass vials with respect to PS80 levels at 24 months, indicating that the stability profile of the formulation is not affected by contact with Nipro PFS.

TABLE 28
Detected Polysorbate 80 (PS80) Levels in Formulations 1-3
After Storage for 24 Months and 36 Months at 2-8° C.

| Formulation | PS80 levels | PS80 levels |
|---|---|---|
| at 2-8° C. | (w/v) at 24 M | (w/v) at 36 M |
| Formulation 1 | 0.02% | 0.02% |
| Formulation 2 | 0.02% | 0.01% |
| Formulation 3 | 0.02% | 0.01% |

### Conclusions

The stability of TEV-48574 in lyophilized form (Formulation 1 at 100 mg/mL) was comparable to liquid formulations at 100 mg/mL (Formulation 2) and 150 mg/mL (Formulation 3) stored at 2-8° C., the standard storage condition for the drug product. At accelerated (25° C.) and stressed (40° C.) conditions, both liquid formulations showed protein fragmentation as the primary degradation pathway, which was not observed for lyophilized Formulation 1. However, this is an expected observation considering that lyophilized formulations are more stable compared to liquid formulations.

Formation of acidic species was observed in liquid formulations compared to the lyophilized formulation, measured using icIEF, and was concomitant with protein fragmentation observed using SEC and CGE. This indicates deamidation of certain amino acid residues, potentially causing fragmentation. See Wang, W. et al., J. Pharm. Sci., 96:1-26 (2007).

Based on these data, lyophilized Formulation 1 can be bridged to liquid formulations at 100 mg/mL and 150 mg/mL, having a comparable stability profile and meeting acceptance criteria for up to 24 months and up to 36 months at 2-8° C. storage conditions.

Furthermore, the stability of TEV-48574 at 150 mg/mL (Formulation 3) stored in Nipro PFS was found to be comparable as Formulation 3 samples stored in glass vials. Based on this data, 24 month storage at 2-8° C., the standard storage condition for the drug product, is compatible with the Nipro PFS.

### Example 4: Further Clinical Evaluation of TEV-48574 in Moderate to Severe Ulcerative Colitis or Moderate to Severe Crohn's Disease

TEV-48574 is assessed in a 24-week Phase 2b, randomized, double-blind long-term extension (LTE) study to evaluate the pharmacokinetics, efficacy, safety, and tolerability of TEV-48574 in adult patients with moderate to severe ulcerative colitis (UC) or Crohn's disease (CD) who completed the treatment phase of the 14-week study described in Example 1 above. The study schematic diagram is presented in FIG. 12.

Eligible patients meet all of the following criteria:
Adults of male and female sex (without restrictions on gender) who at the time of informed consent achieved clinical response and/or clinical remission in the 14-week study of Example 1.
   Clinical response assessed by decrease from baseline in modified (9-point rectal bleeding, stool frequency, and endoscopy) Mayo score by ≥2 points and at least 30%, with a decrease in rectal bleeding subscore of ≥1 point or an absolute subscore of 0 or 1 at week 14 of the of the Example 1 study in patients with moderate to severe UC.
   Clinical remission at week 14 of the study of Example 1 in patients with moderate to severe UC. Clinical remission is a modified (9-point rectal bleeding, stool frequency, and endoscopy) Mayo score of ≤2 points, which is defined by:
      stool frequency subscore of 0 or 1,
      rectal bleeding subscore of 0, and
      endoscopic subscore of 0 or 1, where a score of 1 does not include “friability.”
   Clinical response assessed by ≥100-point decrease in Crohn's Disease Activity Index (CDAI) score from baseline week 14 of the study in Example 1 in patients with moderate to severe CD.
   Clinical remission defined as a CDAI score <150 at week 14 of the study in Example 1 in patients with moderate to severe CD.

Patients who meet all the inclusion criteria are randomly assigned to receive TEV-48574 (12 maintenance doses): 900 mg or 300 mg in a 1:1 ratio, stratified by diagnosis (UC or CD).

### General Design

The study includes a 24-week treatment period and a 2-week follow-up period.

Randomization: Patients satisfying the eligibility criteria at the end of treatment visit (EOT) in the 14-week study described in Example 1 above are randomized in a 1:1 ratio (stratified by diagnosis [UC or CD], to 1 of 2 treatment groups for the double-blind treatment period (See Table 29 below).

All study treatments are administered in a double-blind fashion as 1 SC infusion Q2W.

TABLE 29
Randomization Scheme

| Randomization | Planned approximate | |
|---|---|---|
| ratio | number randomized | Treatment (Q2W) |
| 1 | 32 UC + 32 CD | TEV-48574 300 mg |
| | | (12 maintenance doses) |
| 1 | 32 UC + 32 CD | TEV-48574 900 mg |
| | | (12 maintenance doses) |

CD = Crohn's disease; Q2W = every 2 weeks; UC = ulcerative colitis.

During the 24-week treatment period, patients visit the site Q2W on days 1, 15, 29, 43, 57, 71, 85, 99, 113, 127, 141 and 155 (±3 days) for TEV-48574 administration (12 visits) and day 169 (±3 days) for endpoints assessment. After the end of the 24-week treatment period, patients return to the site for a follow-up visit (day 183 [±14 days]). Patients who complete the follow-up visit are considered to have completed the study.

Patients are allowed to use the following medications as detailed below:
Concomitant use of oral 5-ASA or sulfasalazine. Dose must be stable through the transition period and throughout the study (week 24).
A stable dose of oral corticosteroids (prednisone equivalent of up to 20 mg/day; budesonide of up to 9 mg/day) through the transition period and throughout the study (week 24).
A stable dose of immunosuppressant drugs (methotrexate≤25 mg intramuscular or SC once weekly or ≤15 mg per os once weekly; 6-MP≤1.5 mg/kg/day; or AZA ≤2.5 mg/kg/day).

The following endpoints are analyzed to demonstrate a lack of immunogenicity:
Treatment-emergent anti-drug antibody (ADA) results and responses: change from baseline (Example 1 study) and throughout the study
Neutralizing ADA in ADA positive patients throughout the study
Impact of the presence of ADAs on pharmacokinetics and clinical safety will be assessed if applicable.

The following measures/parameters are analyzed to demonstrate safety and tolerability:
Frequency of adverse events
Change from baseline (LTE baseline) in clinical laboratory test results (serum chemistry, hematology, and urinalysis)
Change from baseline (LTE baseline) in vital signs measurements (blood pressure, pulse rate, body temperature, and respiratory rate)
Change from baseline (LTE baseline) in 12-lead electrocardiogram (ECG) findings
Use of concomitant medication
Number (%) of patients who stopped the TEV-48574 due to adverse events
Local tolerability at the injection site
Device-related adverse events and malfunctions (for the commercial SC infusion system).

The following efficacy endpoints are analyzed to demonstrate efficacy of TEV-48574:
Clinical remission based on modified (9-point rectal bleeding, stool frequency, and endoscopy) Mayo score of ≤2 points defined by a stool frequency subscore of 0 or 1, rectal bleeding subscore of 0, and an endoscopic subscore of 0 or 1, where a score of 1 does not include “friability” at week 24 in patients with UC
Endoscopic response, defined as a decrease in Simple Endoscopic Score for Crohn's Disease (SES-CD) of at least 50% from the Example 1 study baseline at week 24 in patients with CD.

The following endpoints are also analyzed to demonstrate efficacy of TEV-48574:
Clinical response, based on modified (9-point rectal bleeding, stool frequency, and endoscopy) Mayo score of at least 2 points AND at least a 30% reduction from the Example 1 study baseline with either a decrease in rectal bleeding subscore of at least 1 or an absolute rectal bleeding subscore of less than or equal to 1 at week 24 in patients with UC
Endoscopic improvement from the Example 1 study baseline based on Mayo endoscopic subscore of 0 or 1 at week 24 in patients with UC
Endoscopic remission based on Mayo endoscopic subscore of 0 at week 24 in patients with UC
Clinical response defined as decrease from the Example 1 study baseline of at least 50% in 2-item patient-reported outcome (PRO2; rectal bleeding and stool frequency) at weeks 0, 4, 8, 10, 12, 16, 20, and 24 in patients with UC
Clinical remission based on PRO2 (rectal bleeding-0 and stool frequency=0) at weeks 0, 4, 8, 10, 12, 16, 20 and 24 in patients with UC
Histological remission based on Robarts Histopathology Index (RHI≤5) at week 24 in patients with UC
Histological remission based on Geboes index score≤3.1 in patients with UC
Clinical response based on Crohn's Disease Activity Index (CDAI): ≥100-point decrease in CDAI score from the Example 1 study baseline at week 24 in patients with CD at weeks 0, 4, 8, 10, 12, 16, 20 and 24
Clinical remission based on CDAI score <150 at week 0, 4, 8, 10, 12, 16, 20 and 24 in patients with CD
Endoscopic remission based on SES-CD: SES-CD score of 0-2, or SES-CD score of 0-4, with no individual sub score >1 at week 24 in patients with CD
Clinical response defined as decrease in PRO2 from the Example 1 study baseline of at least 50% in PRO2 at weeks 0, 4, 8, 10, 12, 16, 20 and 24 in patients with CD
Clinical remission based on PRO2 (abdominal pain≤1 and stool frequency≤3) at weeks 0, 4, 8, 10, 12, 16, 20 and 24 in patients with CD
Endoscopic response, defined as a decrease in modified multiplier (MM)-SES-CD of >50% from the Example 1 study baseline at week 24 in patients with CD
Histologic response (≥50% decrease) in Global Histologic Activity Score from the Example 1 study baseline at week 24 in patients with CD.

### Example 5: Stability of TEV-48574 Liquid Formulation at 200 mg/ml

The purpose of this study was to evaluate the long term stability of a high concentration liquid TEV-48574 formulation in comparison to a lyophilized drug product. Two formulations were tested: a lyophilized form containing 100 mg/mL of drug product in 10 mM Histidine, 5% (w/v) Sucrose, 100 mM Arg-HCl, 0.02% (w/v) PS-80 at pH 6.0 (Formulation 4 or F4), and a liquid form containing 200 mg/mL of drug product in 10 mM Histidine, 5% (w/v) Sucrose, 100 mM Arg-HCl, 0.02% (w/v) PS-80 at pH 6.0 (Formulation 5 or F5). The stability of these formulations was evaluated in 5 cc vials with a 20 mm size stopper at the long-term storage condition of 2-8° C. The impact of these conditions on several product quality attributes was tested for the formulations.

### Visual Appearance, Protein Concentration, Osmolality and Viscosity

The results of a visual appearance, protein concentration, and osmolality analysis for Formulations 4 and 5 are shown in Tables 30-31. At the long-term storage condition of 2-8° C., no significant difference across the formulations was observed.

The measured protein concentration for both formulations at the long-term storage condition of 2-8° C. were close to the nominal concentration expected and did not vary over time. Osmolality and viscosity of the drug products were evaluated at time zero (T0). The osmolality and viscosity of the 200 mg/mL liquid formulation (F5) were observed to be higher than those observed for the lyophilized formulation (F4) at 100 mg/mL.

TABLE 30
Visual Appearance and Protein Concentration Results at 2-8° C.

| | | | | Protein concentration | |
|---|---|---|---|---|---|
| Time | | Visual appearance | | (mg/mL) | |
| Point | | F4 | F5 | F4 | F5 |
| T0 | | L; C; FFVP | SP; O; FFVP | 95.8 | 201.4 |
| 1 | M | NT | NT | 94.6 | 198.8 |
| 3 | M | L; S; FFVP | L; C; FFVP | NT | NT |
| 6 | M | L; S; FFVP | L; S; FFVP | 95.6 | 200.4 |
| 9 | M | L; V; FFVP | VSY; S; | 97.6 | 194.3 |
| | | | FFVP | | |
| 12 | M | L; C; FFVP | L; C; FFVP | 97.1 | 197.9 |
| 18 | M | L; C; FFVP | L; C; FFVP | 96.8 | 199.6 |
| 24 | M | L; C; FFVP | L; C; FFVP | 96.5 | 201.0 |

T0: Time zero;
M: Month(s);
F4: Formulation 4;
F5: Formulation 5;
S: Slightly opalescent;
O: Opaslescent;
V: Very opaslescent;
L: Colorless;
FFVP: Free from visible particles;
C: Clear;
VSY: Very slightly yellow;
SP: Slightly pink;
NT: Not tested

TABLE 31
Osmolality and Viscosity Results at 2-8° C.

| | | |
|---|---|---|
| | Osmolality | Viscosity (cP) |
| Time | (mOsm/kg) | measured at 20° C. |

| Point | F4 | F5 | F4 | F5 |
|---|---|---|---|---|
| T0 | 382 | 542 | 3 | 19 |

T0: Time zero;
F4: Formulation 4;
F5: Formulation 5

### Size Exclusion Chromatography (SEC)

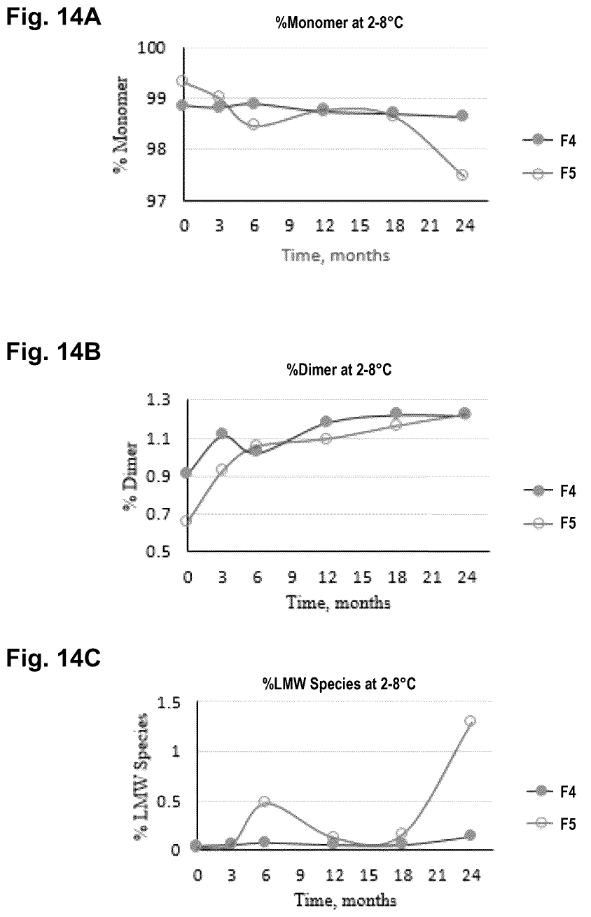

Table 32 and FIGS. 14A-14C show the percent (%) monomer, % dimer and % low molecular weight species of Formulations 4 and 5 measured by SEC. Both formulations stored at the long-term storage conditions of 2-8° C. met the acceptance criteria of % monomer and % dimer levels for up to 24 months. A slight decrease in % monomer with concurrent increase in % low molecular weight species was observed for Formulation 5 compared to Formulation 4.

TABLE 32
% Monomer, % Dimer and % Low Molecular Weight Species at 2-8° C.

| | Formulation 4 | | | Formulation 5 | | |
|---|---|---|---|---|---|---|
| | % | | | % | | |
| | Monomer | % Dimer | | Monomer | % Dimer | |
| Time | (Acceptance | (Acceptance | % | (Acceptance | (Acceptance | % |
| (Months) | criteria: ≥95.0%) | criteria: ≤5.0%) | LMW | criteria: ≥95.0%) | criteria: ≤5.0%) | LMW |
| 0 | 98.85 | 0.91 | 0.04 | 99.32 | 0.65 | 0.02 |
| 1 | NT | NT | NT | NT | NT | NT |
| 3 | 98.81 | 1.11 | 0.05 | 99.01 | 0.92 | 0.05 |
| 6 | 98.88 | 1.02 | 0.07 | 98.45 | 1.05 | 0.47 |
| 9 | NT | NT | NT | NT | NT | NT |
| 12 | 98.73 | 1.18 | 0.05 | 98.77 | 1.09 | 0.12 |
| 18 | 98.69 | 1.22 | 0.05 | 98.64 | 1.16 | 0.16 |
| 24 | 98.63 | 1.21 | 0.13 | 97.45 | 1.22 | 1.29 |

LMW: Low molecular weight species;
NT: Not tested

### Capillary Gel Electrophoresis (Reducing and Non-Reducing)

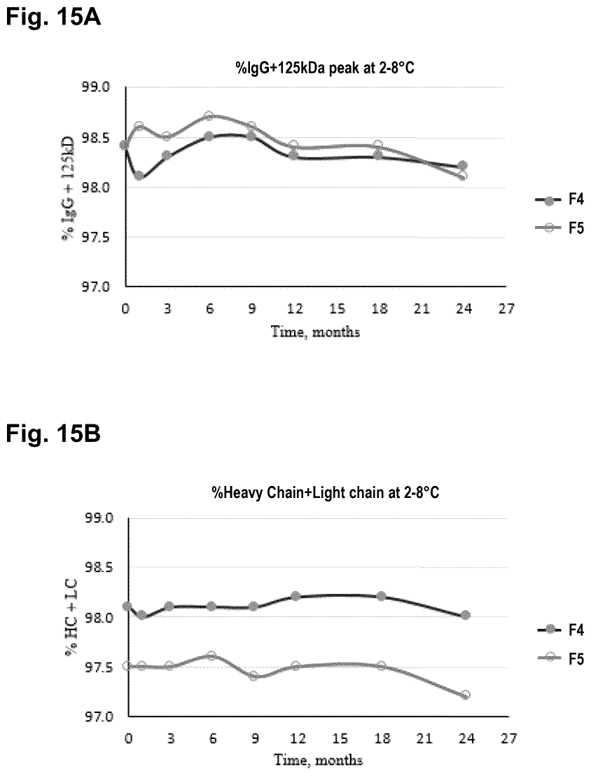

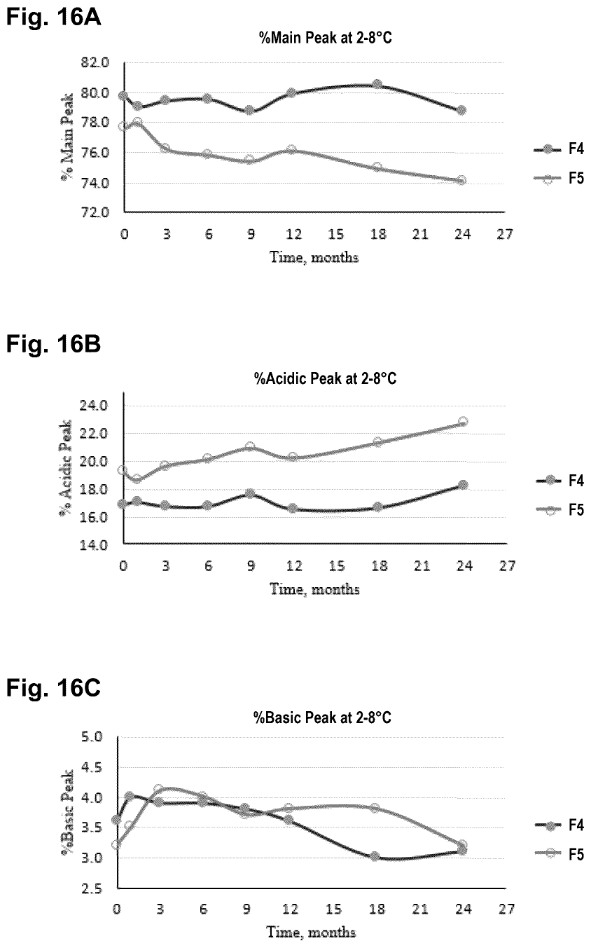

The percent (%) immunoglobulin G (IgG)+125 kDa peak was determined for Formulations 4 and 5 using non-reducing capillary gel electrophoresis (CGE). In addition, the % heavy chain+light chain was determined for Formulations 4 and 5 using reducing CGE. These results are shown in Tables 33-34 and FIGS. 15A-15B, respectively. In both formulations stored at the long-term storage conditions of 2-8° C., the % purity met acceptance criteria for up to 24 months.

TABLE 33
% IgG + 125 kDa Peak Measured Using Non-Reducing
CGE for Formulations 4 and 5 at 2-8° C.

| | | |
|---|---|---|
| Time | F4 | F5 |

| | |
|---|---|
| (Months) | 2-8° C.; Acceptance criteria: ≥90.0% |

| | | |
|---|---|---|
| 0 | 98.4 | 98.4 |
| 1 | 98.1 | 98.6 |
| 3 | 98.3 | 98.5 |
| 6 | 98.5 | 98.7 |
| 9 | 98.5 | 98.6 |
| 12 | 98.3 | 98.4 |
| 18 | 98.3 | 98.4 |
| 24 | 98.2 | 98.1 |

TABLE 34
% Heavy Chain and Light Chain Measured Using Reducing
CGE for Formulations 4 and 5 at 2-8° C.

| | | |
|---|---|---|
| Time | F4 | F5 |

| | |
|---|---|
| (Months) | 2-8° C.; Acceptance criteria: ≥90.0% |

| | | |
|---|---|---|
| 0 | 98.1 | 97.5 |
| 1 | 98.0 | 97.5 |
| 3 | 98.1 | 97.5 |
| 6 | 98.1 | 97.6 |
| 9 | 98.1 | 97.4 |
| 12 | 98.2 | 97.5 |
| 18 | 98.2 | 97.5 |
| 24 | 98.0 | 97.2 |

Capillary Isoelectric Focusing (icIEF)

The percent (%) content of main species (main peak), % acidic species (acidic peak), and % basic species (basic peak) of Formulations 4 and 5 (F4 and F5) were measured using capillary isoelectric focusing (icIEF). The results are shown in Table 35. An overlay of stability trends in charge heterogeneity is presented in FIGS. 16A-16C. At the long-term storage conditions of 2-8° C. (FIG. 16A, 16B, and 16C), both formulations (F4 and F5) met acceptance criteria for 24 months.

TABLE 35
% Main Peak, % Acidic Species and % Basic Species Determined
Using icIEF for Formulations 1 and 2 at 2-8° C.

| | Formulation 4 at 2-8° C. | | | Formulation 5 at 2-8° C. | | |
|---|---|---|---|---|---|---|
| | | % Acidic | % Basic | | % Acidic | % Basic |
| | % Main peak | species | species | % Main peak | species | species |
| | (Acceptance | (Acceptance | (Acceptance | (Acceptance | (Acceptance | (Acceptance |
| Time | criteria: | criteria: | criteria: | criteria: | criteria: | criteria: |
| (Months) | 50-90%) | 10-40%) | 0-10%) | 50-90%) | 10-40%) | 0-10%) |
| 0.0 | 79.7 | 16.8 | 3.6 | 77.6 | 19.2 | 3.2 |
| 1.0 | 79.0 | 17.0 | 4.0 | 77.9 | 18.6 | 3.5 |
| 3.0 | 79.4 | 16.7 | 3.9 | 76.2 | 19.6 | 4.1 |
| 6.0 | 79.5 | 16.7 | 3.9 | 75.8 | 20.1 | 4.0 |
| 9.0 | 78.7 | 17.5 | 3.8 | 75.4 | 20.9 | 3.7 |
| 12.0 | 79.9 | 16.5 | 3.6 | 76.1 | 20.2 | 3.8 |
| 18.0 | 80.4 | 16.6 | 3.0 | 74.9 | 21.3 | 3.8 |
| 24.0 | 78.7 | 18.2 | 3.1 | 74.1 | 22.7 | 3.2 |

### Sub-Visible Particles Using Micro-Flow Imaging (MFI)

Sub-visible particles in Formulations 4 and 5 were measured at different time points at the long-term storage condition of 2-8° C. The results are shown in Table 36. No significant changes in sub-visible particles were observed. Overall, sub-visible particles in the size range of ≥ (more than or equal to) 10 μm were less than 6,000 particles/mL. In the size range of ≥ (more than or equal to) 25 μm, the sub-visible particles were less than 600 particles/mL and were well within USP<788> limits even considering the increased sensitivity of using MFI for sub-visible particle detection. This data indicates that the 200 mg/mL formulation (F5) does not have a significant impact on sub-visible particle count at the long-term-storage condition.

TABLE 36
Sub-Visible Particles for Formulations 4 and 5 at 2-8° C.

| | SbVPs ≥ 2 μm | | SbVPs ≥ 10 μm | | SbVPs ≥ 25 μm | |
|---|---|---|---|---|---|---|
| | (particles/mL) | | (particles/mL) | | (particles/mL) | |
| Time | at 2-8° C. | | at 2-8° C. | | at 2-8° C. | |
| (Months) | F4 | F5 | F4 | F5 | F4 | F5 |
| 0 | 32581 | 2658 | 417 | 404 | 65 | 57 |
| 1 | 31819 | NT | 262 | NT | 13 | NT |
| 3 | 33888 | 4513 | 299 | 354 | 39 | 57 |

SbVPs: Sub-visible particles;
NT: Not tested

### Chemical Modifications to Primary Structure Using Peptide Mapping

Amino acids that could potentially undergo chemical modification over time and influence protein structure were monitored for Formulations 4 and 5. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and was monitored. As shown in Table 37, no significant changes were observed in % Met81 and % Met254 oxidation over time at the long-term storage condition of 2-8° C. for 24 months. At the 24M timepoint, the liquid drug product (F5) showed higher levels of Asn317 deamidation compared to the lyophilized drug product (F4). This difference in the extent of Asn deamidation may be the result of lower stability of the liquid drug product compared to the lyophilized drug product, and may not be attributable to the high nominal protein concentration in F5.

TABLE 37
% Met81 Oxidation, % Asn317 Deamidation, and
% Met254 Oxidation in Formulations 4 and 5

| | | | |
|---|---|---|---|
| | % Met81 | % Asn317 | % Met254 |
| | Oxidation | deamidation | Oxidation |
| Time | at 2-8° C. | at 2-8° C. | at 2-8° C. |

| (Months) | F4 | F5 | F4 | F5 | F4 | F5 |
|---|---|---|---|---|---|---|
| 0 | 0.67 | 0.49 | 6.80 | 7.01 | 1.79 | 1.85 |
| 1 | 0.46 | 0.60 | 8.22 | 6.62 | 1.56 | 2.07 |
| 3 | 1.44 | 0.87 | 6.08 | 6.02 | 2.73 | 2.47 |
| 6 | 1.25 | 0.80 | 6.54 | 7.48 | 2.68 | 2.51 |
| 12 | 0.94 | 0.51 | 6.64 | 4.63 | 2.19 | 2.11 |
| 18 | 0.87 | 0.77 | 6.66 | 6.22 | 2.35 | 2.90 |
| 24 | 0.4 | 1.0 | 7.6 | 11.7 | 2.3 | 3.8 |

F4: Formulation 4;
F5: Formulation 5;
Met81: methionine residue 81 of TEV-48574;
Asn317: asparagine residue 317 of TEV-48574;
Met254: methionine residue 254 of TEV-48574

### Potency by ELISA

The percent (%) potency of Formulations 4 and 5 was determined by enzyme-linked immunosorbent assay (ELISA). The results are shown in Table 38. No significant changes were observed between the formulations at the long-term storage condition (2-8° C.), and the acceptance criteria of 70%-135% potency was met for 24 months.

TABLE 38
% Relative Potency of Formulations 4 and 5 by ELISA

| | |
|---|---|
| | % Potency at 2-8° C. |
| Time | Acceptance criteria: 70-135% |

| (Months) | F4 | F5 |
|---|---|---|
| 0 | 110 | 113 |
| 1 | 118 | 112 |
| 3 | 108 | 100 |
| 6 | 99 | 96 |
| 9 | 107 | 108 |
| 12 | 106 | 99 |
| 18 | 100 | 92 |
| 24 | 98 | 92 |

### Secondary and Tertiary Protein Structure Analysis Using Circular Dichroism (CD) Spectroscopy

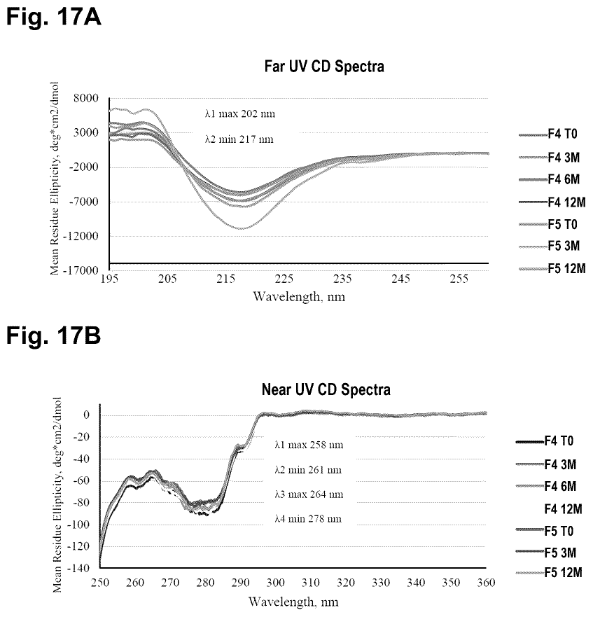

Secondary protein structure of Formulations 4 and 5 was analyzed using far-ultraviolet (far-UV) circular dichroism (CD), and tertiary protein structure of Formulations 4 and 5 was analyzed using near-UV CD. Secondary structure analysis was performed for time zero (T0) samples, and 3 month, 6 month, and 12 month samples of Formulation 4 at 25° C.; and for T0 samples, and 3 month and 12 month samples of Formulation 5 at 25° C. Tertiary structure analysis was performed for time zero (T0) samples, and 3 month and 12 month samples of Formulation 4 at 25° C.; and for T0 samples, and 3 month, 6 month and 12 month samples of Formulation 5 at 25° C. The results are shown in FIGS. 17A-17B (FIGS. 17A-17B). Specifically, FIG. 17A shows the secondary structure of Formulation 4 (F4) at time zero (T0) and after 3 months (3M), 6 months (6M) and 12 months (12M) of storage at 25° C., and of Formulation 5 (F5) at T0, and after 3M and 12M of storage at 25° C. using far UV CD. FIG. 17B shows the tertiary structure of F4 at T0, and after 3M and 12M of storage at 25° C., and of F5 at T0, and after 3M, 6M and 12M of storage at 25° C. using near near UV CD. No significant change in secondary structure or tertiary structure was observed for either the lyophilized formulation (F4) or high concentration liquid formulation (F5).

### Conclusions

The stability of TEV-48574 in lyophilized form (Formulation 4 at 100 mg/mL) was comparable to a liquid formulation at 200 mg/mL (Formulation 5). At the long-term storage condition of 2-8° C., the drug product stability data for both formulations were observed to be comparable.

At the 24M timepoint, the liquid drug product showed higher levels of Asn317 deamidation compared to the lyophilized drug product. This difference in the extent of Asn deamidation may be the result of lower stability of the liquid drug product compared to the lyophilized drug product, and may not be attributable to the high nominal protein concentration in F2. A slight decrease in % monomer with concurrent increase in % low molecular weight species was also observed for F5 compared to F4. However, lyophilized formulations are typically more stable compared to liquid formulations.

Based on these data, the 200 mg/mL high concentration liquid drug product was observed to be stable at the 2-8° C., with no significant impacts on critical quality attributes of TEV-48574.

### Example 6: Evaluation of Arginine-HCl as an Excipient in TEV-48574 Liquid Formulation

The purpose of this study was to evaluate the effectiveness of Arginine-HCl (Arg-HCl) as an excipient and its impact on TEV-48574 drug product stability. Four liquid formulations were tested. Two formulations were prepared in 10 mM Histidine, 5% (w/v) Sucrose, 100 mM Arg-HCl, 0.02% (w/v) PS-80 at pH 6.0 with drug product at either 150 mg/mL (Formulation 1A) or 100 mg/mL (Formulation 2A). Two additional formulations were prepared in 10 mM Histidine, 5% (w/v) Sucrose, 0.02% (w/v) PS-80 at pH 6.0 with drug product at either 150 mg/mL (Formulation 1B) or 100 mg/mL (Formulation 2B). The stability of these formulations was evaluated at the stressed storage condition of 40° C. The impact of these conditions on product quality attributes was evaluated.

### Visual Appearance, Protein Concentration, Osmolality, and Viscosity

The results of a visual appearance, protein concentration, and osmolality analysis for Formulations 1A-2A and 1B-2B are shown in Tables 39-41. At the stressed storage condition of 40° C., the visual appearance for formulations 1A and 1B was observed to be slightly yellow, potentially related to degradation of these formulations at elevated temperatures over time. The measured protein concentration for the 4 formulations at 40° C. were close to the nominal concentration expected and did not vary over time. Osmolality and viscosity of the drug products was evaluated, and no significant difference was observed over time.

TABLE 39
Visual Appearance Results at 40° C.

| Time | Visual Appearance | | | |
|---|---|---|---|---|
| Point | 1A | 1B | 2A | 2B |
| T0 | L; FFVP; S | L; FFVP; S | L; FFVP; S | L; FFVP; S |
| 1 WK | L; FFVP; O | L; FFVP; S | L; FFVP; S | L; FFVP; S |
| 2 WK | L; FFVP; O | L; FFVP; O | L; FFVP; O | L; FFVP; O |
| 3 WK | L; 1 fibrous | L; FFVP; O | L; FFVP; S | L; FFVP; S |
| | particle; O | | | |
| 4 WK | L; FFVP; O | L; FFVP; S | L; FFVP; S | L; FFVP; S |
| 6 WK | L; FFVP; O | L; FFVP; O | L; FFVP; O | L; FFVP; O |
| 8 WK | VSY; FFVP; O | SY; FFVP; O | L; FFVP; O | L; FFVP; O |

T0: Time zero;
WK: Weeks;
1A: Formulation 1A;
1B: Formulation 1B;
2A: Formulation 2A;
2B: Formulation 2B;
L: Colorless;
FFVP: Free from visible particles;
O: Opaslescent;
SY: Slightly yellow;
VSY: Very slightly yellow;
S: Slightly opalescent

TABLE 40
Protein Concentration Results at 40° C.

| | |
|---|---|
| Time | Protein concentration (mg/mL) |

| Point | 1A | 1B | 2A | 2B |
|---|---|---|---|---|
| T0 | 152.4 | 150.7 | 102.7 | 104.9 |
| 1 WK | 150.7 | 152.2 | 102.8 | 111.6 |
| 2 WK | 150.3 | 149.0 | 103.1 | 108.5 |
| 3 WK | 150.8 | 150.1 | 103.7 | 108.4 |
| 4 WK | 148.7 | 150.2 | 107.7 | 107.8 |
| 6 WK | 147.5 | 148.5 | 101.4 | 107.5 |
| 8 WK | 148.4 | 150.8 | 104.7 | 106.5 |

T0: Time zero;
WK: Weeks;
1A: Formulation 1A;
1B: Formulation 1B;
2A: Formulation 2A;
2B: Formulation 2B

TABLE 41
Osmolality Results at 40° C.

| | |
|---|---|
| Time | Osmolality (mOsm/kg) |

| Point | 1A | 1B | 2A | 2B |
|---|---|---|---|---|
| T0 | 444 | 222 | 365 | 179 |
| 1 WK | 427 | 231 | 359 | 180 |
| 2 WK | 416 | 226 | 362 | 182 |
| 3 WK | 423 | 216 | 366 | 181 |
| 4 WK | 427 | 234 | 366 | 182 |
| 6 WK | 420 | 223 | 373 | 189 |
| 8 WK | 434 | 225 | 359 | 189 |

T0: Time zero;
WK: Weeks;
1A: Formulation 1A;
1B: Formulation 1B;
2A: Formulation 2A;
2B: Formulation 2B

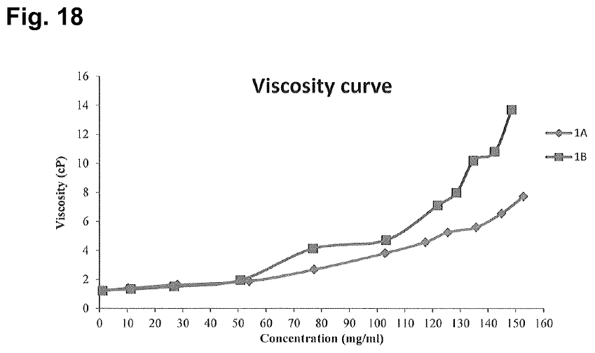

Osmolality of formulations 1A and 2A, which contain Arg-HCl, was higher compared to formulations 1B and 2B, which do not contain Arg-HCl. The viscosity for the formulation without Arg-HCl (1B) was observed to be higher than the viscocity of the formulation with Arg-HCl (1A) at 150 mg/mL as shown in Table 42 and FIG. 18.

TABLE 42
Viscocity for Formulations 1A and 1B at 20° C.

| | |
|---|---|
| | Viscosity (cP) measured |
| Time | at 20° C. |

| Point | 1A | 1B |
|---|---|---|
| T0 | 7.72 | 13.68 |

T0: Time zero;
1A: Formulation 1A;
1B: Formulation 1B

### Size Exclusion Chromatography (SEC)

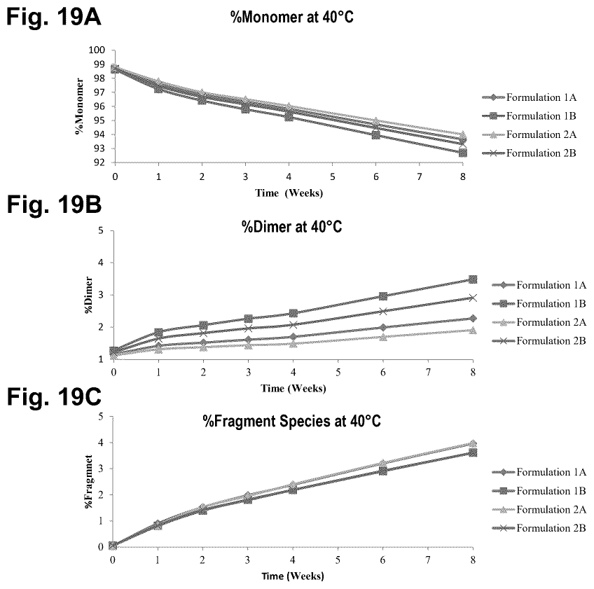

Tables 43-44 and FIGS. 19A-19C show the percent (%) monomer, % dimer and % fragment species of Formulations 1A-1B and 2A-2B measured by SEC after storage at 40° C. for up to 8 weeks. No significant difference in % monomer or % fragment was observed across the 4 formulations, as the reported values are within the method variability. Formulations 1B and 2B, which do not contain Arg-HCl, showed an increased rate of dimer formation compared to formulations 1A and 2A, which contain Arg-HCl, indicating a stabilizing effect of Arg-HCl on the drug product.

TABLE 43
% Monomer, % Dimer and % Fragment Species
for Formulations 1A-1B at 40° C.

| | Formulation 1A | | | Formulation 1B | | |
|---|---|---|---|---|---|---|
| | | | % | | | % |
| Time | % | % | Frag- | % | % | Frag- |
| Point | Monomer | Dimer | ment | Monomer | Dimer | ment |
| T0 | 98.8 | 1.2 | 0.0 | 98.6 | 1.3 | 0.0 |
| 1 WK | 97.6 | 1.4 | 0.9 | 97.2 | 1.9 | 0.8 |
| 2 WK | 96.9 | 1.5 | 1.4 | 96.4 | 2.1 | 1.4 |
| 3 WK | 96.3 | 1.6 | 1.9 | 95.8 | 2.3 | 1.8 |
| 4 WK | 95.8 | 1.7 | 2.4 | 95.2 | 2.4 | 2.2 |
| 6 WK | 94.7 | 2.0 | 3.2 | 93.9 | 2.9 | 2.9 |
| 8 WK | 93.7 | 2.3 | 3.9 | 92.7 | 3.5 | 3.6 |

T0: Time zero;
WK: Week(s)

TABLE 44
% Monomer, % Dimer and % Fragment Species
for Formulations 2A-2B at 40° C.

| | Formulation 2A | | | Formulation 2B | | |
|---|---|---|---|---|---|---|
| | | | % | | | % |
| Time | % | % | Frag- | Monomer | % | Frag- |
| Point | Monomer | Dimer | ment | % | Dimer | ment |
| T0 | 98.8 | 1.1 | 0.0 | 98.7 | 1.2 | 0.0 |
| 1 WK | 97.8 | 1.3 | 0.8 | 97.4 | 1.6 | 0.8 |
| 2 WK | 97.0 | 1.4 | 1.5 | 96.7 | 1.8 | 1.4 |
| 3 WK | 96.5 | 1.4 | 1.9 | 96.1 | 1.9 | 1.8 |
| 4 WK | 96.0 | 1.5 | 2.4 | 95.6 | 2.1 | 2.2 |
| 6 WK | 95.0 | 1.7 | 3.2 | 94.5 | 2.5 | 2.9 |
| 8 WK | 94.0 | 1.9 | 3.9 | 93.3 | 2.9 | 3.6 |

T0: Time zero;
WK: Week(s)

### Capillary Gel Electrophoresis (Reducing and Non-Reducing)

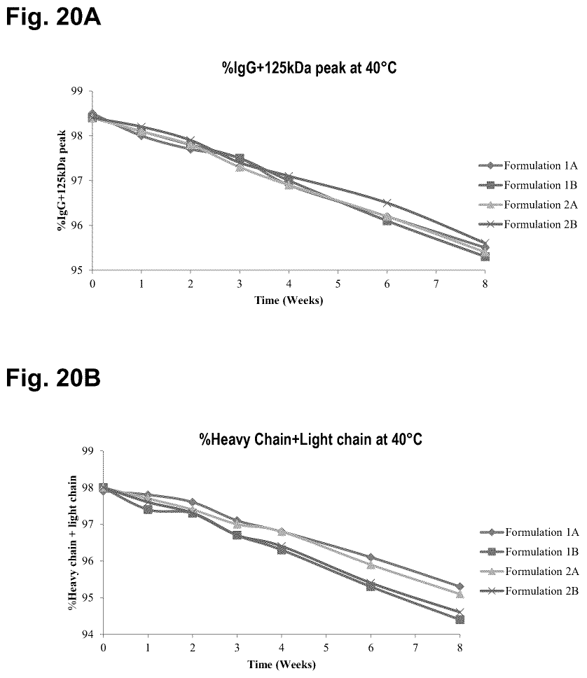

The percent (%) immunoglobulin G (IgG)+125 kDa peak was determined for Formulations 1A-1B and 2A-2B using non-reducing capillary gel electrophoresis (CGE) at the stressed condition of 40° C. for up to 8 weeks. In addition, the % heavy chain+light chain was determined for Formulations 1A-1B and 2A-2B using reducing CGE at the stressed condition of 40° C. for up to 8 weeks. These results are shown in Tables 45-46 and FIGS. 20A-20B, respectively. Formulations 1B and 2B, which lacked Arg-HCl, were observed to have increased rate of fragmentation compared to Formulations 1A and 2A, which contained Arg-HCl, demonstrating the stabilized effect of Arg-HCl on fragmentation pattern of the drug product.

TABLE 45
% IgG + 125 kDa Peak Measured Using Non-Reducing CGE
for Formulations 1A-1B and 2A-2B at 40° C.

| Time | Formulation | Formulation | Formulation | Formulation |
|---|---|---|---|---|
| Point | 1A | 1B | 2A | 2B |
| T0 | 98.5 | 98.4 | 98.4 | 98.4 |
| 1 WK | 98.0 | 98.1 | 98.1 | 98.2 |
| 2 WK | 97.7 | 97.8 | 97.8 | 97.9 |
| 3 WK | 97.5 | 97.5 | 97.3 | 97.4 |
| 4 WK | 96.9 | 97.0 | 96.9 | 97.1 |
| 6 WK | 96.2 | 96.1 | 96.2 | 96.5 |
| 8 WK | 95.5 | 95.3 | 95.4 | 95.6 |

T0: Time zero;
WK: Week(s)

TABLE 46
% Heavy Chain and Light Chain Measured Using Reducing
CGE for Formulations 1A-1B and 2A-2B at 40° C.

| Time | Formulation | Formulation | Formulation | Formulation |
|---|---|---|---|---|
| Point | 1A | 1B | 2A | 2B |
| T0 | 97.9 | 98.0 | 98.0 | 98.0 |
| 1 WK | 97.8 | 97.4 | 97.7 | 97.6 |
| 2 WK | 97.6 | 97.3 | 97.4 | 97.3 |
| 3 WK | 97.1 | 96.7 | 97.0 | 96.7 |
| 4 WK | 96.8 | 96.3 | 96.8 | 96.4 |
| 6 WK | 96.1 | 95.3 | 95.9 | 95.4 |
| 8 WK | 95.3 | 94.4 | 95.1 | 94.6 |

T0: Time zero;
WK: Week(s)

Capillary Isoelectric Focusing (icIEF)

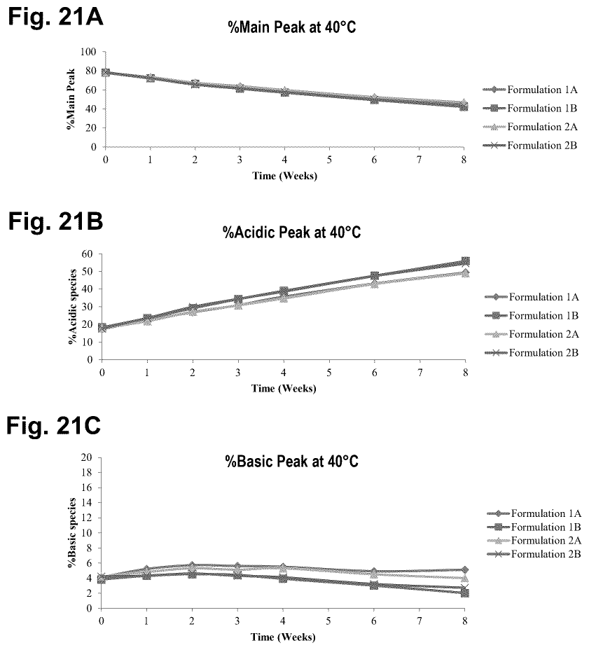

The percent (%) content of main species (main peak), % acidic species (acidic peak), and % basic species (basic peak) of Formulations 1A-1B and 2A-2B were measured using capillary isoelectric focusing (icIEF) at the stressed condition of 40° C. for up to 8 weeks. The results are shown in Tables 47-48. An overlay of stability trends in charge heterogeneity is presented in FIGS. 21A-21C. Slightly higher levels of acidic species were observed at the end of the 8 weeks for formulations 1B and 2B, which lacked Arg-HCl. This may be related to the higher rates of fragmentation evident for these formulations compared to formulations 1A and 2A, as reported in Table 46.

TABLE 47
% Main Peak, % Acidic Species and % Basic Species Determined
Using icIEF for Formulations 1A and 1B at 40° C.

| | Formulation 1A at 40° C. | | | Formulation 1B at 40° C. | | |
|---|---|---|---|---|---|---|
| Time | % Main | % Acidic | % Basic | % Main | % Acidic | % Basic |
| Point | peak | species | species | peak | species | species |
| T0 | 78.6 | 17.4 | 4.1 | 77.9 | 18.3 | 3.8 |
| 1 WK | 72.3 | 22.4 | 5.2 | 72.1 | 23.5 | 4.3 |
| 2 WK | 67.4 | 26.9 | 5.7 | 66.4 | 29.1 | 4.5 |
| 3 WK | 63.5 | 30.9 | 5.6 | 61.4 | 34.3 | 4.4 |
| 4 WK | 58.7 | 35.7 | 5.5 | 57.1 | 39.0 | 3.9 |
| 6 WK | 51.8 | 43.2 | 4.9 | 49.4 | 47.6 | 3.0 |
| 8 WK | 45.6 | 49.4 | 5.1 | 42.1 | 55.9 | 2.0 |

T0: Time zero;
WK: Week(s)

TABLE 48
% Main Peak, % Acidic Species and % Basic Species Determined
Using icIEF for Formulations 2A and 2B at 40° C.

| | Formulation 2A at 40° C. | | | Formulation 2B at 40° C. | | |
|---|---|---|---|---|---|---|
| Time | % Main | % Acidic | % Basic | % Main | % Acidic | % Basic |
| Point | peak | species | species | peak | species | species |
| T0 | 78.0 | 17.8 | 4.2 | 78.2 | 17.6 | 4.2 |
| 1 WK | 73.5 | 21.8 | 4.8 | 72.1 | 23.6 | 4.3 |
| 2 WK | 67.6 | 27.1 | 67.6 | 65.5 | 29.9 | 4.6 |
| 3 WK | 64.1 | 30.8 | 5.1 | 61.3 | 34.4 | 4.3 |
| 4 WK | 60.0 | 34.7 | 5.3 | 57.3 | 38.6 | 4.1 |
| 6 WK | 52.6 | 42.9 | 4.5 | 49.4 | 47.4 | 3.2 |
| 8 WK | 47.1 | 48.9 | 4.0 | 42.8 | 54.5 | 2.7 |

T0: Time zero;
WK: Week(s)

### Chemical Modifications to Primary Structure Using Peptide Mapping

Amino acids that could potentially undergo chemical modification over time and influence protein structure were monitored for Formulations 1A-1B and 2A-2B at the stressed condition of 40° C. for up to 8 weeks. Specifically, amino acid residues methionine 81 and methionine 254 of TEV-48574 could potentially undergo oxidation, affecting the primary structure, and were monitored. Similarly, asparagine 317 of TEV-48574 could potentially undergo deamidation, resulting in succinimide, and was monitored. No significant changes were observed in % Met81 oxidation, % Asn317 deamidation or % Met254 oxidation over time at the stressed storage condition of 40° C. for up to 8 weeks (data not shown), indicating that the presence of Arg-HCl does not appear to have an impact on this attribute.

### Potency by ELISA

The percent (%) potency of Formulations 1A-1B and 2A-2B was determined by enzyme-linked immunosorbent assay (ELISA) at the stressed storage condition of 40° C. for up to 8 weeks. The results are shown in Table 49. The trend of % potency was comparable across the formulations regardless of drug product concentration or the inclusion of Arg-HCl.

TABLE 49
% Relative Potency of Formulations 1A-1B and 2A-2B by ELISA

| | |
|---|---|
| | % Potency at 40° C. Acceptance |
| Time | criteria: 70-135% |

| Point | 1A | 1B | 2A | 2B |
|---|---|---|---|---|
| T0 | 107% | 103% | 105% | 106% |
| 2 WK | 100% | 94% | 98% | 98% |
| 4 WK | 86% | 84% | 88% | 87% |
| 8 WK | 89% | 89% | 85% | 86% |

T0: Time zero;
WK: Weeks;
1A: Formulation 1A;
1B: Formulation 1B;
2A: Formulation 2A;
2B: Formulation 2B

### Dynamic Light Scattering (DLS) for Particle Size Characterization

DLS was employed to measure the % polydispersity (% PD) and the hydrodynamic radius of the drug product and nanoparticles that might be present in Formulations 1A-1B and 2A-2B. DLS analysis was performed for a time zero (T0) sample of Formulations 1A-1B and 2A-2B. Table 50 shows the results of the DLS analysis. It was observed that the apparent hydrodynamic radius for formulations 1B and 2B (without Arg-HCl) was smaller compared to formulations 1A and 2A (with Arg-HCl).

TABLE 50
% Polydispersity (% PD) and Radius of Particles
for Formulations 1A-1B and 2A-2B

| | Formulation | | Formulation | | Formulation | | Formulation | |
|---|---|---|---|---|---|---|---|---|
| | 1A | | 1B | | 2A | | 2B | |
| Time | Radius | % | Radius | % | Radius | % | Radius | % |
| Point | (nm) | PD | (nm) | PD | (nm) | PD | (nm) | PD |
| T0 | 8.9 | 7.1 | 6.5 | 10.8 | 8.2 | 4.0 | 5.1 | 7.5 |

T0: Time zero

### Differential Scanning Calorimetry (DSC) Analysis for Thermal Stability

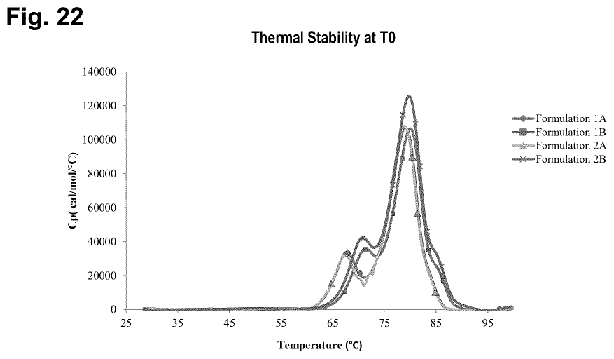

DSC was employed to evaluate thermal stability of Formulations 1A-1B and 2A-2B. DSC analysis was performed for a time zero (T0) sample of Formulations 1A-1B and 2A-2B. FIG. 22 shows the thermal stability of Formulations 1A-1B and 2A-2B at time zero (T0) and Table 51 shows the DSC thermogram for all 4 formulations. The Tonset was about 60° C. for formulations 1A and 2A, and about 63° C. for formulations 1B and 2B. The transition temperature (Tm) was about 78° C. for all formulations.

TABLE 51
DSC Thermogram for Formulations 1A-1B and 2A-2B

| Formulation | Tm1 (° C.) | Tm2 (° C.) |
|---|---|---|
| 1A | 67.9 | 78.8 |
| 1B | 71.2 | 79.8 |
| 2A | 67.5 | 78.6 |
| 2B | 70.7 | 79.5 |

The Tm1 for Formulations 1A and 2A was found to be slightly shifted to lower temperatures, demonstrating that Arg-HCl may impact the thermal stability of the molecule. This could be due to protein fragmentation observed in the samples at these conditions, leading to a decrease in enthalpy. However, this temperature is above the 2-8° C. recommended storage condition for this drug product.

### Conclusions

The effectiveness of Arginine-HCl (Arg-HCl) as an excipient and its impact on TEV-48574 drug product stability was assessed across two different drug product concentrations (100 mg/mL and 150 mg/mL). For all 4 formulations tested, the quality attributes of % dimer and % purity measured using reduced CGE indicated stabilizing effects of 100 mM Arg-HCl compared to formulations without Arg-HCl. Viscocity of the drug product at 150 mg/mL with Arg-HCl was lower compared to the formulation without Arg-HCl. A small shift in the Tm1 analyzed by DSC for the formulations containing Arg-HCl of about 3° C. was observed. However, the recommended storage condition for the drug product is 2-8° C., so this observed shift in Tm1 is not expected to significantly impact stability during typical storage and handling conditions. All other attributes did not show significant change over 8 weeks at 40° C. across all 4 formulations.

Based on these data, the 100 mM Arg-HCl in the TEV-48574 formulation has demonstrated the potential to stabilize the formulation and minimize viscocity as the concentration of the drug product is increased.

### Example 7: Pharmacokinetic and Pharmacodynamic Analysis in Non-Human Primates (NHP) in Response to TEV-48574

The objective of this study was to evaluate the pharmacokinetic (PK) properties of TEV-48574 in cynomolgus monkeys following a single subcutaneous (sc) administration of TEV-48574 at a dose of 5 mg/kg. Pharmacodynamic (PD) analysis was also performed for free and total TL1A in serum samples. In this study, 4 cynomolgus monkeys were treated with a single subcutaneous dose of TEV-48574 at a dose level of 5 mg/kg. Blood sampling was performed at Day 0 (pre-dose) and Days 0.25, 1, 2, 3, 4, 5, 7, 9, 11, 14, 21, 28 and 35 and analyzed for PK (TEV-48574) and PD (free and total TL1A).

For the PK assessment, serum samples were analyzed using an enzyme linked immunosorbent assay (ELISA) method to determine TEV-48574 concentrations. PK parameters for TEV-48574 were estimated from the individual serum concentration-versus-time profiles by noncompartmental analysis (Gibaldi, M., & Perrier, D. (Eds.) (1982). Pharmacokinetics (2nd ed.) CRC Press). Serum concentrations below the limit of quantitation (ie, <100 ng/mL) were designated as “BLQ”. For purposes of calculating the mean concentrations, all BLQ values were treated as zero.

The maximum serum concentration (Cₘₐₓ) was the highest observed serum concentration. The tₘₐₓ was the corresponding time when Cₘₐₓ was observed. The terminal rate constant for elimination from serum (λ_{z}) was determined by linear regression of the terminal portion of the respective semi-logarithmic serum concentration-versus-time curve. The terminal phase was identified for each profile by visual inspection of the data, with a minimum of 3 non-BLQ concentrations included for each λ_{z} determination. The terminal half-life (t_{1/2}) was calculated as ln(2) divided by λ_{z}. The λ_{z} and related parameters were considered reliable if r²-adjusted was ≥0.8 and the time interval over which λ_{z} was estimated was approximately as long as or longer than the corresponding λ_{z}. The area under the serum concentration-versus-time curves from time zero to the time of the last measurable concentration (AUC₀₋ₜ) was determined by linear trapezoidal summation. The area under the serum concentration-versus-time curve from time zero to infinity (AUC_{0-∞}) was calculated as the sum of AUC₀₋ₜ and the area extrapolated from the time of the last measurable serum concentration to infinity (Cₗₐₛₜ/λ_{z}) and was considered reliable if the percentage of AUC_{0-∞} extrapolated [(AUC_{0-∞}−AUC₀₋ₜ)/(AUC_{0-∞})×100] was <20%. PK parameters were estimated using the nominal sample collection times.

Concentrations of TEV-48574 were BLQ in all predose samples and quantifiable in all animals at the 1^{st} postdose sampling point (i.e., at 6 hours). The intra-animal variability generally was low at the early timepoints then increased starting on day 11. In all animals, the serum concentrations were fairly constant starting on day 1 or 2 through day 9, 11, or 21; the coefficient of variations (CVs) of the concentrations in these timeframes were 6.1%, 8.7%, 6.4%, and 13.7% for animals SC1607075 (days 2-11), SC1511041 (days 1-9), SC1607037 (days 1-9), and SC1506091 (days 1-21), respectively, and mainly reflect absorption and distribution. Thereafter, concentrations declined sharply and, with the exception of monkey SC1506091, were BLQ starting on day 14 or 21. Analysis for monkey anti-TEV-48574 antibodies by ELISA confirmed the presence of inducible ADA in the day 14, day 21, and day 35 serum samples from all animals.

The serum TEV-48574 concentration-time profiles for each animal were analyzed by noncompartmental analysis (NCA), and the PK parameters are presented in Table 52. Absorption following SC administration was consistent across animals. Tₘₐₓ ranged from 3 to 5 days (median=4 days), and the Cₘₐₓ values were similar in the 4 monkeys (CV=18.4%). The ty and AUC values, however, were variable. Target mediated clearance of TEV-48574 was not evident in this study.

TABLE 52
Pharmacokinetic Parameters of TEV-48574 for Cynomolgus Monkeys
Administered Single 5-mg/kg SC Doses of TEV-48574

| | | Animal ID | | | | | | |
|---|---|---|---|---|---|---|---|---|
| Parameter | Units | SC1506091 | SC1511041 | SC1607037 | SC1607075 | Mean | SD | CV |
| R²-adjusted | — | 0.996 | 0.579 | 0.987 | 0.829 | — | — | — |
| Span | — | 3.134 | 3.223 | 3.552 | 1.047 | — | — | — |
| t_{1/2} | (day) | 4.5 | 1.2^{a} | 1.4 | 4.8 | 3.0 | 1.9 | 64.2 |
| tₘₐₓ | (day) | 5 | 4 | 4 | 3 | 4^{b} | — | — |
| Cₘₐₓ | (μg/mL) | 13.13 | 10.95 | 8.99 | 9.14 | 10.55 | 1.94 | 18.4 |
| AUC₀₋ₜ | (μg · day/mL) | 254 | 92 | 88 | 109 | 136 | 79 | 58.4 |
| AUC_{0-∞} | (μg · day/mL) | 261 | 94^{a} | 90 | 140^{c} | 146 | 80 | 54.5 |
| AUC Extrap | (%) | 2.5 | 1.9 | 1.6 | 22.3 | — | — | — |

^{a}R²-adjusted <0.8;
^{b}Median;
^{c}% Extrapolated >20%

NHP serum samples were tested for the presence of total TL1A (free and bound to TEV-48574) using a 96-well plate immunoassay with electrochemiluminescence (ECL) detection. Briefly, each 96-well small-spot streptavidin sector plate was blocked (to reduce non-specific binding) with Diluent A (casein in phosphate-buffered saline (PBS)). The plate was incubated with 500 rpm shaking at a temperature range of 20°−23° C. for 60 minutes. Following the blocking step, the wells were aspirated and then coated with a 2 μg/mL stock of biotinylated rabbit anti-TL1A polyclonal antibody diluted into Diluent A. The capture antibody coating proceeded for 60 minutes with 500 rpm shaking at a temperature range of 20°−23° C. The wells in the 96-well plate were then washed with Wash Buffer (PBS containing 0.05% Tween-20). After washing, Diluent A was added to each well. To serve as assay standards, a 5000 μg/mL recombinant cynomolgus monkey TL1A protein stock along with six subsequent 1:4 dilutions were prepared in Diluent A. To designated assay wells, either one of the recombinant monkey TL1A protein standards or a 1:4 dilution of NHP serum in Diluent A were added. The assay plate was then incubated with 500 rpm shaking for 1 hour at a temperature range of 20°−23° C.

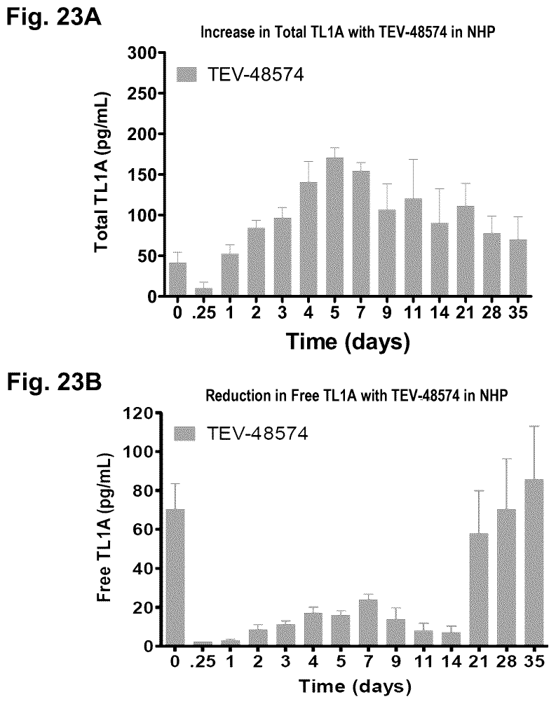

After incubation with standards and serum samples, the wells were washed with Wash Buffer. In order to saturate any free TL1A captured in the wells, a 2 μg/mL stock of unconjugated TEV-48574 diluted into Diluent A was added. Following a 60-minute incubation with 500 rpm shaking at a temperature range of 20°-23° C., the wells were once again washed. For detection, a 1 μg/mL stock of sulfo-tagged goat anti-human IgG polyclonal antibody diluted into Diluent A was added to the assay wells. The detection antibody was incubated protected from light with 500 rpm shaking for 1 hour at a temperature range of 20°−23° C. After incubation with the sulfo-tagged goat anti-human IgG polyclonal antibody, the wells were once again washed with Wash Buffer. Read Buffer was added to the assay wells, and the ECL signal was measured using a Meso Scale Discovery (MSD) sector imager 600. Calculated total TL1A concentration in NHP serum samples was then determined. The increase in total TL1A in this NHP study (FIG. 23A) was found to be modest, consistent with results obtained in a first in human study.

To determine free TL1A levels (unbound to TEV-48574) in NHP serum samples (i.e., the same NHP serum samples used to determine total TL1A levels), a 96-well plate immunoassay with ECL detection was performed. Briefly, each 96-well plate was blocked with Diluent A. The plate was incubated with 500 rpm shaking at a temperature range of 20°−23° C. for 60 minutes. Following the blocking step, the wells were aspirated and then coated with a 2 μg/mL stock of biotinylated rabbit anti-TL1A polyclonal antibody diluted into Diluent A. The capture antibody coating proceeded for 60 minutes with 500 rpm shaking at a temperature range of 20°-23° C. The wells in the 96-well plate were then washed with Wash Buffer. After washing, Diluent A was added to each well. To serve as assay standards, a 5000 μg/mL recombinant cynomolgus monkey TL1A protein stock and six subsequent 1:4 dilutions were prepared in Diluent A. To designated assay wells, either one of the recombinant monkey TL1A protein standards or a 1:4 dilution of NHP serum in Diluent A was added. The assay plate was then incubated with 500 rpm shaking for 1 hour at a temperature range of 20°-23° C. After incubation with standards and serum samples, the wells were washed with Wash Buffer. For detection, a 1 μg/mL stock of sulfo-tagged TEV-48574 diluted into Diluent A was added to the assay wells. The detection antibody was incubated (protected from light) with 500 rpm shaking for 1 hour at a temperature range of 20°-23° C. After incubation with the sulfo-tagged TEV-48574, the wells were washed with Wash Buffer. Read Buffer was added to the assay wells. The ECL signal was measured using an MSD sector imager 600. Calculated free TL1A concentration in NHP serum samples was then determined.

In contrast to the total TL1A levels measured in the NHP serum samples, the free TL1A levels were found to be suppressed in all animals well below the pre-dosing baseline for at least 14 days. The pre-dosing baseline TL1A levels ranged from 43 to 106 μg/mL, dropping to below the limit of quantitation at the earliest time point (6 hours) post-dose. From Days 2 to 14, the levels remained low at only 10% to 40% of the baseline. Levels had returned to baseline by Day 21. The duration of suppression observed may be an underestimate due to the anti-drug antibody response observed from Day 14 (FIG. 23B).

This NHP data provides evidence that unlike other monoclonal antibodies directed against TL1A, effective target engagement by TEV-48574 as measured by free TL1A is not reflected as a large increase in total TL1A. Thus, TEV-48574 resulted in a profound and sustained suppression of free TL1A. The PD effect reflected in the suppression of free TL1A levels, has a longer effect than that predicted by the half-life (T_{1/2}=3.0+/−1.9 days), suggesting that a dosing interval of every 4 weeks could also be used.

### Example 8: Population Pharmacokinetic (PPK) Modeling and Simulation of TEV-48574 Dosing Regimens

A comparison of exposure between TEV-48574 and RVT-3101 (previously known as PF-06480605, and described in PCT Publication Nos. WO 2015/073580 A1 and WO 2021/260577, each of which is incorporated herein by reference) was performed using population pharmacokinetic (PPK) modelling and simulation approaches. These calculations were aimed to describe the time-course of serum TEV-48574 concentrations in healthy and asthmatic participants obtained from three clinical studies involving TEV-48574, including single ascending dose (SAD) data from healthy participants in the TV48574-SAD-10126 study, multiple ascending dose (MAD) data from asthma patients in the TV48574-SAD-10126 study, and single dose data from healthy participants (Japanese and Caucasian).

### Data Selection and Study Design(s)

For each of the following studies, one or more of following pharmacokinetic parameters was assessed: maximum observed serum drug concentration (Cₘₐₓ); time to maximum observed serum drug concentration (tₘₐₓ); area under the serum concentration-time curve from time 0 to the time of the last measurable TEV-48574 concentration (AUC₀₋ₜ); area under the serum concentration-time curve extrapolated to infinity (AUC_{0-∞}); percentage of the extrapolated area to infinity in relation to the total area under the curve (% AUCₑₓₜ); apparent serum terminal elimination rate constant (λ_{z}) and associated terminal phase (apparent elimination) half-life (t_{1/2}); apparent total body clearance (CL/F); and apparent volume of distribution during terminal phase (V_{z}/F).

Single Ascending Dose (SAD) Data from Healthy Participants in TV48574-SAD-10126 Study

The SAD part of Study TV48574-SAD-10126 was a single-center, randomized, double-blind, placebo-controlled, parallel-group Phase 1 study to evaluate the safety, tolerability, PK, and immunogenicity of TEV-48574 in approximately 64 healthy adult men and women 18 to 60 years of age. The SAD part consisted of a screening period, ambulatory visits, ADA assessments, an End-of-Treatment (EOT) visit on Day 22, and an End-of-Study visit on Day 57.

On Day 1, subjects were administered a SC injection of placebo or 1, 4, 12, 36, 90, 200, 400, or 1000 mg TEV-48574.

Pharmacokinetic parameters were based on the pharmacokinetic analysis set, which consisted of those subjects in the safety analysis set who had at least 1 measurable concentration of TEV-48574. All 48 subjects who received TEV-48574 in the SAD portion of the study qualified for the pharmacokinetic analysis set.

Following single-dose SC administration of 1 mg and 4 mg TEV-48574, all serum TEV-48574 concentrations were BLQ (<200 ng/ml). After administration of 12 mg, 36 mg, 90 mg, 200 mg, 400 mg, and 1000 mg TEV-48574, serum TEV-48574 concentrations increased with increasing dose. TEV-48574 serum concentrations were quantifiable 6 hours post-dose for the 12 mg, 36 mg, and 90 mg doses and at the first time point (1 hour post-dose) for the 200 mg, 400 mg, and 1000 mg dose levels. TEV-48574 peak serum concentrations were typically reached between 72 and 96 hours post-dose. After reaching peak concentrations, TEV-48574 was eliminated gradually in a monophasic manner. The rate of elimination appeared similar across dose levels.

TEV-48574 serum concentrations were quantifiable until 288 hours (12 days) in the 12 mg cohort; until 672 hours (28 days) in the 36 mg and 90 mg cohorts; and until 1344 hours (56 days) in the 200 mg (n=1), 400 mg (n=4), and 1000 mg (n=4) cohorts.

Following single-dose SC administrations of TEV-48574, median tₘₐₓ of TEV-48574 in serum occurred at 96 hours for the 12 mg, 36 mg, and 90 mg dose cohorts and 72 hours for the 200 mg, 400 mg, and 1000 mg dose cohorts.

Geometric mean peak serum concentrations (Cₘₐₓ) and total systemic exposures (AUC₀₋ₜ and AUC_{0-∞}) increased with an increase in dose from 12 mg to 1000 mg. The increase in peak and total systemic exposure from 36 mg to 1000 mg appeared to be slightly greater than dose proportional. The increase in peak and total systemic exposure from 200 mg to 1000 mg appeared to be dose proportional.

Mean t_{1/2} estimates ranged from 155.85 to 230.88 hours (ie, approximately 6.5 to 9.6 days). Mean CL/F and V_{z}/F estimates ranged from 0.66 to 1.71 L/day for CL/F and 8.24 to 15.13 L for V_{z}/F.

The variability (CV %) of systemic exposure parameters ranged from 23.57% to 53.43% for Cₘₐₓ and from 17.03% to 61.27% for the AUCs (AUC₀₋ₜ and AUC_{0-∞}) across all dose levels, as shown in Table 53.

TABLE 53
Summary of TEV-48574 Serum Pharmacokinetic Parameters by SAD Cohort

| | | Cohort 3 | Cohort 4 | Cohort 5 | Cohort 6 | Cohort 7 | Cohort 8 |
|---|---|---|---|---|---|---|---|
| | | 12 mg | 36 mg | 90 mg | 200 mg | 400 mg | 1000 mg |
| Parameter | | TEV-48574 | TEV-48574 | TEV-48574 | TEV-48574 | TEV-48574 | TEV-48574 |
| (unit) | Statistic | (N = 6)^{a} | (N = 6) | (N = 6) | (N = 6) | (N = 6) | (N = 6) |
| Cₘₐₓ (ng/mL) | n | 6 | 6 | 6 | 6 | 6 | 6 |
| | Mean | 447 | 1633.83 | 4581.33 | 15007.17 | 41284.33 | 74508.33 |
| | SD | 238.847 | 741.977 | 1389.902 | 3536.574 | 10523.843 | 26145.471 |
| | % CV | 53.43 | 45.41 | 30.34 | 23.57 | 25.49 | 35.09 |
| | Median | 494.5 | 1898.5 | 4705 | 16258 | 38421 | 79032 |
| | Geometric | 528.12 | 1454.89 | 4395.74 | 14623.11 | 40206.08 | 69292.8 |
| | Mean | | | | | | |
| | Min, max | 0, | 628, | 3001, | 10022, | 30475, | 28672, |
| | | 689 | 2471 | 6111 | 18319 | 55303 | 107546 |
| tₘₐₓ (h) | n | 5 | 6 | 6 | 6 | 6 | 6 |
| | Median | 96 | 96 | 96 | 72.06 | 72.02 | 72.05 |
| | Min, max | 72, | 96, | 48, | 48, | 48, | 72, |
| | | 120 | 144 | 120 | 120 | 120 | 96.1 |
| AUC₀₋ₜ | n | 6 | 6 | 6 | 6 | 6 | 6 |
| (h*ng/mL) | | | | | | | |
| | Mean | 91821.18 | 474514.86 | 1174928.12 | 4464500.17 | 14652979.88 | 27868516.36 |
| | SD | 47278.078 | 290742.669 | 397954.719 | 767695.593 | 3200860.317 | 13403532.634 |
| | % CV | 51.49 | 61.27 | 33.87 | 17.2 | 21.84 | 48.1 |
| | Median | 105456.95 | 512668.63 | 1164565.94 | 4659322.47 | 13357745.72 | 26957652.22 |
| | Geometric | 109239.17 | 375989.4 | 1116316.63 | 4405647.29 | 14394603.48 | 24511826.51 |
| | Mean | | | | | | |
| | Min, max | 0, | 133382.2, | 670282.2, | 3283128.5, | 11788689.4, | 8442470.9, |
| | | 133788.7 | 808575.6 | 1713190.3 | 5291845.6 | 20508396.6 | 44398899.1 |
| AUC_{0-∞} | n | — | 4 | 4 | 6 | 6 | 6 |
| (h*ng/mL) | | | | | | | |
| | Mean | — | 730919.52 | 1382212.87 | 4565624.17 | 14977092.14 | 28917545.57 |
| | SD | — | 196904.082 | 467018.723 | 777683.112 | 3356321.535 | 14485706.661 |
| | % CV | — | 26.94 | 33.79 | 17.03 | 22.41 | 50.09 |
| | Median | — | 715879.37 | 1366752.36 | 4739879.7 | 13561047.33 | 27482919.13 |
| | Geometric | — | 711056.7 | 1321531.55 | 4506465.46 | 14702227.05 | 25225765.43 |
| | Mean | | | | | | |
| | Min, max | — | 529509.6, | 883722.9, | 3352166.5, | 12155271.6, | 8586278.7, |
| | | | 962409.8 | 1911623.9 | 5396707.7 | 21189585.5 | 47197439.7 |
| % AUCextrap | n | — | 4 | 4 | 6 | 6 | 6 |
| | Mean | — | 11.35 | 7.04 | 2.24 | 2.09 | 2.81 |
| | SD | — | 3.128 | 2.366 | 0.66 | 0.804 | 2.332 |
| | % CV | — | 27.56 | 33.62 | 29.54 | 38.46 | 83.07 |
| | Median | — | 10.16 | 6.37 | 2 | 1.65 | 2.31 |
| | Geometric | — | 11.07 | 6.77 | 2.16 | 1.97 | 1.79 |
| | Mean | | | | | | |
| | Min, max | — | 9.1, | 5, | 1.6, | 1.5, | 0.3, |
| | | | 16 | 10.4 | 3.4 | 3.2 | 5.9 |
| t½ (h) | n | — | 4 | 4 | 6 | 6 | 6 |
| | Mean | — | 182.96 | 155.85 | 179.43 | 209.22 | 230.88 |
| | SD | — | 47.257 | 35.719 | 32.439 | 31.875 | 88.906 |
| | Median | — | 169.49 | 154.1 | 174.1 | 207.66 | 209.16 |
| | Min, max | — | 143.2, | 114.4, | 131.3, | 170.4, | 147, |
| | | | 249.7 | 200.8 | 229.7 | 261.6 | 339.3 |
| CL/F (L/day) | n | — | 4 | 4 | 6 | 6 | 6 |
| | Mean | — | 1.25 | 1.71 | 1.08 | 0.66 | 1.14 |
| | SD | — | 0.334 | 0.596 | 0.205 | 0.123 | 0.852 |
| | Median | — | 1.23 | 1.64 | 1.02 | 0.71 | 0.87 |
| | Min, max | — | 0.9, | 1.1, | 0.9, | 0.5, | 0.5, |
| | | | 1.6 | 2.4 | 1.4 | 0.8 | 2.8 |
| Vz/F (L) | n | — | 4 | 4 | 6 | 6 | 6 |
| | Mean | — | 13.1 | 15.13 | 11.56 | 8.24 | 13.46 |
| | SD | — | 1.102 | 2.174 | 2.607 | 1.367 | 6.808 |
| | Median | — | 13.42 | 15.22 | 10.47 | 8.81 | 11.16 |
| | Min, max | — | 11.5, | 12.9, | 9.4, | 6, | 7.9, |
| | | | 14 | 17.2 | 15 | 9.5 | 26.9 |

Per the SAP, λ_{z} parameters were not reported if percent extrapolation was >20% and/or the interval over which λ_{z} was estimated was shorter than the corresponding t_{1/2}.
BLQ = below the limit of quantitation; max = maximum; Min = minimum; N = total number of subjects; n = number of subjects with evaluable data; SAD = single ascending dose; SAP = statistical analysis plan; SD = standard deviation.
Note:
Serum pharmacokinetic parameters for cohort 1 (1 mg) and cohort 2 (4 mg) could not be calculated because all serum TEV-48574 concentrations were BLQ (<200 ng/mL).

Systemic absorption of SC TEV-48574 was slow in all cohorts. The median tₘₐₓ was 96 hours in the 12 mg, 36 mg, and 90 mg dose cohorts and 72 hours in the 200 mg, 400 mg, and 1000 mg dose cohorts. PK parameters after the 1 mg and 4 mg doses could not be calculated due to serum TEV-48574 bioassay limitations (all concentrations were BLQ of <200 ng/ml). Increased doses of TEV-48574 from 12 mg to 1000 mg resulted in increasing TEV-48574 peak (Cₘₐₓ) and total (AUCs) systemic exposure.

Assessing dose proportionality using TEV-48574 exposure parameters (Cₘₐₓ, AUC₀₋ₜ, and AUC_{0-∞}) within the power model indicated that the increase in exposure from 36 mg to 1000 mg was greater than dose proportional. Over the 200 mg to 1000 mg dose range, the increase in exposure was dose proportional. TEV-48574 was shown to have a half-life generally exceeding 6.5 days (range: 155.85 to 230.88 hours, ie, approximately 6.5 to 9.6 days) and was not impacted by dose level. The mean CL/F and V_{z}/F estimates ranged from 0.66 to 1.71 L/day for CL/F and 8.24 to 15.13 L for V_{z}/F.

In the SAD portion of this study, following single SC doses of TEV-48574 at 1 mg, 4 mg, 12 mg, 36 mg, 90 mg, 200 mg, 400 mg, and 1000 mg in healthy volunteers, the pharmacokinetics of TEV-48574 were characterized by slow absorption of drug into the systemic circulation. After 1 mg and 4 mg doses, all TEV-48574 serum concentrations were BLQ. TEV-48574 peak serum concentrations were typically reached between 72 and 96 hours post-dose. After reaching peak concentrations, TEV-48574 was eliminated gradually in a monophasic manner. The rate of elimination appeared similar across dose levels.

Across the dose range tested in the SAD portion, TEV-48574 peak (Cₘₐₓ) and total (AUC₀₋ₜ and AUC_{0-∞}) systemic exposure increased with increasing dose. From 36 mg to 1000 mg, the increase in TEV-48574 exposure was greater than dose proportional, indicated by the 90% CI for the estimated slope for Cₘₐₓ and the AUCs not including unity (1.0). Over the 200 mg to 1000 mg dose range, the increase in exposure was dose proportional.

The t_{1/2} of TEV-48574 in the SAD portion was similar across the studied dose range; TEV-48574 had a half-life generally exceeding 6.5 days in all SAD cohorts (range: 155.85 to 230.88 hours, ie, approximately 6.5 to 9.6 days). For CL/F, mean estimates were similar across the dose range. The V_{z}/F was also similar across the dose range studied in the SAD portion (8.24 to 15.13 L), indicating that TEV-48574 was mostly confined to the circulatory system.

Overall, the statistical analysis of dose proportionality after single doses in the SAD (36 to 1000 mg range) suggested slight supra-proportionality in TEV-48574 pharmacokinetics. However, TEV-48574 half-life, CL/F, and V_{z}/F appeared similar across all SAD doses, which is indicative of linear pharmacokinetics. Over the 200 mg to 1000 mg dose range, dose proportionality was observed. Therefore, the overall slightly greater than dose proportional results may be less relevant as only doses of 200 mg or greater were brought forward into the MAD portion.

Multiple Ascending Dose (MAD) Data from Asthma Patients in TV48574-SAD-10126 Study

The MAD part of Study TV48574-SAD-10126 was a randomized, double-blind, placebo-controlled, parallel group study to evaluate the safety, tolerability, PK and immunogenicity of TEV-48574 following three doses administered as a SC injection in approximately 36 male and female symptomatic asthma patients, 18 to 65 years of age. The MAD part consisted of a prescreening (optional) and screening period, a 14- to 28-day run-in/washout period, an in-house period, ambulatory visits, ADA assessments, EOT visit (Day 43), and an End-of-Study visit (Day 92).

Patients in the MAD portion of the study were assigned to 1 of 3 cohorts (N=12 per cohort) to receive SC doses of TEV-48574 or placebo Q2W on Days 1, 15, and 29 of the study. TEV-48574 was administered at doses of: (1) 200 mg Q2W as a single SC injection using standardized syringes; (2) 800 mg followed by 600 mg Q2W as a SC injection using standardized syringes; or (3) 2300 mg followed by 1600 mg as an approximately 1-hour SC infusion. In each cohort, 3 participants were randomly assigned TEV-48574 (N=2) or placebo (N=1) 72 hr prior to the other participants. Provided no stopping criteria had been met and no safety concerns related to TEV-48574 had been identified, at the discretion of the principal investigator, 72 hours after TEV-48574 administration to the first 3 patients, the remaining patients in the cohort were randomly assigned to receive TEV-48574 (7 patients) or placebo (2 patients). Patients could be dosed individually or in a cohort fashion at the discretion of the principal investigator.

Following the first dose SC administrations of TEV-48574 on day 1, TEV-48574 geometric mean Cₘₐₓ, AUC₀₋ₜ, and AUC, increased 8.10-, 8.26- and 8.60-fold with an 11.5-fold increase in dose from 200 mg to 2300 mg. Variability in exposure ranged from 42.3% to 83.7% for Cₘₐₓ, 38.0% to 77.3% for AUC₀₋ₜ and 40.8% to 92.6% for AUC_{τ} (geometric % CVs). TEV-48574 median tₘₐₓ in serum occurred at approximately 3 days (range: 2.97 to 2.99 days) for the 200 mg, 800 mg, and 2300 mg dose cohorts.

After multiple-dose SC administration, TEV-48574 geometric mean Cₘₐₓ, AUC₀₋ₜ and AUC_{τ} increased 6.85-, 7.93- and 5.24-fold, respectively, on day 15 and 6.38-, 9.26- and 5.35-fold, respectively, on day 29, with an 8-fold increase in dose from 200 mg to 1600 mg. Variability (geometric CV %) after multiple-dose exposure (day 15 and day 29) ranged from 31.1% to 78.2% for Cₘₐₓ, 24.6% to 74.2% for AUC₀₋ₜ and 23.0% to 74.1% for AUC_{τ} for the patients without treatment-emergent ADA. Overall, variability in exposure (Cₘₐₓ, AUC₀₋ₜ and AUC_{τ}) was moderate in all cohorts and similar after each administration of TEV-48574. Additionally, median tₘₐₓ after multiple-dose administration was similar to the first dose and ranged from 2.96 to 3.00 days across the dose range studied, as shown in Table 54.

TABLE 54
Summary of TEV-48574 Serum Pharmacokinetic Parameters by MAD Cohort

| | | | | Cohort 2: | Cohort 3: |
|---|---|---|---|---|---|
| | | | Cohort 1: | 800 mg (ld)/ | 2300 mg (ld)/ |
| | | | 200 mg | 600 mg (md) | 1600 mg (md) |
| | Parameter | | TEV-48574 | TEV-48574 | TEV-48574 |
| Day | (unit) | Statistic | (N = 9) | (N = 9) | (N = 9) |
| Day 1 | Cₘₐₓ (ng/mL) | n | 9 | 9 | 9 |
| | | Mean | 14068.7 | 45303.3 | 130840.4 |
| | | SD | 5509.26 | 19958.73 | 83001.05 |
| | | Median | 14378 | 44853 | 131742 |
| | | Geometric | 13109.9 | 40996.8 | 106215.6 |
| | | mean | | | |
| | | Geometric CV | 42.3 | 53.4 | 83.7 |
| | | Min, max | 6914, 24365 | 14956, 80916 | 33512, 296131 |
| Day 1 | tₘₐₓ (day) | n | 9 | 9 | 9 |
| | | Median | 2.975 | 2.971 | 2.988 |
| | | Min, max | 1.971, 3.988 | 1.992, 3.017 | 2.917, 5.958 |
| Day 1 | AUC₀₋ₜ | n | 9 | 9 | 9 |
| | (h*ng/mL) | Mean | 2875335.4 | 9528669.9 | 26898480.7 |
| | | SD | 1075805.25 | 4320346.61 | 16125950.63 |
| | | Median | 2843291 | 9411584 | 28703843 |
| | | Geometric | 2709188 | 8556319.8 | 22389577.8 |
| | | mean | | | |
| | | Geometric CV | 38 | 56.4 | 77.3 |
| | | Min, max | 1457018, 5238053 | 2909578, 17986118 | 7669479, 60805717 |
| Day 1 | AUC_{τ} (h*ng/mL) | n | 8 | 9 | 7 |
| | | Mean | 2883371.6 | 9537985.3 | 28946775.9 |
| | | SD | 1150272.12 | 4324269.87 | 18077602.86 |
| | | Median | 2805804.5 | 9432210 | 30893750 |
| | | Geometric | 2696690.6 | 8564178.7 | 23195366.4 |
| | | mean | | | |
| | | Geometric CV | 40.8 | 56.4 | 92.6 |
| | | Min, max | 1457162, 5239092 | 2909823, 17998881 | 7689248, 60890050 |
| Day 15 | Cₘₐₓ (ng/mL) | n | 9 | 9 | 9 |
| | | Mean | 19237.4 | 49391.2 | 133304.8 |
| | | SD | 8447.45 | 17707.93 | 68752.66 |
| | | Median | 18813 | 45481 | 146535 |
| | | Geometric | 16447.9 | 46856.9 | 112674 |
| | | mean | | | |
| | | Geometric CV | 81.7 | 34.8 | 78.2 |
| | | Min, max | 2857, 31594 | 29786, 81248 | 26593, 255216 |
| Day 15 | tₘₐₓ (day) | n | 9 | 9 | 9 |
| | | Median | 3 | 3 | 2.963 |
| | | Min, max | 2.021, 4.067 | 2, 3.946 | 2.042, 3.008 |
| Day 15 | AUC₀₋ₜ | n | 9 | 9 | 9 |
| | (h*ng/mL) | Mean | 3944229.1 | 9719685.3 | 29527453.4 |
| | | SD | 1701465.2 | 3673546.12 | 15308249.69 |
| | | Median | 3963546 | 8857805 | 30858163 |
| | | Geometric | 3174089.8 | 9191646.8 | 25165689.3 |
| | | mean | | | |
| | | Geometric CV | 117.6 | 35.5 | 74.2 |
| | | Min, max | 285130, 6172921 | 6126492, 17538029 | 6502594, 56980049 |
| Day 15 | AUC_{τ} (h*ng/mL) | n | 5 | 5 | 9 |
| | | Mean | 4907967.4 | 10662414.4 | 29555777.2 |
| | | SD | 1082520.99 | 4105076.69 | 15315099.56 |
| | | Median | 5091057 | 10048362 | 30878247 |
| | | Geometric | 4809693 | 10116395.8 | 25196813.6 |
| | | mean | | | |
| | | Geometric CV | 23 | 36.4 | 74.1 |
| | | Min, max | 3648938, 6178790 | 6635002, 17544613 | 6513731, 57044497 |
| Day 29 | Cₘₐₓ (ng/mL) | n | 8 | 7 | 9 |
| | | Mean | 19759.4 | 46203.4 | 125770.7 |
| | | SD | 9215.81 | 18229.73 | 62686.07 |
| | | Median | 18226 | 39405 | 131021 |
| | | Geometric | 17016.4 | 43613.1 | 108649.1 |
| | | mean | | | |
| | | Geometric CV | 75.9 | 36.5 | 68.6 |
| | | Min, max | 3819, 32431 | 29714, 80371 | 38409, 209540 |
| Day 29 | tₘₐₓ (day) | n | 8 | 7 | 9 |
| | | Median | 2.9815 | 2.975 | 2.971 |
| | | Min, max | 1.029, 3.967 | 2.963, 4.038 | 1.988, 5.958 |
| Day 29 | t_{1/2} (day) | n | 6 | 7 | 9 |
| | | Mean | 8.2987 | 8.631 | 8.713 |
| | | SD | 1.83366 | 2.6332 | 2.59108 |
| | | Median | 8.75 | 7.583 | 7.667 |
| | | Min, max | 4.917, 10 | 5.042, 12.417 | 6.208, 14.708 |
| Day 29 | AUC₀₋ₜ | n | 8 | 7 | 9 |
| | (h*ng/mL) | Mean | 7146681 | 19493831.1 | 48642292.7 |
| | | SD | 4584498.78 | 11691552.17 | 26722092.8 |
| | | Median | 7446594 | 15518284 | 43912333 |
| | | Geometric | 4557546.7 | 17045599.3 | 42200283.1 |
| | | mean | | | |
| | | Geometric CV | 246.5 | 58.5 | 63.3 |
| | | Min, max | 183049, 13684437 | 8553435, 41485341 | 17570630, 93618071 |
| Day 29 | AUC_{τ} (h*ng/mL) | n | 7 | 7 | 9 |
| | | Mean | 5129416.3 | 11216582.7 | 29562570.3 |
| | | SD | 1792625.49 | 5097448.27 | 14204656.79 |
| | | Median | 4572855 | 10007329 | 28837363 |
| | | Geometric | 4864042 | 10374452.5 | 26022895.5 |
| | | mean | | | |
| | | Geometric CV | 36.4 | 43.4 | 62.1 |
| | | Min, max | 3271961, 7555080 | 6063501, 20936911 | 10657178, 50957418 |
| Day 29 | CL/F (L/h) | n | 7 | 7 | 9 |
| | | Mean | 0.0433 | 0.062 | 0.0719 |
| | | SD | 0.01467 | 0.0233 | 0.04614 |
| | | Median | 0.044 | 0.06 | 0.055 |
| | | Min, max | 0.026, 0.061 | 0.029, 0.099 | 0.031, 0.15 |
| Day 29 | V_{z}/F (L) | n | 7 | 7 | 9 |
| | | Mean | 11.79 | 16.76 | 21.06 |
| | | SD | 4.052 | 2.494 | 14.446 |
| | | Median | 10.4 | 17.3 | 15 |
| | | Min, max | 8.1, 18.9 | 11.6, 18.8 | 11.3, 49.6 |

CV = coefficient of variation; ld = loading dose; MAD = multiple ascending dose; max = maximum; md = maintenance dose; min = minimum; N = total number of patients; n = number of patients with evaluable data; PK = pharmacokinetic; SD = standard deviation.
Notes:
AUC_{τ} represents AUC over the dosing interval of 14 days. Concentration values for three PK sample time points were considered physiologically implausible and were removed from the non-compartmental analysis; Patient 14072011 (Cohort 2, day 29, 120 hour), Patient 14071008 (Cohort 2, day 29, 24 hour), and Patient 14074014 (Cohort 3, day 29, 120 hour).

The elimination phase of TEV-48574 was not well defined after administration of TEV-48574 on days 1 and 15 since the 14-day sample collection periods after the first and second dose was not long enough to reliably calculate the elimination half-life. Therefore, AUC_{0-∞}, t_{1/2}, CL/F and V_{z}/F are only discussed after the last dose administered on day 29. After dosing on day 29, the TEV-48574 mean t_{1/2} values ranged from 8.3 to 8.7 days across the dose range studied. Dose level did not appear to impact the TEV-48574 elimination rate. Additionally, on day 29, mean CL/F and V_{z}/F were similar between dose levels ranging from 0.043 to 0.072 L/h and 11.79 to 21.06 L, respectively.

The accumulation in TEV-48574 serum exposure after multiple SC dosing was evaluated for each dosing interval. For cohort 1, mean observed accumulation ratio (Robs) from first to second dose (AUC_{τ}, day 15/AUC_{τ}, day 1), first to third dose (AUC_{τ}, day 29/AUC_{τ}, day 1), and second to third dose (AUC_{τ}, day 29/AUC_{τ}, day 15) was 1.472, 1.64, and 1.057, respectively. There was some accumulation from the first dose to the second and third dose but it appeared steady state was reached as there was no accumulation from the second to the third dose. For cohort 2, mean observed accumulation (Robs) from the first to the second dose, the first dose to the third dose, and second to third dose was 1.319, 1.374, and 1.107, respectively. Even with a loading dose, cohort 2 exposure appeared to slightly accumulate following this second dose. After the second dose, exposure appeared to reach steady state. For cohort 3, mean observed accumulation (Robs) from the first to second dose, first to third dose, and second to third dose, was 1.039, 1.07, and 1.054, respectively. After the loading dose, cohort 3 exposure did not appear to accumulate with subsequent dosing, suggesting steady state was reached after the loading dose administration.

The pharmacokinetics of TEV-48574 were characterized in patients with mild allergic asthma in the MAD portion of this study. Consistent with the results of the SAD portion in healthy volunteers, the pharmacokinetics of TEV-48574 in patients were characterized by slow absorption of TEV-48574 into the systemic circulation. The t_{1/2} of TEV-48574 at steady state (MAD portion) ranged from 8.3 to 8.7 days and was not impacted by dose level. Half-life estimates appear similar to those for the SAD portion of the study where TEV-48574 half-life generally exceeded 6.5 days in all cohorts. For CL/F, mean estimates were similar across the dose range tested in the MAD portion and were similar to the CL/F observed in the SAD portion. The V_{z}/F was similar across the studied dose range in the MAD portion (11.79 to 21.06 L), indicating that TEV-48574 was mostly confined to the circulatory system, consistent with what was observed in SAD portion of the study.

Across the dose range tested in the MAD portion for single and multiple-dose administration, TEV-48574 peak (Cₘₐₓ) and total (AUC_{τ}) systemic exposure increased with increasing dose. From 200 mg to 2300 mg on day 1 and from 200 mg to 1600 mg at steady state, the increase in TEV-48574 exposure was dose proportional, indicated by the 90% CI for the estimated slope for Cₘₐₓ and AUC, including unity (1.0). The MAD dose proportionality result did not match the SAD portion of the study, which suggested slight supra-proportionality when increasing dose from 36 mg to 1000 mg; however, over the 200 mg to 1000 mg dose range in the SAD portion, the increase in exposure was dose proportional.

Factors such as physiological factors, a low number of subjects per cohort, parallel design, dose volume and number of injection sites may have contributed to the larger than dose proportional increase in exposure observed when the 36 mg and 90 mg doses were included for the SAD portion of the study, but over the 200 mg to 1000 mg dose range in the SAD portion, as well across the dose range in the MAD portion, the increase in exposure was dose proportional. Furthermore, TEV-48574 half-life, CL/F, and V_{z}/F appeared similar across all doses in both the SAD and MAD portions of the study, which is suggestive of linear pharmacokinetics. Without a loading dose there was weak accumulation following the first dose in cohort 1 in the MAD portion of the study (approximately 47%). After the loading dose administered to cohort 2, there was also weak accumulation (approximately 32%), suggesting that the loading dose may have been too low to achieve steady state as rapidly as intended, whereas the loading dose used for cohort 3 achieved steady state conditions after the loading dose administration. Steady state was reached after the second dose for all dose regimens.

Single Dose Data from Additional Phase 1 Study in Healthy Participants (Japanese and Caucasian)

This study was an open-label, randomized, Phase 1 SC single-dose study to assess the PK, safety, and tolerability of TEV-48574 in healthy Japanese and Caucasian participants. On day 1, Japanese participants received 400 mg, 1000 mg or 2300 mg SC injection (8 participants per each dose level), and Caucasian participants received 2300 mg (N=8).

The first cohort included 16 Japanese subjects randomly assigned 1:1 to receive 400 or 1000 mg of TEV-48574. After a period of observation for a minimum of 3 days and up to 14 days, and provided there were no safety and tolerability concerns, the study drug administration was to continue to cohort 2, and 8 Japanese subjects received 2300 mg of TEV-48574. The study drug administration continued to cohort 3, and 8 Caucasian subjects received 2300 mg of TEV-48574. A lag time of up to 14 days from dosing cohort 2 to dosing cohort 3 was allowed to enable the investigational center time for recruitment of Japanese-matched Caucasian subjects to cohort 3.

Blood samples for TEV-48574 serum concentration measurements for pharmacokinetic assessment were collected before TEV-48574 administration and at 1, 6, and 12 hours after TEV-48574 administration on day 1 and on days 2 (24 hours), 3 (48 hours), 4 (72 hours), 5 (96 hours), 6 (120 hours), 8, 10, 15, 22, 29, 43, and 50. Additionally, a sample for TEV-48574 serum concentration assessment was attempted to be taken at time points when any severe hypersensitivity reaction (eg, anaphylaxis) was observed or in the case of a serious adverse event, where appropriate, in consultation with the medical monitor.

A total of 55 healthy subjects were screened for enrollment into this study. Of the 55 subjects screened, 32 subjects met the entry criteria and were randomized into the study: 24 Japanese and 8 Caucasian subjects. Sixteen Japanese subjects were randomized to cohort 1 and received 400 or 1000 mg of TEV-48574, 8 Japanese subjects were assigned to cohort 2 and received 2300 mg TEV-48574, and 8 Caucasian subjects were assigned to cohort 3 and received 2300 mg TEV-48574.

All 32 subjects randomized received 1 dose of TEV-48574; were evaluated for safety in the study; and qualified for the pharmacokinetic analysis set. All 32 subjects completed the study.

The majority of subjects were Asian (24 subjects [75%]) due to the design of the study. Generally, Japanese subjects weighed less than Caucasian subjects. Average weight was lower in both cohort 1 treatment groups compared to cohorts 2 and 3. Furthermore, mean weight in cohort 2 was less than cohort 3 (60.84 kg versus 62.13 kg). Body weight is a covariate for study drug exposure. Body mass index was comparable across treatment groups.

The selection of doses was supported by the TV48574-SAD-10126 safety, tolerability, pharmacokinetics, and immunogenicity study, in which single doses of 1 mg to 1000 mg and multiple doses of 200 mg to 2300 mg (loading dose) and 1600 mg (maintenance dose) were shown to have an acceptable safety profile. No deaths, serious adverse events, or PDAESI (severe hypersensitivity reactions or anaphylaxis) were reported during the single ascending dose (SAD) or multiple ascending dose (MAD) portions of the TV48574-SAD-10126 study. The pharmacokinetics of TEV-48574 in this dose range was expected to be linear, as it was in TV48574-SAD-10126, where increase in exposure was dose proportional.

As both 400 mg and 1000 mg single-dose were assessed in healthy volunteers in Study TV48574 SAD-10126, the use of similar doses administered as a single SC dose to healthy Japanese subjects was anticipated to allow a dose-based, cross-study comparison of the pharmacokinetics, safety, tolerability, and immunogenicity of TEV-48574 in these populations.

As the top dose (2300 mg) was administered to asthma patients in the MAD portion of the TV48574-SAD-10126 study, such cross-study comparison to healthy subjects was not feasible. Therefore, in this study, healthy Caucasian and Japanese subjects were administered 2300 mg for a direct comparison of the pharmacokinetics, safety, tolerability, and immunogenicity between the 2 populations.

Following single-dose SC administrations in Japanese subjects, TEV-48574 concentrations peaked between days 3 and 4 after administration in all dose levels and showed similarity in the elimination profile. Elimination half-life ranged between 7 and 9 days.

Geometric mean peak serum concentrations (Cₘₐₓ) and total systemic exposures (AUC₀₋ₜ and AUC_{0-∞}) increased with increasing dose in the dose range of 400 to 2300 mg (see Table 55).

TABLE 55
Summary of Pharmacokinetic Parameters by Cohort, Ethnic Group, and Dose Level

| | | Cohort 1 | Cohort 1 | Cohort 2 | Cohort 3 |
|---|---|---|---|---|---|
| | | Japanese | Japanese | Japanese | Caucasian |
| Pharmacokinetic | | 400 mg | 1000 mg | 2300 mg | 2300 mg |
| parameter | Statistic | (N = 8) | (N = 8) | (N = 8) | (N = 8) |
| Cₘₐₓ (μg/mL) | n | 8 | 8 | 8 | 8 |
| | Geometric | 26.69 | 50.61 | 146.36 | 144.56 |
| | Mean | | | | |
| | % CV | 30.09 | 51.97 | 34.33 | 24.75 |
| | Min, max | 14.8, 37.6 | 25.0, 103.8 | 80.6, 262.1 | 93.7, 193.5 |
| Tₘₐₓ (day) | n | 8 | 8 | 8 | 8 |
| | Median | 3.0 | 3.5 | 3.0 | 4.0 |
| | Min, max | 2, 4 | 2, 5 | 3, 5 | 3, 5 |
| AUC₀₋ₜ | n | 8 | 8 | 8 | 8 |
| (day*μg/mL) | Geometric | 329.14 | 604.04 | 1958.76 | 1904.76 |
| | Mean | | | | |
| | % CV | 30.60 | 61.45 | 20.68 | 26.61 |
| | Min, max | 212.7, 484.9 | 284.4, 1491.8 | 1419.0, 2549.2 | 1300.6, 2518.9 |
| AUC_{0-∞} | n | 8 | 8 | 8 | 8 |
| (day*μg/mL) | Geometric | 339.14 | 614.58 | 2026.77 | 1948.51 |
| | Mean | | | | |
| | % CV | 30.89 | 61.81 | 20.30 | 26.97 |
| | Min, max | 216.3, 502.6 | 287.0, 1513.1 | 1515.7, 2584.9 | 1322.8, 2602.5 |
| % AUCₑₓₜ (%) | n | 8 | 8 | 8 | 8 |
| | Mean | 2.93 | 1.70 | 3.34 | 2.24 |
| | Geometric | 2.30 | 1.14 | 2.81 | 1.95 |
| | Mean | | | | |
| | % CV | 71.94 | 90.47 | 62.51 | 46.22 |
| | Min, max | 0.7, 6.4 | 0.3, 4.3 | 1.3, 6.4 | 0.6, 3.2 |
| t½ (day) | n | 8 | 8 | 8 | 8 |
| | Mean | 8.50 | 7.19 | 9.62 | 8.95 |
| | SD | 1.982 | 1.983 | 1.552 | 1.238 |
| | Min, max | 5.0, 11.1 | 4.6, 9.6 | 7.8, 11.8 | 6.9, 10.5 |
| CL/F (mL/day) | n | 8 | 8 | 8 | 8 |
| | Mean | 1233.50 | 1849.86 | 1157.01 | 1223.07 |
| | SD | 394.259 | 930.737 | 246.112 | 351.782 |
| | Median | 1196.40 | 1834.23 | 1109.93 | 1113.88 |
| | Min, max | 795.9, 1849.5 | 660.9, 3484.3 | 889.8, 1517.5 | 883.8, 1738.7 |
| Vz/F (mL) | n | 8 | 8 | 8 | 8 |
| | Mean | 14929.96 | 18137.27 | 16247.51 | 15617.90 |
| | SD | 5079.401 | 8631.299 | 5233.128 | 4439.645 |
| | Median | 15265.09 | 16135.04 | 14673.49 | 14391.61 |
| | Min, max | 7149.1, 21180.4 | 7267.5, 30595.3 | 10734.3, 25911.9 | 11160.3, 23246.5 |

% AUCₑₓₜ = percentage of the extrapolated area to infinity in relation to the total area under the curve; AUC₀₋ₜ = area under the serum concentration-time curve from time 0 to the time of the last measurable TEV-48574 concentration; AUC_{0-∞} = area under the serum concentration-time curve extrapolated to infinity; CL/F = apparent total body clearance; Cₘₐₓ = maximum observed serum drug concentration; CV = coefficient of variation; Max = maximum; Min = minimum; N = total number of subjects; n = number of subjects with evaluable data; SD = standard deviation; SE = standard error; t_{1/2} = terminal phase (apparent elimination) half-life; Tₘₐₓ = time to maximum observed serum drug concentration; V_{z}/F = apparent volume of distribution during terminal phase.

Dose proportionality analysis over the dose range of 400 to 2300 mg indicates linear exposure with increasing dose in Japanese subjects.

Pharmacokinetic profile of TEV-48574 in Japanese subjects was similar to pharmacokinetic profile in Caucasian subjects across the absorption, distribution, and elimination phases; pharmacokinetic parameters in Japanese subjects were comparable to Caucasian subjects in the 2300 mg dose cohorts and consistent with pharmacokinetic parameters in Caucasian subjects obtained from previous first-in-human (FIH) TV48574-SAD-10126 study.

A comparison of pooled dose-normalized pharmacokinetic parameters in Japanese subjects from all 3 cohorts to pharmacokinetic parameters from 2300 mg dose cohort in Caucasian subjects was performed. Cₘₐₓ and AUC_{0-∞} were similar between the compared groups.

Phase 1 Study in healthy subjects (Japanese and Caucasian) demonstrated comparability in the pharmacokinetic profile for TEV-48574 between Japanese and Caucasian subjects. The plots for mean serum concentration and individual subject serum concentration profiles fully overlap between the 2 ethnic groups at the 2300 mg dose.

Exposure parameters (Cₘₐₓ, AUC₀₋ₜ, and AUC_{0-∞}) in Japanese subjects were comparable to Caucasian subjects in the 2300 mg dose cohorts and consistent with pharmacokinetic parameters in Caucasian subjects obtained from previous FIH TV48574-SAD-10126 study. Mean half-life was similar between the 2 ethnic groups and consistent with half-life reported in TV48574-SAD-10126, ranging between 6 to 9 days across studies. CL/F and V_{z}/F are consistent with values reported in TV48574-SAD-10126.

Exploratory assessment for dose proportionality suggests linear exposure with an increasing dose in Japanese subjects for the dose range of 400 to 2300 mg. This is consistent with data reported in the TV48574-SAD-10126 study, where exposure was dose proportional for the dose range of 200 to 2300 mg.

Based on these results, the pharmacokinetics of TEV-48574 is not expected to be significantly affected by ethnic factors.

### Methods

Data preparation has been performed using SAS Version 9.4. Exploratory data analysis and presentation of data has been performed by R version 3.5.2 and various R packages. Population modeling was performed using nonlinear mixed-effect platform NONMEM version 7.3 and Monolix version 2018R. All the analyses were performed in a validated computerized system Advanced Computational Environment (ACE) and all the analysis files were stored in a validated storage system Bitbucket.

A previously developed population PK model consisting of 1-compartment with linear elimination, lag time and body weight at baseline as a covariate on clearance and volume of distribution was used as a starting point for models described herein.

### Population PK Modeling and Simulations of TEV-48574 and RVT-3101

During structural PK model development, several variations of the one-compartment model were evaluated. A one-compartment model with first-order absorption, linear elimination, body weight at baseline as a covariate on clearance and volume of distribution, best described the serum TEV-48574 concentration-time data (see, Table 56).

TABLE 56
Population PK model parameters

| | | | | |
|---|---|---|---|---|
| PARAMETER | VALUE | RSE | SHRINKAGE | COMMENT |

Typical parameters

| | | | | |
|---|---|---|---|---|
| CL | 1.24 | 5.3% | — | (L/day) Apparent clearance |
| Vc | 13 | 4.49% | — | (L) Apparent volume of distribution |
| ka | 0.597 | 5.44% | | (1/day) Absorption rate parameter |
| Fabs1 | 1 (FIX) | — | — | (.) Bioavailability |

Inter-individual variability

| | | | | |
|---|---|---|---|---|
| omega(CL) | 0.408 | 6.95% | 7.6% | LogNormal |
| omega(Vc) | 0.327 | 8.73% | 14.4% | LogNormal |
| omega(ka) | 0.353 | 12.1% | 31.8% | LogNormal |
| omega(Fabs1) | 0 (FIX) | — | — | LogNormal |

Parameter-Covariate relationships

| | | | | |
|---|---|---|---|---|
| beta_CL(WEIGHTBL) | 0.712 | 34.2% | — | |
| beta_Vc(WEIGHTBL) | 0.788 | 22.1% | — | |
| Residual Variability | | | | |
| error_ADD1 | 1.18 | 0.523% | — | Additive Error (ug/mL) - Plasma |
| | | | | concentration |
| error_PROP1 | 0.184 | 1.26% | — | Proportional Error (fraction) - |
| | | | | Plasma concentration |
| Objective function | 6217 | — | — | |
| AIC | 6237 | — | — | |
| BIC | 6292 | — | — | |

The population PK model of RVT-3101 was constructed based on the model described in Danese S et al. Clin. Gastroenterol. Hepatol. 2021; 19 (11): 2324-2332.e6, which is incorporated herein by reference in its entirety. RVT-3101 (also known as PF-06480605) is disclosed as antibody 1D1 1.31 and further described in PCT Patent Publications WO 2015/073580 and WO 2021/260577). The Danese et al. model structure included 2-compartments with a clearance of 0.00575 L/hr (8.26% relative standard error (RSE)), a central volume of distribution of 3.02 L (4.74% RSE), an inter-compartamental clearance of 0.0141 L/hr (4.53% RSE), a peripheral volume of distribution of 5.78 L (1.94% RSE), a SC bioavailability of 67.5%, an absorption rate of 0.00671 hr-1, and bodyweight as a significant covariate on clearance and central volume with power exponents of 0.746 (49.1% RSE) and 0.578 (44.8% RSE), respectively.

Both of the TEV-48574 and RVT-3101 population PK models were used in simulation mode to anticipate various doses and dosing regimens and compare exposures between the two compounds.

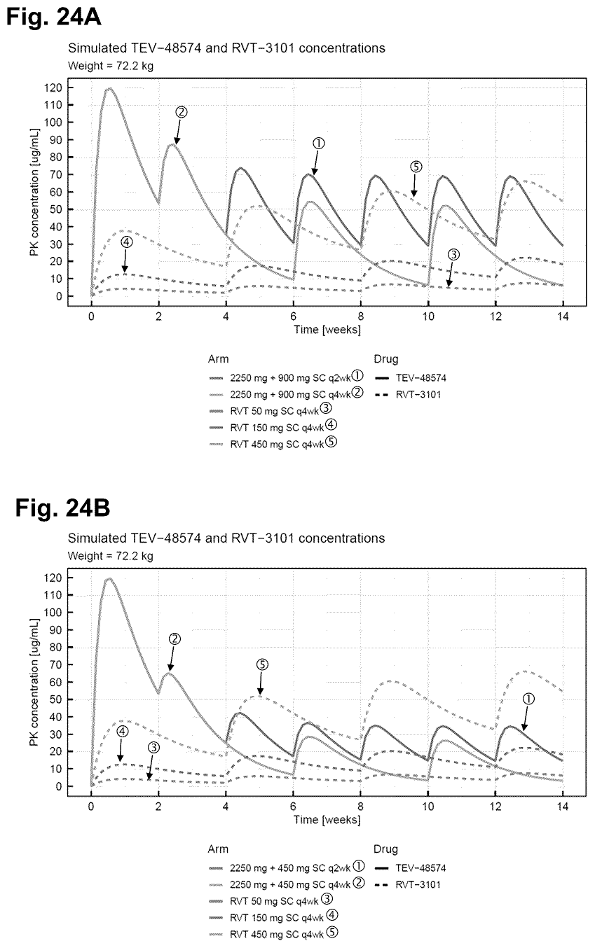

As shown in FIG. 24A, simulated TEV-48574 concentrations for a 2250 mg loading dose and either a Q2W or Q4W maintenance regimen of 900 mg were compared to RVT-3101 anticipated exposures following administration of 50 mg, 150 mg, or 450 mg of RVT-3101 on a Q4W regimen.

As shown in FIG. 24B, simulated TEV-48574 concentrations for a 2250 mg loading dose and either a Q2W or Q4W maintenance regimen of 450 mg were compared to RVT-3101 anticipated exposures following administration of 50 mg, 150 mg, or 450 mg of RVT-3101 on a Q4W regimen.

The results from the two PK simulations support the TEV-48574 regimen described in the clinical studies of Examples 1, 4 and 9. The results indicate that following a 2250 mg loading dose, induction and/or maintenance doses of TEV-48574 at both 450 mg and 900 mg administered every 4 weeks will reach exposure levels anticipated to demonstrate clinical effects.

### Example 9: Alternate Clinical Evaluation of TEV-48574 in Moderate to Severe Ulcerative Colitis or Moderate to Severe Crohn's Disease

### Initial Treatment Period

TEV-48574 is assessed in a Phase 2b, randomized, double-blind, dose-ranging study to determine the pharmacokinetics, efficacy, safety, and tolerability of TEV-48574 in adult patients with moderate to severe ulcerative colitis (UC) or Crohn's disease (CD) per the description in Example 1 with the following changes.

The study demonstrates the efficacy and dose response of 2 different dose regimens of TEV-48574 subcutaneously (sc) administered every 2 weeks (Q2W) in adult patients with inflammatory bowel disease (IBD) (moderate to severe UC or CD) as assessed by induction of clinical remission (UC) and endoscopic response (CD) at week 14.

Approximately 570 patients are screened to achieve approximately 240 randomized patients (approximately 120 patients with UC and 120 patients with CD). Patients who meet all the inclusion criteria are randomly assigned to receive TEV-48574 (single loading dose/6 induction doses): 2250/900 mg, 2250/450 mg, or placebo to match TEV-48574, in a 1:1:1 ratio, stratified by diagnosis (UC or CD) and previous exposure, failure of biologics and small molecules (including JAK inhibitors and SIP receptor modulators for UC) or naive to biologics and small molecule therapy. Thus, patients receive the following regimens as a SC administration using a commercial syringe-based SC infusion system:
TEV-48574 2250 mg (single loading dose)/900 mg (6 induction doses)
TEV-48574 2250 mg (single loading dose)/450 mg (6 induction doses)
Placebo to match TEV-48574

The long-term efficacy and safety of TEV-48574 is then demonstrated in patients who had moderately to severely active UC or CD at the beginning of the study and who also showed a clinical response and/or clinical remission (based on complete Mayo score for the UC and CDAI for the CD subjects) to initial treatment with TEV-48574 in the study. These patients will receive either 450 mg Q4W or 900 mg Q4W for the duration of the administration (described below in the Long-Term Extension Study section).

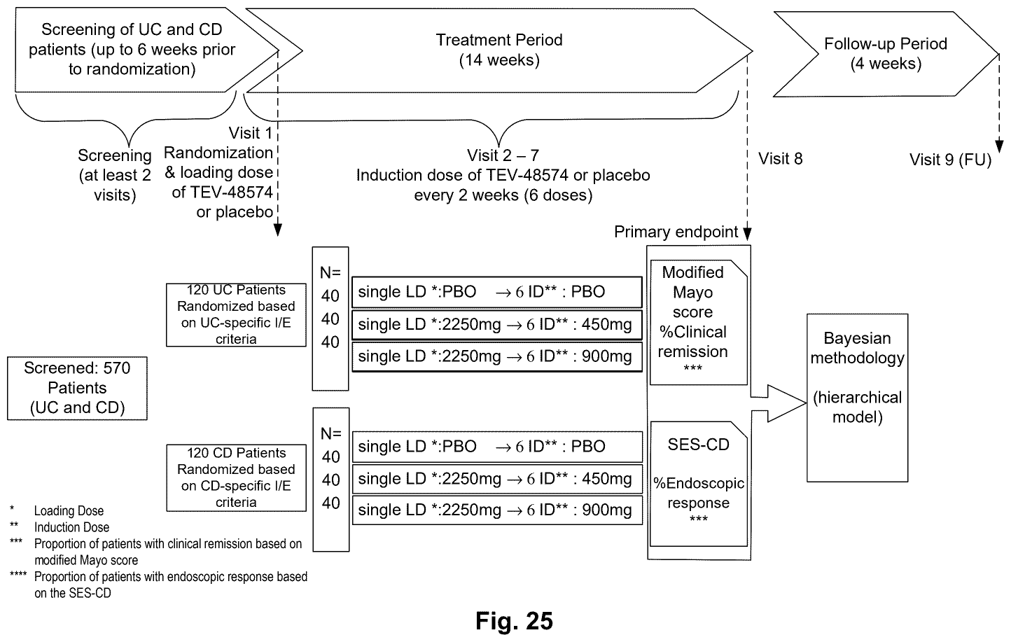

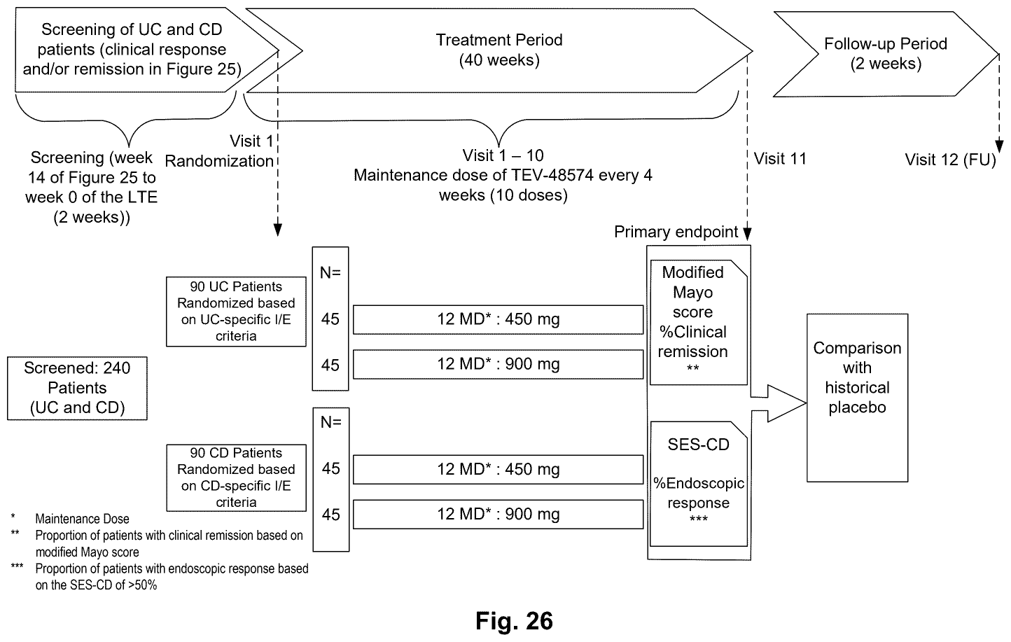

The study schematic diagram is presented in FIG. 25.

Randomization: Patients satisfying the eligibility criteria at the end of the screening period are randomized in a 1:1:1 ratio (stratified by diagnosis [UC or CD] and previous exposure, failure of biologics and small molecules [including JAK inhibitors and S1P receptor modulators for UC] or naive to biologics and small molecule therapy) to 1 of 3 treatment groups for the double-blind treatment period (Table 57).

All treatments are administered in a double-blind fashion as 1 SC infusion Q2W.

TABLE 57
Randomization Scheme

| Randomization | Planned approximate | |
|---|---|---|
| ratio | number randomized | Treatment (Q2W) |
| 1 | 40 UC + 40 CD | TEV-48574 2250 mg (single loading |
| | | dose)/900 mg (6 induction doses) |
| 1 | 40 UC + 40 CD | TEV-48574 2250 mg (single loading |
| | | dose)/450 mg (6 induction doses) |
| 1 | 40 UC + 40 CD | Placebo |

CD = Crohn's disease; Q2W = every 2 weeks; UC = ulcerative colitis.

The results are analyzed as discussed in Example 1 to demonstrate the efficacy and dose response of 2 different dose regimens of TEV-48574 subcutaneous (SC) administered every 2 weeks (Q2W) in adult patients with IBD (moderate to severe UC or CD) as assessed by induction of clinical remission (UC) and endoscopic response (CD) at week 14.

### Long-Term Extension Study

TEV-48574 is assessed in a Phase 2b, randomized, double-blind long-term extension (LTE) study to evaluate the pharmacokinetics, efficacy, safety, and tolerability of TEV-48574 in adult patients with moderate to severe ulcerative colitis (UC) or Crohn's disease (CD) who achieved clinical response and/or clinical remission during the treatment phase of the 14-week Initial Treatment Period (ITP) described above in this Example (ITP). The study schematic diagram is presented in FIG. 26. This LTE study is performed according per the description in Example 4 with the following changes. The study is a 40-week study.

Eligible patients meet all of the following inclusion criteria:
Adults of male and female sex (without restrictions on gender) who at the time of informed consent achieved clinical response and/or clinical remission in the 14-week ITP.
   Clinical response assessed by decrease from baseline in modified (9-point rectal bleeding, stool frequency, and endoscopy) Mayo score by >2 points and at least 30%, with a decrease in rectal bleeding subscore of ≥1 point or an absolute subscore of 0 or 1 at week 14 of the ITP in patients with moderate to severe UC.
   Clinical remission at week 14 of the ITP in patients with moderate to severe UC. Clinical remission is a modified (9-point rectal bleeding, stool frequency, and endoscopy) Mayo score of ≤2 points, which is defined by:
      stool frequency subscore of 0 or 1,
      rectal bleeding subscore of 0, and
      endoscopic subscore of 0 or 1, where a score of 1 does not include “friability.”
   Clinical response assessed by ≥100-point decrease in Crohn's Disease Activity Index (CDAI) score from baseline week 14 of the ITP in patients with moderate to severe CD.
   Clinical remission defined as a CDAI score <150 at week 14 of the ITP in patients with moderate to severe CD.

Patients who meet all the inclusion criteria are randomly assigned to receive TEV-48574 (10 maintenance doses): 900 mg or 450 mg in a 1:1 ratio, stratified by diagnosis (UC or CD). The data provided herein, e.g., in Examples 7 and 8, indicates that a 2250 mg loading dose followed by maintenance doses of TEV-48574 at 450 mg or 900 mg every 4 weeks will reach exposure levels anticipated to demonstrate clinical effects.

### General Design

The study includes a 40-week treatment period and a 2-week follow-up period. Randomization: Patients satisfying the eligibility criteria at the end of treatment visit (EOT) in the 14-week study described in the ITP above are randomized in a 1:1 ratio (stratified by diagnosis [UC or CD], to 1 of 2 treatment groups for the double-blind treatment period (See Table 58 below). Approximately 240 patients will be screened (after week 14 of ITP). The total number of patients in the LTE will be approximately 90 patients per indication (approximately 45 patients in each treatment group).

All study treatments are administered in a double-blind fashion as 1 SC infusion Q4W.

TABLE 58
Randomization Scheme

| Randomization | Planned approximate | |
|---|---|---|
| ratio | number randomized | Treatment (Q4W) |
| 1 | 45 UC + 45 CD | TEV-48574 450 mg |
| | | (10 maintenance doses) |
| 1 | 45 UC + 45 CD | TEV-48574 900 mg |
| | | (10 maintenance doses) |

CD = Crohn's disease; Q4W = every 4 weeks; UC = ulcerative colitis.

During the 40-week treatment period, patients visit the site Q4W on days 1, 29, 57, 85, 113, 141, 169, 197, 225, and 253 (±3 days) for TEV-48574 administration (10 visits) and on day 281 (±3 days) for endpoints assessment. After the end of the 40-week treatment period, patients return to the site for a follow-up visit (day 309 [±3 days]). Patients who complete the follow-up visit are considered to have completed the study.

Patients are allowed to use the following medications as detailed below:
Concomitant use of oral 5-ASA or sulfasalazine. Dose must be stable through the transition period and throughout the study (week 40).
A stable dose of oral corticosteroids (prednisone equivalent of up to 20 mg/day; budesonide of up to 9 mg/day) through the transition period and throughout the study (week 40).
A stable dose of immunosuppressant drugs (methotrexate≤25 mg intramuscular or SC once weekly or ≤15 mg per os once weekly; 6-MP≤1.5 mg/kg/day; or AZA ≤2.5 mg/kg/day).

The following endpoints are analyzed to demonstrate a lack of immunogenicity:
Treatment-emergent anti-drug antibody (ADA) results and responses: change from baseline (ITP) and throughout the study
Neutralizing ADA in ADA positive patients throughout the study
Impact of the presence of ADAs on pharmacokinetics and clinical safety will be assessed if applicable.

The following efficacy endpoints are analyzed to demonstrate efficacy of TEV-48574:
Clinical remission based on modified (9-point rectal bleeding, stool frequency, and endoscopy) Mayo score of ≤2 points defined by a stool frequency subscore of 0 or 1, rectal bleeding subscore of 0, and an endoscopic subscore of 0 or 1, where a score of 1 does not include “friability” at week 40 in patients with UC
Endoscopic response, defined as a decrease in Simple Endoscopic Score for Crohn's Disease (SES-CD) of at least 50% from the ITP baseline at week 40 in patients with CD.

The following endpoints are also analyzed to demonstrate efficacy of TEV-48574:
Clinical response, based on modified (9-point rectal bleeding, stool frequency, and endoscopy) Mayo score of at least 2 points AND at least a 30% reduction from the ITP with either a decrease in rectal bleeding subscore of at least 1 or an absolute rectal bleeding subscore of less than or equal to 1 at week 40 in patients with UC
Endoscopic improvement from the ITP baseline based on Mayo endoscopic subscore of 0 or 1 at week 40 in patients with UC
Endoscopic remission based on Mayo endoscopic subscore of 0 at week 40 in patients with UC
Clinical response defined as decrease from the ITP baseline of at least 50% in 2-item patient-reported outcome (PRO2; rectal bleeding and stool frequency) at weeks 0, 4, 8, 12, 16, 20, 24, 28, 32, 36 and 40 in patients with UC
Clinical remission based on PRO2 (rectal bleeding-0 and stool frequency=0) at weeks 0, 4, 8, 12, 16, 20, 24, 28, 32, 36 and 40 in patients with UC
Clinical response based on Crohn's Disease Activity Index (CDAI): ≥100-point decrease in CDAI score from the ITP baseline at week 24 in patients with CD at weeks 0, 4, 8, 12, 16, 20, 24, 28, 32, 36, and 40.
Clinical remission based on CDAI score <150 at week 0, 4, 8, 12, 16, 20, 24, 28, 32, 36, and 40 in patients with CD.
Clinical response defined as decrease in PRO2 from the ITP baseline of at least 50% in PRO2 at weeks 0, 4, 8, 12, 16, 20, 24, 28, 32, 36 and 40 in patients with CD
Clinical remission based on PRO2 (abdominal pain≤1 and stool frequency≤3) at weeks 0, 4, 8, 12, 16, 20, 24, 28, 32, 36, and 40 in patients with CD.
Histologic response (>50% decrease) in Global Histologic Activity Score from the ITP baseline at week 40 in patients with CD.

It is to be appreciated that the Detailed Description section, and not the Summary and Abstract sections, is intended to be used to interpret the claims. The Summary and Abstract sections can set forth one or more but not all exemplary aspects of the present invention as contemplated by the inventor(s), and thus, are not intended to limit the present invention and the appended claims in any way.

The foregoing description of the specific aspects will so fully reveal the general nature of the invention that others can, by applying knowledge within the skill of the art, readily modify and/or adapt for various applications such specific aspects, without undue experimentation, without departing from the general concept of the present invention. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary aspects, but should be defined only in accordance with the following claims and their equivalents.

Various publications, including patents, published applications, accession numbers, technical articles and scholarly articles are cited throughout the specification. Each of these cited publications is incorporated by reference, in its entirety and for all purposes, in this document.

Sequence Table:

| SEQ ID NO: | Description | Sequence |
|---|---|---|
| 1 | 320-587 Heavy | GYTFTSYDIN |
| | Chain CDR1 | |
| 2 | 320-587 Heavy | WLNPNSGYTG |
| | Chain CDR2 | |
| 3 | 320-587 Heavy | EVPETAAFEY |
| | Chain CDR3 | |
| 4 | 320-587 Light Chain | TSSSSDIGAGLGVH |
| | CDR1 | |
| 5 | 320-587 Light Chain | GYYNRPS |
| | CDR2 | |
| 6 | 320-587 Light Chain | QSWDGTLSAL |
| | CDR3 | |
| 7 | 320-587 Heavy | QVQLVQSGAEVKKPGASVKVSCKASGYTFTSY |
| | Chain Variable | DINWVRQAPGQGLEWMGWLNPNSGYTGYAQK |
| | Region | FQGRVTMTADRSTSTAYMELSSLRSEDTAVYY |
| | | CAREVPETAAFEYWGQGTLVTVSS |
| 8 | 320-587 Light Chain | QSVLTQPPSVSGAPGQRVTISCTSSSSDIGAGLG |
| | Variable Region | VHWYQQLPGTAPKLLIEGYYNRPSGVPDRFSGS |
| | | KSGTSASLTITGLLPEDEGDYYCQSWDGTLSAL |
| | | FGGGTKLTVLG |
| 9 | 320-587 Heavy | QVQLVQSGAEVKKPGASVKVSCKASGYTFTSY |
| | Chain | DINWVRQAPGQGLEWMGWLNPNSGYTGYAQK |
| | | FQGRVTMTADRSTSTAYMELSSLRSEDTAVYY |
| | | CAREVPETAAFEYWGQGTLVTVSSASTKGPSVF |
| | | PLAPSSKSTSGGTAALGCLVKDYFPEPVTVSWN |
| | | SGALTSGVHTFPAVLQSSGLYSLSSVVTVPSSSL |
| | | GTQTYICNVNHKPSNTKVDKKVEPKSCDKTHT |
| | | CPPCPAPELLGGPSVFLFPPKPKDTLMISRTPEVT |
| | | CVVVDVSHEDPEVKFNWYVDGVEVHNAKTKP |
| | | REEQYNSTYRVVSVLTVLHQDWLNGKEYKCK |
| | | VSNKALPAPIEKTISKAKGQPREPQVYTLPPSRD |
| | | ELTKNQVSLTCLVKGFYPSDIAVEWESNGQPEN |
| | | NYKTTPPVLDSDGSFFLYSKLTVDKSRWQQGN |
| | | VFSCSVMHEALHNHYTQKSLSLSPG |
| 10 | 320-587 Light Chain | QSVLTQPPSVSGAPGQRVTISCTSSSSDIGAGLG |
| | | VHWYQQLPGTAPKLLIEGYYNRPSGVPDRFSGS |
| | | KSGTSASLTITGLLPEDEGDYYCQSWDGTLSAL |
| | | FGGGTKLTVLGQPKAAPSVTLFPPSSEELQANK |
| | | ATLVCLISDFYPGAVTVAWKADSSPVKAGVETT |
| | | TPSKQSNNKYAASSYLSLTPEQWKSHRSYSCQV |
| | | THEGSTVEKTVAPTECS |
| 11 | 320-179 Heavy | QVQLVQSGAEVKKPGASVKVSCKASGYTFTSY |
| | Chain Variable | DINWVRQAPGQGLEWMGWLNPNSGNTGYAQK |
| | Region | |
| | | FQGRVTMTADRSTSTAYMELSSLRSEDTAVYY |
| | | CAREVPETAAFEYWGQGTLVTVSS |
| 12 | 320-179 Light Chain | QSVLTQPPSVSGAPGQRVTISCTSSSSDIGAGLG |
| | Variable Region | VHWYQQLPGTAPKLLIEGYYNRPSGVPDRFSGS |
| | | KSGTSASLTITGLLPEDEGDYYCQSYDGTLSALF |
| | | GGGTKLTVLG |
| 13 | IgG1 HC constant | ASTKGPSVFPLAPSSKSTSGGTAALGCLVKDYFP |
| | region | EPVTVSWNSGALTSGVHTFPAVLQSSGLYSLSS |
| | | VVTVPSSSLGTQTYICNVNHKPSNTKVDKKVEP |
| | | KSCDKTHTCPPCPAPELLGGPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| | | K |
| 14 | IgG1 dK HC | ASTKGPSVFPLAPSSKSTSGGTAALGCLVKDYFP |
| | constant region | EPVTVSWNSGALTSGVHTFPAVLQSSGLYSLSS |
| | | VVTVPSSSLGTQTYICNVNHKPSNTKVDKKVEP |
| | | KSCDKTHTCPPCPAPELLGGPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| 15 | IgG1 | ASTKGPSVFPLAPSSKSTSGGTAALGCLVKDYFP |
| | 252Y/254T/256E | EPVTVSWNSGALTSGVHTFPAVLQSSGLYSLSS |
| | HC constant region | VVTVPSSSLGTQTYICNVNHKPSNTKVDKKVEP |
| | | KSCDKTHTCPPCPAPELLGGPSVFLFPPKPKDTL |
| | | YITREPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| | | K |
| 16 | IgG1 dK + | ASTKGPSVFPLAPSSKSTSGGTAALGCLVKDYFP |
| | 252Y/254T/256E | EPVTVSWNSGALTSGVHTFPAVLQSSGLYSLSS |
| | HC constant region | VVTVPSSSLGTQTYICNVNHKPSNTKVDKKVEP |
| | | KSCDKTHTCPPCPAPELLGGPSVFLFPPKPKDTL |
| | | YITREPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| 17 | IgG1 L234A, | ASTKGPSVFPLAPSSKSTSGGTAALGCLVKDYFP |
| | L235A, G237A HC | EPVTVSWNSGALTSGVHTFPAVLQSSGLYSLSS |
| | constant region | VVTVPSSSLGTQTYICNVNHKPSNTKVDKKVEP |
| | | KSCDKTHTCPPCPAPEAAGAPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| | | K |
| 18 | IgG1 dK + L234A, | ASTKGPSVFPLAPSSKSTSGGTAALGCLVKDYFP |
| | L235A, G237A HC | EPVTVSWNSGALTSGVHTFPAVLQSSGLYSLSS |
| | constant region | VVTVPSSSLGTQTYICNVNHKPSNTKVDKKVEP |
| | | KSCDKTHTCPPCPAPEAAGAPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSREEMTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| 19 | IgG1 L235A/G237A | ASTKGPSVFPLAPSSKSTSGGTAALGCLVKDYFP |
| | HC constant region | EPVTVSWNSGALTSGVHTFPAVLQSSGLYSLSS |
| | | VVTVPSSSLGTQTYICNVNHKPSNTKVDKKVEP |
| | | KSCDKTHTCPPCPAPELAGAPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| | | K |
| 20 | IgG1 dK + | ASTKGPSVFPLAPSSKSTSGGTAALGCLVKDYFP |
| | L235A/G237A HC | EPVTVSWNSGALTSGVHTFPAVLQSSGLYSLSS |
| | constant region | VVTVPSSSLGTQTYICNVNHKPSNTKVDKKVEP |
| | | KSCDKTHTCPPCPAPELAGAPSVFLFPPKPKDTL |
| | | MISRTPEVTCVVVDVSHEDPEVKFNWYVDGVE |
| | | VHNAKTKPREEQYNSTYRVVSVLTVLHQDWLN |
| | | GKEYKCKVSNKALPAPIEKTISKAKGQPREPQV |
| | | YTLPPSRDELTKNQVSLTCLVKGFYPSDIAVEW |
| | | ESNGQPENNYKTTPPVLDSDGSFFLYSKLTVDK |
| | | SRWQQGNVFSCSVMHEALHNHYTQKSLSLSPG |
| 21 | IgG2 HC constant | ASTKGPSVFPLAPCSRSTSESTAALGCLVKDYFP |
| | region | EPVTVSWNSGALTSGVHTFPAVLQSSGLYSLSS |
| | | VVTVPSSNFGTQTYTCNVDHKPSNTKVDKTVE |
| | | RKCCVECPPCPAPPVAGPSVFLFPPKPKDTLMIS |
| | | RTPEVTCVVVDVSHEDPEVQFNWYVDGVEVHN |
| | | AKTKPREEQFNSTFRVVSVLTVVHQDWLNGKE |
| | | YKCKVSNKGLPAPIEKTISKTKGQPREPQVYTLP |
| | | PSREEMTKNQVSLTCLVKGFYPSDIAVEWESNG |
| | | QPENNYKTTPPMLDSDGSFFLYSKLTVDKSRWQ |
| | | QGNVFSCSVMHEALHNHYTQKSLSLSPGK |
| 22 | IgG2 dK HC | ASTKGPSVFPLAPCSRSTSESTAALGCLVKDYFP |
| | constant region | EPVTVSWNSGALTSGVHTFPAVLQSSGLYSLSS |
| | | VVTVPSSNFGTQTYTCNVDHKPSNTKVDKTVE |
| | | RKCCVECPPCPAPPVAGPSVFLFPPKPKDTLMIS |
| | | RTPEVTCVVVDVSHEDPEVQFNWYVDGVEVHN |
| | | AKTKPREEQFNSTFRVVSVLTVVHQDWLNGKE |
| | | YKCKVSNKGLPAPIEKTISKTKGQPREPQVYTLP |
| | | PSREEMTKNQVSLTCLVKGFYPSDIAVEWESNG |
| | | QPENNYKTTPPMLDSDGSFFLYSKLTVDKSRWQ |
| | | QGNVFSCSVMHEALHNHYTQKSLSLSPG |
| 23 | IgG2 A330S/P331S | ASTKGPSVFPLAPCSRSTSESTAALGCLVKDYFP |
| | HC constant region | EPVTVSWNSGALTSGVHTFPAVLQSSGLYSLSS |
| | | VVTVPSSNFGTQTYTCNVDHKPSNTKVDKTVE |
| | | RKCCVECPPCPAPPVAGPSVFLFPPKPKDTLMIS |
| | | RTPEVTCVVVDVSHEDPEVQFNWYVDGVEVHN |
| | | AKTKPREEQFNSTFRVVSVLTVVHQDWLNGKE |
| | | YKCKVSNKGLPSSIEKTISKTKGQPREPQVYTLP |
| | | PSREEMTKNQVSLTCLVKGFYPSDIAVEWESNG |
| | | QPENNYKTTPPMLDSDGSFFLYSKLTVDKSRWQ |
| | | QGNVFSCSVMHEALHNHYTQKSLSLSPGK |
| 24 | IgG2 dK + | ASTKGPSVFPLAPCSRSTSESTAALGCLVKDYFP |
| | A330S/P331S HC | EPVTVSWNSGALTSGVHTFPAVLQSSGLYSLSS |
| | constant region | VVTVPSSNFGTQTYTCNVDHKPSNTKVDKTVE |
| | | RKCCVECPPCPAPPVAGPSVFLFPPKPKDTLMIS |
| | | RTPEVTCVVVDVSHEDPEVQFNWYVDGVEVHN |
| | | AKTKPREEQFNSTFRVVSVLTVVHQDWLNGKE |
| | | YKCKVSNKGLPSSIEKTISKTKGQPREPQVYTLP |
| | | PSREEMTKNQVSLTCLVKGFYPSDIAVEWESNG |
| | | QPENNYKTTPPMLDSDGSFFLYSKLTVDKSRWQ |
| | | QGNVFSCSVMHEALHNHYTQKSLSLSPG |
| 25 | IgG4 HC constant | ASTKGPSVFPLAPCSRSTSESTAALGCLVKDYFP |
| | region | EPVTVSWNSGALTSGVHTFPAVLQSSGLYSLSS |
| | | VVTVPSSSLGTKTYTCNVDHKPSNTKVDKRVES |
| | | KYGPPCPPCPAPEFLGGPSVFLFPPKPKDTLMISR |
| | | TPEVTCVVVDVSQEDPEVQFNWYVDGVEVHN |
| | | AKTKPREEQFNSTYRVVSVLTVLHQDWLNGKE |
| | | YKCKVSNKGLPSSIEKTISKAKGQPREPQVYTLP |
| | | PSQEEMTKNQVSLTCLVKGFYPSDIAVEWESNG |
| | | QPENNYKTTPPVLDSDGSFFLYSRLTVDKSRWQ |
| | | EGNVFSCSVMHEALHNHYTQKSLSLSLGK |
| 26 | IgG4 dK + S228P | ASTKGPSVFPLAPCSRSTSESTAALGCLVKDYFP |
| | HC constant region | EPVTVSWNSGALTSGVHTFPAVLQSSGLYSLSS |
| | | VVTVPSSSLGTKTYTCNVDHKPSNTKVDKRVES |
| | | KYGPPCPPCPAPEFLGGPSVFLFPPKPKDTLMISR |
| | | TPEVTCVVVDVSQEDPEVQFNWYVDGVEVHN |
| | | AKTKPREEQFNSTYRVVSVLTVLHQDWLNGKE |
| | | YKCKVSNKGLPSSIEKTISKAKGQPREPQVYTLP |
| | | PSQEEMTKNQVSLTCLVKGFYPSDIAVEWESNG |
| | | QPENNYKTTPPVLDSDGSFFLYSRLTVDKSRWQ |
| | | EGNVFSCSVMHEALHNHYTQKSLSLSLG |
| 27 | IgG4 S228P + | ASTKGPSVFPLAPCSRSTSESTAALGCLVKDYFP |
| | 252Y/254T/256E | EPVTVSWNSGALTSGVHTFPAVLQSSGLYSLSS |
| | HC constant region | VVTVPSSSLGTKTYTCNVDHKPSNTKVDKRVES |
| | | KYGPPCPPCPAPEFLGGPSVFLFPPKPKDTLYITR |
| | | EPEVTCVVVDVSQEDPEVQFNWYVDGVEVHN |
| | | AKTKPREEQFNSTYRVVSVLTVLHQDWLNGKE |
| | | YKCKVSNKGLPSSIEKTISKAKGQPREPQVYTLP |
| | | PSQEEMTKNQVSLTCLVKGFYPSDIAVEWESNG |
| | | QPENNYKTTPPVLDSDGSFFLYSRLTVDKSRWQ |
| | | EGNVFSCSVMHEALHNHYTQKSLSLSLGK |
| 28 | IgG4 dK + S228P + | ASTKGPSVFPLAPCSRSTSESTAALGCLVKDYFP |
| | 252Y/254T/256E | EPVTVSWNSGALTSGVHTFPAVLQSSGLYSLSS |
| | HC constant region | VVTVPSSSLGTKTYTCNVDHKPSNTKVDKRVES |
| | | KYGPPCPPCPAPEFLGGPSVFLFPPKPKDTLYITR |
| | | EPEVTCVVVDVSQEDPEVQFNWYVDGVEVHN |
| | | AKTKPREEQFNSTYRVVSVLTVLHQDWLNGKE |
| | | YKCKVSNKGLPSSIEKTISKAKGQPREPQVYTLP |
| | | PSQEEMTKNQVSLTCLVKGFYPSDIAVEWESNG |
| | | QPENNYKTTPPVLDSDGSFFLYSRLTVDKSRWQ |
| | | EGNVFSCSVMHEALHNHYTQKSLSLSLG |
| 29 | Lambda LC constant | QPKAAPSVTLFPPSSEELQANKATLVCLISDFYP |
| | region | GAVTVAWKADSSPVKAGVETTTPSKQSNNKYA |
| | | ASSYLSLTPEQWKSHRSYSCQVTHEGSTVEKTV |
| | | APTECS |
| 30 | 320-587 Heavy | caggtgcagctggtgcagtccggcgccgaggtgaagaaacccggcgcct |
| | Chain Variable | ccgtgaaggtgtcctgcaaggccagcggctacaccttcacctcctacgaca |
| | Region | tcaactgggtgaggcaggcccccggccagggcctggagtggatgggctg |
| | | gctgaaccccaactccggctacaccggctacgcccagaagttccagggca |
| | | gggtgaccatgaccgccgacaggtccacctccaccgcctacatggagctg |
| | | tccagcctgaggtccgaggacaccgccgtgtactattgcgccagggaggt |
| | | gcccgagaccgctgccttcgagtactggggccagggcaccctggtgacc |
| | | gtgtccagc |
| 31 | 320-587 Heavy | caggtgcagctggtgcagtccggcgccgaggtgaagaaacccggcgcct |
| | Chain | ccgtgaaggtgtcctgcaaggccagcggctacaccttcacctcctacgaca |
| | | tcaactgggtgaggcaggcccccggccagggcctggagtggatgggctg |
| | | gctgaaccccaactccggctacaccggctacgcccagaagttccagggca |
| | | gggtgaccatgaccgccgacaggtccacctccaccgcctacatggagctg |
| | | tccagcctgaggtccgaggacaccgccgtgtactattgcgccagggaggt |
| | | gcccgagaccgctgccttcgagtactggggccagggcaccctggtgacc |
| | | gtgtccagcgcctccaccaagggccccagcgtgttccccctggcccccag |
| | | ctccaagtccaccagcggcggaaccgccgctctgggctgcctggtgaag |
| | | gactacttccccgagcccgtgaccgtgagctggaactccggcgccctgac |
| | | ctccggcgtgcacaccttccccgccgtgctgcagtccagcggcctgtactc |
| | | cctgagctccgtggtcaccgtgccctcctctagcctgggcacccagaccta |
| | | catctgcaacgtgaaccacaagccctccaacaccaaggtggacaaaaag |
| | | gtggagcccaagtcctgcgacaagactcacacctgtcctccctgccccgc |
| | | ccccgagctgctcggcggaccctccgtgttcctgttcccacccaagcccaa |
| | | ggacaccctgatgatctccaggacccccgaggtgacctgcgtggtcgtgg |
| | | acgtgtcccacgaggaccctgaggtgaagttcaactggtacgtggacggc |
| | | gtggaggtgcacaacgccaagaccaagcccagggaggaacagtacaac |
| | | tccacctaccgggtcgtgtccgtgctgaccgtcctgcaccaggactggctg |
| | | aacggcaaggagtacaagtgcaaggtgtccaacaaggccctgcccgccc |
| | | ccatcgagaagaccatctccaaggccaagggccagcctcgggagcccca |
| | | ggtgtacacactgcccccttccagggacgagctgaccaagaaccaggtgt |
| | | ccctgacctgtctggtgaagggcttctacccctccgacatcgccgtggagt |
| | | gggagtccaacggccagcccgagaacaattacaagaccacacctcccgt |
| | | cctggactccgacggctccttctttctgtactccaagctgaccgtggacaagt |
| | | ccaggtggcagcaaggcaacgtgttctcctgctccgtgatgcacgaggcc |
| | | ctgcacaaccactacacccagaagtccctgagcctgtcccccggc |
| 32 | 320-587 Light Chain | cagagcgtgctgacacagcctccatccgtgtctggcgcccctggccagag |
| | Variable Region | agtgaccatcagctgcaccagcagcagcagcgacatcggagccggcctg |
| | | ggcgtgcactggtatcagcagctgcctggcaccgcccccaagctgctgat |
| | | cgagggctactacaaccggcccagcggcgtgcccgaccggtttagcggc |
| | | agcaagagcggcaccagcgccagcctgacaatcaccggcctgctgcccg |
| | | aggacgagggcgactactactgccagagctgggacggcaccctgagcg |
| | | ccctgttcggcggaggcaccaagctgaccgtcctaggt |
| 33 | 320-587 Light Chain | cagagcgtgctgacacagcctccatccgtgtctggcgcccctggccagag |
| | | agtgaccatcagctgcaccagcagcagcagcgacatcggagccggcctg |
| | | ggcgtgcactggtatcagcagctgcctggcaccgcccccaagctgctgat |
| | | cgagggctactacaaccggcccagcggcgtgcccgaccggtttagcggc |
| | | agcaagagcggcaccagcgccagcctgacaatcaccggcctgctgcccg |
| | | aggacgagggcgactactactgccagagctgggacggcaccctgagcg |
| | | ccctgttcggcggaggcaccaagctgaccgtcctaggtcagcccaaggcc |
| | | gctcccagcgtgaccctgttccccccaagcagcgaggaactgcaggcca |
| | | acaaggccaccctggtgtgcctgatcagcgacttctaccctggggccgtga |
| | | ccgtggcctggaaggccgatagcagccctgtgaaggccggcgtggaaac |
| | | caccaccccctccaagcagagcaacaacaaatacgccgccagcagctac |
| | | ctgtccctgacccccgagcagtggaagtcccaccggtcctacagctgcca |
| | | ggtgacacacgagggcagcaccgtggaaaagaccgtggcccccaccga |
| | | gtgcagc |
| 34 | human TL1A | LKGQEFAPSHQQVYAPLRADGDKPRAHLTVVR |
| | | QTPTQHFKNQFPALHWEHELGLAFTKNRMNYT |
| | | NKFLLIPESGDYFIYSQVTFRGMTSECSEIRQAG |
| | | RPNKPDSITVVITKVTDSYPEPTQLLMGTKSVCE |
| | | VGSNWFQPIYLGAMFSLQEGDKLMVNVSDISLV |
| | | DYTKEDKTFFGAFLL |
| 35 | human TL1A | LKGQEFAPSHQQVYAPLRADGDKPRAHLTVVR |
| | | QTPTQHFKNQFPALHWEHELGLAFTKNRMNYT |
| | | NKFLLIPESGDYFIYSQVTFAGMTSECSEIRQAG |
| | | RPNKPDSITVVITKVTDSYPEPTQLLMGTKSVCE |
| | | VGSNWFQPIYLGAMFSLQEGDKLMVNVSDISLV |
| | | DYTKEDKTFFGAFLL |
| 36 | human TL1A | DYKDDDDKGSHHHHHHHHGSGSLVPRGSGSLK |
| | | GQEFAPSHQQVYAPLRADGDKPRAHLTVVRQT |
| | | PTQHFKNQFPALHWEHELGLAFTKNRMNYTNK |
| | | FLLIPESGDYFIYSQVTFRGMTSECSEIRQAGRPN |
| | | KPDSITVVITKVTDSYPEPTQLLMGTKSVCEVGS |
| | | NWFQPIYLGAMFSLQEGDKLMVNVSDISLVDY |
| | | TKEDKTFFGAFLL |
| 37 | cynomolgus monkey | LKGQEFAPSHQQVYAPLRADGDKPRAHLTVVR |
| | TL1A | QTPTQHLKNQFPALHWEHELGLAFTKNRMNYT |
| | | NKFLLIPESGDYFVYSQVTFRGMTSECSEIRQAG |
| | | RPNKPDSITVVITKVTDSYPEPTQLLMGTKSVCE |
| | | VGSNWFQPIYLGAMFSLQEGDKLMVNVSDISLV |
| | | DYTKEDKTFFGAFLL |
| 38 | mouse TL1A | LRAITEERSEPSPQQVYSPPRGKPRAHLTIKKQTP |
| | | APHLKNQLSALHWEHDLGMAFTKNGMKYINK |
| | | SLVIPESGDYFIYSQITFRGTTSVCGDISRGRRPN |
| | | KPDSITMVITKVADSYPEPARLLTGSKSVCEISN |
| | | NWFQSLYLGATFSLEEGDRLMVNVSDISLVDYT |
| | | KEDKTFFGAFLL |
| 39 | rat TL1A | FPTVTEERSAPSAQPVYTPSRDKPKAHLTIMRQT |
| | | PVPHLKNELAALHWENNLGMAFTKNRMNYTN |
| | | KFLVIPESGDYFIYSQITFRGTTSECGDISRVRRP |
| | | KKPDSITVVITKVADSYPEPAHLLTGTKSVCEISS |
| | | NWFQPIYLGAMFSLEEGDRLMVNVSDISLVDYT |
| | | KEDKTFFGAFLI |
| 40 | guinea pig TL1A | INEQRFGPSYQRVYTPLRDDRDKPRAHLTVVRQ |
| | | TPTQHLKNQFPALHWEHELGLAFTKNRMNYTN |
| | | KFLVIPETGDYFVYSQITFRGTTSECGISPGRQQN |
| | | KPDSIFVVITKVTDSYPEPSQLLTGTKSVCEISSN |
| | | WFQPLYLGAMFSLQEGDKLMVNVSDISLVDYT |
| | | KEDKTFFGAFLL |
| 41 | cat TL1A | PKGREFGPSHQRAYTSPGAGGDKPRAHLTVVR |
| | | QTPTQPLKNQFPALHWEHELGLAFIKNRMNYT |
| | | NKFLVIPESGDYFVYSQVTFRGTTSECGEIRQGS |
| | | RLNKPDSIIVVITKVTDSYPEPTQLLMGTKSVCE |
| | | VGSNWFQPIYLGAMFSLQEGDKLMVNVSDISLV |
| | | DYTKEDKTFFGAFLL |
| 42 | pig TLIA | PKGQELGPSHQRVYAPPGAGRDKPRAHLTVVR |
| | | QTSTEPLKNQFPALHWEHELGLAFTKNRMNYT |
| | | NKFLVIPESGDYFIYSQVTFRGTTSECGEISQERR |
| | | LNKPDSIIVVITKVTDSYPEPTQLLMGTKSVCEIG |
| | | SNWFQPIYLGAMFSLHEGDKLMVNVSDISLVDY |
| | | TKEDKTFFGAFLL |
| 43 | rabbit TL1A | LKGREFGPSQQRAYMPLRADGNKPRAHLTAVK |
| | | QTPTQPLRNHFPALHWEHELGLAFTKNRMNYT |
| | | NKFLVIPESGDYFVYSQVTFRGTTSECGVINQRR |
| | | RQTKPDSIVVVITKVTDNYPEPAQLLTGTKSVCE |
| | | MGNWFQPIYLGAMFSLEEGDKLMVNVSDVSLV |
| | | DYTKEDKTFFGAFLL |
| 44 | dog TL1A | PKGQEFGHSHQRAYASPRAGGDKPRAHLTVVR |
| | | QSPTQPLESLFPALHWEHELGLAFTKNRMNYTN |
| | | KFLVIPESGDYFVYSQVTFRGTTSECGEARQGSR |
| | | LNKPDSIIVVITKVTDSYPEPTQLLMGTKSVCEV |
| | | GSNWFQPIYLGAMFSLQEGDKLMVNVSDISLVD |
| | | YTKEDKTFFGAFLL |

The invention claimed is:

1. A method of treating moderate to severe ulcerative colitis or moderate to severe Crohn's disease in a subject in need thereof, comprising administering to the subject an antibody or antigen-binding fragment thereof that specifically binds to TNF-like ligand 1A (TL1A);
wherein the antibody or antigen-binding fragment comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6.

2. The method of claim 1, wherein the subject was treated previously with one or more agents selected from the group consisting of a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, a sphingosine-1-phosphate (S1P) receptor modulator, and 5-aminosalicylic acid (5-ASA).

3. The method of claim 2, wherein the subject had an inadequate response to, loss of response to, or intolerance of one or more of the agents.

4. The method of claim 3, wherein the subject had an inadequate response to, loss of response to, or intolerance of no more than two classes of biologics.

5. A method of treating ulcerative colitis or Crohn's disease in a subject in need thereof, the method comprising
administering to a subject in need thereof a composition comprising an antibody or antigen-binding fragment thereof that specifically binds to TNF-like ligand 1A (TL1A);
wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; and
wherein the subject previously had an inadequate response to, loss of response to, or intolerance to at least one agent selected from the following group: a corticosteroid, an immunosuppressant, a tumor necrosis factor-alpha (TNF-α) antagonist, an anti-integrin antibody, an anti-interleukin (IL)-12/23 antibody, a Janus kinase (JAK) inhibitor, and a sphingosine-1-phosphate (SIP) receptor modulator.

6. (canceled)

7. A method of treating moderate to severe ulcerative colitis or moderate to severe Crohn's disease in a subject in need thereof, the method comprising:
administering to the subject a composition comprising an antibody or antigen-binding fragment thereof that specifically binds to TNF-like ligand 1A (TL1A);
wherein the antibody or antigen-binding fragment thereof comprises: a heavy chain variable region CDR1 comprising the amino acid sequence of SEQ ID NO: 1, a heavy chain variable region CDR2 comprising the amino acid sequence of SEQ ID NO: 2, a heavy chain variable region CDR3 comprising the amino acid sequence of SEQ ID NO: 3, a light chain variable region CDR1 comprising the amino acid sequence of SEQ ID NO: 4, a light chain variable region CDR2 comprising the amino acid sequence of SEQ ID NO: 5, and a light chain variable region CDR3 comprising the amino acid sequence of SEQ ID NO: 6; and
wherein the antibody or antigen-binding fragment thereof is administered at a dose of about 300 mg to about 2250 mg.

8. (canceled)

9. (canceled)

10. (canceled)

11. The method of claim 1, wherein the antibody or antigen-binding fragment comprises a heavy chain variable region comprising the amino acid sequence of SEQ ID NO: 7 and a light chain variable region comprising the amino acid sequence of SEQ ID NO: 8.

12. The method of claim 1, wherein the antibody or antigen-binding fragment comprises an IgG1 constant region.

13. The method of claim 1, wherein the antibody or antigen-binding fragment comprises a heavy chain comprising the amino acid sequence of SEQ ID NO: 9 and a light chain comprising the amino acid sequence of SEQ ID NO: 10.

14. The method of claim 1, wherein the subject is treated concurrently with an oral corticosteroid, an oral 5-ASA, sulfasalazine, azathioprine (AZA), 6-mercaptopurine (6-MP), and/or methotrexate.

15. The method of claim 1, wherein the antibody or antigen-binding fragment thereof is administered at dose of about 300 mg, about 400 mg, about 450 mg, about 600 mg, about 750 mg, about 900 mg, or about 2250 mg and/or is administered about once every two weeks or about once every four weeks.

16. (canceled)

17. (canceled)

18. (canceled)

19. (canceled)

20. The method of claim 1, wherein the antibody or antigen-binding fragment thereof is administered at a dose of about 900 mg.

21. (canceled)

22. (canceled)

23. (canceled)

24. The method of claim 1, wherein the administration comprises administering:
(a) one or more induction doses of the antibody or antigen-binding fragment thereof, and wherein no loading dose is administered; or
(b) a loading dose of the antibody or antigen-binding fragment thereof, followed by administration of one or more induction doses of the antibody or antigen-binding fragment thereof.

25. (canceled)

26. (canceled)

27. The method of claim 24, wherein the one or more induction doses comprises about 450 mg or about 900 mg of the antibody or antigen-binding fragment thereof, about six induction doses are administered, the one or more induction doses occurs over about 14 weeks, and/or the loading dose is administered about 2 weeks before the one or more induction doses are administered.

28. (canceled)

29. (canceled)

30. The method of claim 1, wherein the administration occurs over about 16 weeks.

31. The method of claim 1, wherein the administration comprises administering one or more maintenance doses of the antibody or antigen-binding fragment thereof, and wherein no loading dose and/or induction dose is administered, optionally wherein:
(a) the maintenance doses are administered about every 2 weeks or about every 4 weeks,
(b) the one or more maintenance doses comprises about 450 mg or about 900 mg of the antibody or antigen-binding fragment thereof;
(c) about ten maintenance doses are administered; and/or
(d) the administration of the one or more maintenance doses occurs over about 24 weeks or about 40 weeks.

32. The method of claim 1, wherein the administration further comprises administering one or more maintenance doses of the antibody or antigen-binding fragment thereof, optionally wherein:
(a) the maintenance doses are administered about every 2 weeks or about every 4 weeks,
(b) the one or more maintenance doses comprises about 450 mg or about 900 mg of the antibody or antigen-binding fragment thereof;
(c) about ten maintenance doses are administered; and/or
(d) the administration of the one or more maintenance doses occurs over about 24 weeks or about 40 weeks.

33. (canceled)

34. (canceled)

35. (canceled)

36. (canceled)

37. (canceled)

38. (canceled)

39. The method of claim 1, wherein the administration comprises administering:
a) (i) a loading dose of about 1500 mg to about 2500 mg, (ii) one or more induction doses of about 300 mg to about 1800 mg, and (iii) one or more maintenance doses of about 300 mg to about 2250 mg of the antibody or antigen-binding fragment thereof, optionally wherein the induction doses are administered about every two weeks, the maintenance doses are administered about every two weeks or about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
b) (i) a loading dose of about 2250 mg, (ii) one or more induction doses of about 450 mg, and (iii) one or more maintenance doses of about 450 mg of the antibody or antigen-binding fragment thereof, optionally wherein the induction doses are administered about every two weeks, the maintenance doses are administered about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
c) (i) a loading dose of about 2250 mg, (ii) one or more induction doses of about 900 mg, and (iii) one or more maintenance doses of about 450 mg of the antibody or antigen-binding fragment thereof, optionally wherein the induction doses are administered about every two weeks, the maintenance doses are administered about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
d) (i) a loading dose of about 2250 mg, (ii) one or more induction doses of about 450 mg, and (iii) one or more maintenance doses of about 900 mg of the antibody or antigen-binding fragment thereof, optionally wherein the induction doses are administered about every two weeks, the maintenance doses are administered about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
e) (i) a loading dose of about 2250 mg, (ii) one or more induction doses of about 900 mg, and (iii) one or more maintenance doses of about 900 mg of the antibody or antigen-binding fragment thereof, optionally wherein the induction doses are administered about every two weeks, the maintenance doses are administered about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
f) (i) a loading dose of about 1750 mg, (ii) one or more induction doses of about 900 mg, and (iii) one or more maintenance doses of about 900 mg of the antibody or antigen-binding fragment thereof, optionally wherein the induction doses are administered about every two weeks, the maintenance doses are administered about every two weeks or about every four weeks, about 6 induction doses are administered, about ten maintenance doses are administered, the one or more induction doses occurs over about 14 weeks, the loading dose is administered about 2 weeks before the one or more induction doses are administered, and/or the administration of the one or more maintenance doses occurs over about 24 weeks or about 40 weeks;
g) (i) a loading dose of about 1500 mg to about 2500 mg and (ii) one or more maintenance doses of about 300 mg to about 2250 mg of the antibody or antigen-binding fragment thereof, optionally wherein the maintenance doses are administered about every two weeks or about every four weeks, at least ten maintenance doses are administered, and/or the administration of the one or more maintenance doses occurs over about 24 weeks or about 40 weeks;
h) (i) a loading dose of about 2250 mg and (ii) one or more maintenance doses of about 450 mg of the antibody or antigen-binding fragment thereof, optionally wherein the maintenance doses are administered about every four weeks, at least ten maintenance doses are administered, and/or the administration of the one or more maintenance doses occurs over about 24 weeks or about 40 weeks;
i) (i) a loading dose of about 2250 mg and (ii) one or more maintenance doses of about 900 mg of the antibody or antigen-binding fragment thereof, optionally wherein the maintenance doses are administered about every four weeks, at least ten maintenance doses are administered, and/or the administration of the one or more maintenance doses occurs over about 24 weeks or about 40 weeks; or
j) (i) a loading dose of about 1750 mg and (ii) one or more maintenance doses of about 900 mg of the antibody or antigen-binding fragment thereof, optionally wherein the maintenance doses are administered about every two weeks or about every four weeks, at least ten maintenance doses are administered, and/or the administration of the one or more maintenance doses occurs over about 24 weeks or about 40 weeks.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. The method of claim 1, wherein the antibody or antigen-binding fragment thereof is:
a) formulated at a concentration of about 200 mg/mL;
b) formulated in a volume of 3 mL or less;
c) administered to the subject subcutaneously or intravenously; and/or
d) administered to the subject via a syringe, optionally wherein the syringe is a pre-filled syringe, further optionally wherein the pre-filled syringe is in a volume of 2 mL or less.

53. (canceled)

54. (canceled)

55. (canceled)

56. (canceled)

57. (canceled)

58. The method of claim 1, wherein the subject has:
(a) moderate to severe ulcerative colitis, optionally wherein the administration results in clinical remission, further optionally wherein the clinical remission occurs within 14 weeks or within 38 weeks of the start of the administration; and/or
(b) moderate to severe Crohn's disease, optionally wherein the administration results in an endoscopic response, further optionally wherein the endoscopic response occurs within 14 weeks or within 38 weeks of the start of the administration.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)


SEQUENCE LISTING (44 sequences)

SEQ ID NO: 1 - protein, 10 aa, synthetic construct
  REGION 1..10: note = 320-587 Heavy Chain CDR1
GYTFTSYDIN

SEQ ID NO: 2 - protein, 10 aa, synthetic construct
  REGION 1..10: note = 320-587 Heavy Chain CDR2
WLNPNSGYTG

SEQ ID NO: 3 - protein, 10 aa, synthetic construct
  REGION 1..10: note = 320-587 Heavy Chain CDR3
EVPETAAFEY

SEQ ID NO: 4 - protein, 14 aa, synthetic construct
  REGION 1..14: note = 320-587 Light Chain CDR1
TSSSSDIGAGLGVH

SEQ ID NO: 5 - protein, 7 aa, synthetic construct
  REGION 1..7: note = 320-587 Light Chain CDR2
GYYNRPS

SEQ ID NO: 6 - protein, 10 aa, synthetic construct
  REGION 1..10: note = 320-587 Light Chain CDR3
QSWDGTLSAL

SEQ ID NO: 7 - protein, 119 aa, synthetic construct
  REGION 1..119: note = 320-587 Heavy Chain Variable Region
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 8 - protein, 111 aa, synthetic construct
  REGION 1..111: note = 320-587 Light Chain Variable Region
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 9 - protein, 448 aa, synthetic construct
  REGION 1..448: note = 320-587 Heavy Chain
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 216 aa, synthetic construct
  REGION 1..216: note = 320-587 Light Chain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 119 aa, synthetic construct
  REGION 1..119: note = 320-179 Heavy Chain Variable Region
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 12 - protein, 111 aa, synthetic construct
  REGION 1..111: note = 320-179 Light Chain Variable Region
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 13 - protein, 330 aa, synthetic construct [sequence omitted]

SEQ ID NO: 14 - protein, 329 aa, synthetic construct
  REGION 1..329: note = IgG1 dK HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 330 aa, synthetic construct
  REGION 1..330: note = IgG1 252Y/254T/256E HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 329 aa, synthetic construct
  REGION 1..329: note = IgG1 dK + 252Y/254T/256E HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 330 aa, synthetic construct
  REGION 1..330: note = IgG1 L234A, L235A, G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 329 aa, synthetic construct
  REGION 1..329: note = IgG1 dK + L234A, L235A, G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 330 aa, synthetic construct
  REGION 1..330: note = IgG1 L235A/G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 329 aa, synthetic construct
  REGION 1..329: note = IgG1 dK + L235A/G237A HC constant region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 326 aa, synthetic construct
  REGION 1..326: note = IgG2 HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 325 aa, synthetic construct
  REGION 1..325: note = IgG2 dK HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 23 - protein, 326 aa, synthetic construct
  REGION 1..326: note = IgG2 A330S/P331S HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 325 aa, synthetic construct
  REGION 1..325: note = IgG2 dK + A330S/P331S HC constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 25 - protein, 327 aa, synthetic construct
  REGION 1..327: note = IgG4 HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 326 aa, synthetic construct
  REGION 1..326: note = IgG4 dK + S228P HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 327 aa, synthetic construct
  REGION 1..327: note = IgG4 S228P + 252Y/254T/256E HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 326 aa, synthetic construct
  REGION 1..326: note = IgG4 dK + S228P + 252Y/254T/256E HC constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 105 aa, synthetic construct
  REGION 1..105: note = Lambda LC constant region
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - DNA, 357 bp, synthetic construct
  source 1..357: mol_type = other DNA
  misc_feature 1..357: note = 320-587 Heavy Chain Variable Region
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagc

SEQ ID NO: 31 - DNA, 1344 bp, synthetic construct
  source 1..1344: mol_type = other DNA
  misc_feature 1..1344: note = 320-587 Heavy Chain
caggtgcagctggtgcagtccggcgccgaggtgaagaaacccggcgcctccgtgaaggtgtcctgcaaggccagcggctacaccttcacctcctacgacatcaactgggtgaggcaggcccccggccagggcctggagtggatgggctggctgaaccccaactccggctacaccggctacgcccagaagttccagggcagggtgaccatgaccgccgacaggtccacctccaccgcctacatggagctgtccagcctgaggtccgaggacaccgccgtgtactattgcgccagggaggtgcccgagaccgctgccttcgagtactggggccagggcaccctggtgaccgtgtccagcgcctccaccaagggccccagcgtgttccccctggcccccagctccaagtccaccagcggcggaaccgccgctctgggctgcctggtgaaggactacttccccgagcccgtgaccgtgagctggaactccggcgccctgacctccggcgtgcacaccttccccgccgtgctgcagtccagcggcctgtactccctgagctccgtggtcaccgtgccctcctctagcctgggcacccagacctacatctgcaacgtgaaccacaagccctccaacaccaaggtggacaaaaaggtggagcccaagtcctgcgacaagactcacacctgtcctccctgccccgcccccgagctgctcggcggaccctccgtgttcctgttcccacccaagcccaaggacaccctgatgatctccaggacccccgaggtgacctgcgtggtcgtggacgtgtcccacgaggaccctgaggtgaagttcaactggtacgtggacggcgtggaggtgcacaacgccaagaccaagcccagggaggaacagtacaactccacctaccgggtcgtgtccgtgctgaccgtcctgcaccaggactggctgaacggcaaggagtacaagtgcaaggtgtccaacaaggccctgcccgcccccatcgagaagaccatctccaaggccaagggccagcctcgggagccccaggtgtacacactgcccccttccagggacgagctgaccaagaaccaggtgtccctgacctgtctggtgaagggcttctacccctccgacatcgccgtggagtgggagtccaacggccagcccgagaacaattacaagaccacacctcccgtcctggactccgacggctccttctttctgtactccaagctgaccgtggacaagtccaggtggcagcaaggcaacgtgttctcctgctccgtgatgcacgaggccctgcacaaccactacacccagaagtccctgagcctgtcccccggc

SEQ ID NO: 32 - DNA, 333 bp, synthetic construct
  source 1..333: mol_type = other DNA
  misc_feature 1..333: note = 320-587 Light Chain Variable Region
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggt

SEQ ID NO: 33 - DNA, 648 bp, synthetic construct
  source 1..648: mol_type = other DNA
  misc_feature 1..648: note = 320-587 Light Chain
cagagcgtgctgacacagcctccatccgtgtctggcgcccctggccagagagtgaccatcagctgcaccagcagcagcagcgacatcggagccggcctgggcgtgcactggtatcagcagctgcctggcaccgcccccaagctgctgatcgagggctactacaaccggcccagcggcgtgcccgaccggtttagcggcagcaagagcggcaccagcgccagcctgacaatcaccggcctgctgcccgaggacgagggcgactactactgccagagctgggacggcaccctgagcgccctgttcggcggaggcaccaagctgaccgtcctaggtcagcccaaggccgctcccagcgtgaccctgttccccccaagcagcgaggaactgcaggccaacaaggccaccctggtgtgcctgatcagcgacttctaccctggggccgtgaccgtggcctggaaggccgatagcagccctgtgaaggccggcgtggaaaccaccaccccctccaagcagagcaacaacaaatacgccgccagcagctacctgtccctgacccccgagcagtggaagtcccaccggtcctacagctgccaggtgacacacgagggcagcaccgtggaaaagaccgtggcccccaccgagtgcagc

SEQ ID NO: 34 - protein, 180 aa, synthetic construct
  REGION 1..180: note = human TL1A
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 35 - protein, 180 aa, synthetic construct
  REGION 1..180: note = human TL1A
LKGQEFAPSHQQVYAPLRADGDKPRAHLTVVRQTPTQHFKNQFPALHWEHELGLAFTKNRMNYTNKFLLIPESGDYFIYSQVTFAGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 36 - protein, 210 aa, synthetic construct
  REGION 1..210: note = human TL1A
DYKDDDDKGSHHHHHHHHGSGSLVPRGSGSLKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 37 - protein, 180 aa, synthetic construct
  REGION 1..180: note = cynomolgus monkey TL1A
LKGQEFAPSHQQVYAPLRADGDKPRAHLTVVRQTPTQHLKNQFPALHWEHELGLAFTKNRMNYTNKFLLIPESGDYFVYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 38 - protein, 180 aa, synthetic construct
  REGION 1..180: note = mouse TL1A
LRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITMVITKVADSYPEPARLLTGSKSVCEISNNWFQSLYLGATFSLEEGDRLMVNVSDISLVDYTKEDKTFFGAFLL

SEQ ID NO: 39 - protein, 180 aa, synthetic construct
  REGION 1..180: note = rat TL1A
FPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 40 - protein, 179 aa, synthetic construct
  REGION 1..179: note = guinea pig TL1A
INEQRFGPSYQRVYTPLRDDRDKPRAHLTVVRQTPTQHLKNQFPALHWEHELGLAFTKNRMNYTNKFLVIPETGDYFVYSQITFRGTTSECGISPGRQQNKPDSIFVVITKVTDSYPEPSQLLTGTKSVCEISSNWFQPLYLGAMFSLQEGDKLMVNVSDISLVDYTKEDKTFFGAFLL

SEQ ID NO: 41 - protein, 180 aa, synthetic construct
  REGION 1..180: note = cat TL1A
PKGREFGPSHQRAYTSPGAGGDKPRAHLTVVRQTPTQPLKNQFPALHWEHELGLAFIKNRMNYTNKFLVIPESGDYFVYSQVTFRGTTSECGEIRQGSRLNKPDSIIVVITKVTDSYPEPTQLLMGTKSVCEVGSNWFQPIYLGAMFSLQEGDKLMVNVSDISLVDYTKEDKTFFGAFLL

SEQ ID NO: 42 - protein, 180 aa, synthetic construct
  REGION 1..180: note = pig TL1A
PKGQELGPSHQRVYAPPGAGRDKPRAHLTVVRQTSTEPLKNQFPALHWEHELGLAFTKNRMNYTNKFLVIPESGDYFIYSQVTFRGTTSECGEISQERRLNKPDSIIVVITKVTDSYPEPTQLLMGTKSVCEIGSNWFQPIYLGAMFSLHEGDKLMVNVSDISLVDYTKEDKTFFGAFLL

SEQ ID NO: 43 - protein, 179 aa, synthetic construct
  REGION 1..179: note = rabbit TL1A
LKGREFGPSQQRAYMPLRADGNKPRAHLTAVKQTPTQPLRNHFPALHWEHELGLAFTKNRMNYTNKFLVIPESGDYFVYSQVTFRGTTSECGVINQRRRQTKPDSIVVVITKVTDNYPEPAQLLTGTKSVCEMGNWFQPIYLGAMFSLEEGDKLMVNVSDVSLVDYTKEDKTFFGAFLL

SEQ ID NO: 44 - protein, 180 aa, synthetic construct
  REGION 1..180: note = dog TL1A
PKGQEFGHSHQRAYASPRAGGDKPRAHLTVVRQSPTQPLESLFPALHWEHELGLAFTKNRMNYTNKFLVIPESGDYFVYSQVTFRGTTSECGEARQGSRLNKPDSIIVVITKVTDSYPEPTQLLMGTKSVCEVGSNWFQPIYLGAMFSLQEGDKLMVNVSDISLVDYTKEDKTFFGAFLL